(19) United States
(12) Patent Application Publication
(10) Pub. No.: US 2018/0106797 A1
(43) Pub. Date: Apr. 19, 2018

(54) MULTIPLEXED VOLUMETRIC BAR CHART CHIP FOR POINT OF CARE BIOMARKER AND/OR ANALYTE QUANTITATION, INCLUDING COMPETITIVE CONTROL

(71) Applicant: The Methodist Hospital, Houston, TX (US)

(72) Inventors: Lidong Qin, Houston, TX (US); Ying Li, Houston, TX (US); Ping Wang, Houston, TX (US); Xifeng Wu, Pearland, TX (US)

(21) Appl. No.: 15/559,968

(22) PCT Filed: Mar. 21, 2016
(86) PCT No.: PCT/US2016/023416

§ 371 Date: Sep. 20, 2017

Related U.S. Application Data
(63) Continuation-in-part of application No. 14/435,997, filed on Apr. 15, 2015.
Application No. 14/435,997 is a continuation of application No. 13/834,614, filed on Mar. 15, 2013, now Pat. No. 9,097,710.
Application No. PCT/US2016/023416 is a continuation-in-part of application No. 14/817,258, filed on Aug. 4, 2015.
Application No. 14/817,258 is a continuation of application No. 13/834,614, filed on Mar. 15, 2013, now Pat. No. 9,097,710.
(60) Provisional application No. 62/136,218, filed on Mar. 20, 2015.
(60) Provisional application No. 61/714,676, filed on Oct. 16, 2012.
(60) Provisional application No. 61/714,676, filed on Oct. 16, 2012.

(51) Int. Cl. G01N 33/543; C12Q 1/6823; B01L 3/00

(52) U.S. Cl. CPC G01N 33/54386; B01L 2400/065; B01L 2400/0406; B01L 2300/0887; B01L 2300/0867; B01L 2300/0816; B01L 2200/0605; B01L 3/502738; G01N 33/54366; G01N 33/54306; C12Q 1/6823

(57) ABSTRACT

Apparatus for determining the quantity of a target analyte present in a sample, the apparatus comprising: a first plate comprising a plurality of recesses arranged to form a plurality of rows extending parallel to one another, and a plurality of channels extending perpendicularly to the plurality of rows of the first plate; and a second plate comprising a plurality of recesses arranged to form a plurality of rows extending parallel to one another, and a plurality of channels extending perpendicularly to the plurality of rows of the second plate; wherein the first plate and the second plate are assembled together so that the first plate is positioned against the second plate and the recesses of the first plate communicate with the recesses of the second plate so as to form a plurality of sample rows, a plurality of control rows, and an ink row disposed between the plurality of sample rows and the plurality of control rows.

### REFERENCE TO PENDING PRIOR PATENT APPLICATIONS

This patent application:

(i) is a continuation-in-part of pending prior U.S. patent application Ser. No. 14/817,258, filed Aug. 4, 2015 by The Methodist Hospital Research Institute and Lidong Qin et al. for MULTIPLEXED VOLUMETRIC BAR CHART CHIP FOR POINT OF CARE BIOMARKER AND ANALYTE QUANTITATION (Attorney's Docket No. METHODIST-4 CON), which patent application is a continuation of prior U.S. patent application Ser. No. 13/834,614, filed Mar. 15, 2013 by The Methodist Hospital Research Institute and Lidong Qin et al. for MULTIPLEXED VOLUMETRIC BAR CHART CHIP FOR POINT OF CARE BIOMARKER AND ANALYTE QUANTITATION (Attorney's Docket No. METHODIST-4), which in turn claims benefit of prior U.S. Provisional Patent Application Ser. No. 61/714,676, filed Oct. 16, 2012 by The Methodist Hospital Research Institute and Lidong Qin et al. for MULTIPLEXED VOLUMETRIC BAR CHART CHIP FOR POINT OF CARE BIOMARKER QUANTITATION (Attorney's Docket No. METHODIST-4 PROV);

(ii) is a continuation-in-part of pending prior U.S. patent application Ser. No. 14/435,997, filed Apr. 15, 2015 by The Methodist Hospital and Lidong Qin et al. for MULTIPLEXED VOLUMETRIC BAR CHART CHIP FOR POINT OF CARE BIOMARKER AND/OR ANALYTE QUANTITATION (Attorney's Docket No. METHODIST-4 PCT US), which patent application is a U.S. national stage entry of International (PCT) Patent Application No. PCT/US13/65270, filed Oct. 16, 2013 by The Methodist Hospital for MULTIPLEXED VOLUMETRIC BAR CHART CHIP FOR POINT OF CARE BIOMARKER AND/OR ANALYTE QUANTITATION (Attorney's Docket No. METHODIST-4 PCT), which patent application claims benefit of: (a) prior U.S. patent application Ser. No. 13/834,614, filed Mar. 15, 2013 by The Methodist Hospital Research Institute and Lidong Quin et al. for MULTIPLEXED VOLUMETRIC BAR CHART CHIP FOR POINT OF CARE BIOMARKER AND ANALYTE QUANTITATION (Attorney's Docket No. METHODIST-4), which in turn claims benefit of prior U.S. Provisional Patent Application Ser. No. 61/714,676, filed Oct. 16, 2012 by The Methodist Hospital Research Institute and Lidong Qin et al. for MULTIPLEXED VOLUMETRIC BAR CHART CHIP FOR POINT OF CARE BIOMARKER QUANTITATION (Attorney's Docket No. METHODIST-4 PROV), and (b) prior U.S. Provisional Patent Application Ser. No. 61/714,676, filed Oct. 16, 2012 by The Methodist Hospital Research Institute and Lidong Qin et al. for MULTIPLEXED VOLUMETRIC BAR CHART CHIP FOR POINT OF CARE BIOMARKER QUANTITATION (Attorney's Docket No. METHODIST-4 PROV); and

(iii) claims benefit of pending prior U.S. Provisional Patent Application Ser. No. 62/136,218, filed Mar. 20, 2015 by The Methodist Hospital and Lidong Qin et al. for MULTIPLEXED VOLUMETRIC BAR CHART CHIP FOR POINT OF CARE BIOMARKER AND/OR ANALYTE QUANTITATION (Attorney's Docket No. METHODIST-1618 PROV).

The six (6) above-identified patent applications are hereby incorporated herein by reference.

### FIELD OF THE INVENTION

This invention relates generally to methods and apparatus for determining the quantity of a protein and/or other biomarkers and/or analytes present in a sample, and more particularly to methods and apparatus for point of care determination of the quantity of a protein (and, preferably, the quantity of multiple biomarkers) present in a sample.

### BACKGROUND OF THE INVENTION

Molecular quantity analysis is widely used in research, diagnosis, quality control and other types of measurements. It is well known that the diagnosis and treatment of certain medical conditions can be facilitated by identifying the presence and quantity of a selected biomarker in a sample taken from a patient. Furthermore, research has shown that, in many situations, multi-biomarker measurements can provide a more accurate diagnostic result. More particularly, biomarker research has identified many helpful proteomics and genomic panels for disease diagnosis and prognosis, including cancer, infection, cardiovascular disease, diabetes, Alzheimer's disease and others. For example, a four-biomarker panel has been developed for detecting early stage ovarian cancer, and an 18-protein biomarker panel has been developed for the diagnosis of early Alzheimer's disease.

Current methods for protein-based biomarker assays typically utilize an enzyme-linked immunosorbent assay (ELISA) approach, where the target protein binds to a specific recognition molecule, and then colorimetric, fluorescent, electrochemical or magnetic signals are introduced to transduce the binding event into a readout signal. However, inasmuch as advanced instrumentation is typically required for quantitative detection of the target protein, these methods are not ideal for point of care applications, due to the size and high cost of the instrumentation and/or the complicated operation of the instrumentation. See, for example, FIG. 1, which shows the typical approach for a protein-based biomarker assay, where a blood sample is drawn from a patient and then processed by a relatively large, complex instrument.

Thus there is a need for a new method and apparatus for point of care determination of the quantity of a protein (and, preferably, the quantity of multiple proteins) present in a sample.

### SUMMARY OF THE INVENTION

These and other objects are addressed by the provision and use of a novel method and apparatus for point of care determination of the quantity of a protein (and, preferably, the quantity of multiple proteins) present in a sample.

In one form of the present invention, there is provided apparatus for determining the quantity of a target protein and/or other types of biomarkers or analytes present in a sample, the apparatus comprising:

a top plate comprising a plurality of recesses arranged to form a plurality of rows extending parallel to one another; and

a bottom plate comprising a plurality of recesses arranged to form a plurality of rows extending parallel to one another, and a plurality of channels extending perpendicularly to the plurality of rows of the bottom plate;

wherein the top plate and the bottom plate are assembled together so that the top plate is on top of the bottom plate and the recesses of the top plate communicate with the recesses of the bottom plate so as to form a plurality of rows; and

wherein at least one of the top plate and the bottom plate is configured to slide relative to the other of the top plate and the bottom plate in order to form a plurality of columns, with each of the plurality of columns in communication with each of the plurality of channels.

In another form of the present invention, there is provided a method for determining the quantity of a target protein and/or other types of biomarkers or analytes present in a sample, the method comprising:

providing apparatus comprising:
a top plate comprising a plurality of recesses arranged to form a plurality of rows extending parallel to one another; and
a bottom plate comprising a plurality of recesses arranged to form a plurality of rows extending parallel to one another, and a plurality of channels extending perpendicularly to the plurality of rows of the bottom plate;
wherein the top plate and the bottom plate are assembled together so that the top plate is on top of the bottom plate and the recesses of the top plate communicate with the recesses of the bottom plate so as to form a plurality of rows; and
wherein at least one of the top plate and the bottom plate is configured to slide relative to the other of the top plate and the bottom plate in order to form a plurality of columns, with each of the plurality of columns in communication with each of the plurality of channels;

binding a protein-specific antibody in at least one recess forming one of the plurality of rows of the top plate;

positioning hydrogen peroxide in a recess adjacent to the row containing the protein-specific antibody;

positioning ink in a recess in a row adjacent to the plurality of channels;

positioning a sample in the at least one recess containing the protein-specific antibody;

positioning a catalase in the at least one recess containing the protein-specific antibody and the sample;

sliding one of the top plate and the bottom plate relative to the other of the top plate and the bottom plate so as to form the plurality of columns, with each column being in communication with one of the plurality of channels; and

determining the quantity of the target protein and/or other biomarker and/or other molecular analyte present in the sample by detecting the longitudinal position of the ink contained in the plurality of channels.

In another form of the present invention, there is provided a method for determining the quantity of a target analyte present in a sample, the method comprising:

providing apparatus comprising:
a top plate comprising a plurality of recesses arranged to form a plurality of rows extending parallel to one another; and
a bottom plate comprising a plurality of recesses arranged to form a plurality of rows extending parallel to one another, and a plurality of channels extending perpendicularly to the plurality of rows of the bottom plate;
wherein the top plate and the bottom plate are assembled together so that the top plate is on top of the bottom plate and the recesses of the top plate communicate with the recesses of the bottom plate so as to form a plurality of rows; and
wherein at least one of the top plate and the bottom plate is configured to slide relative to the other of the top plate and the bottom plate in order to form a plurality of columns, with each of the plurality of columns in communication with each of the plurality of channels;

binding a capture agent in at least one recess forming one of the plurality of rows of the top plate, introducing a sample into the at least one recess so that an analyte contained in the sample is bound to the capture agent, and binding a probe to the bound analyte; and positioning a reagent in a recess adjacent to the row containing the capture agent, bound analyte and bound probe; and positioning ink in a recess in a row adjacent to the plurality of channels;

sliding one of the top plate and the bottom plate relative to the other of the top plate and the bottom plate so as to form the plurality of columns, with each column being in communication with one of the plurality of channels; and

determining the quantity of the analyte present in the sample by detecting the longitudinal position of the ink contained in the plurality of channels.

In another form of the present invention, there is provided apparatus for determining the quantity of a target protein and/or other types of biomarkers or analytes present in a sample, the apparatus comprising:

a top plate comprising at least one recess; and

a bottom plate comprising at least one recess, and at least one serpentine channel communicating with the at least one recess of the bottom plate;

wherein at least one of the top plate and the bottom plate is configured to slide relative to the other of the top plate and the bottom plate in order to align the at least one recess of the top plate with the at least one recess of the bottom plate, so that there exists fluid communication between the at least one recess of the top plate, the at least one recess of the bottom plate, and the at least one serpentine channel communicating with the at least one recess of the bottom plate.

In another form of the present invention, there is provided apparatus for determining the quantity of a target analyte present in a sample, the apparatus comprising:

a first plate comprising a plurality of recesses arranged to form a plurality of rows extending parallel to one another, and a plurality of channels extending perpendicularly to the plurality of rows of the first plate; and

a second plate comprising a plurality of recesses arranged to form a plurality of rows extending parallel to one another, and a plurality of channels extending perpendicularly to the plurality of rows of the second plate;

wherein the first plate and the second plate are assembled together so that the first plate is positioned against the second plate and the recesses of the first plate communicate with the recesses of the second plate so as to form a plurality of sample rows, a plurality of control rows, and an ink row disposed between the plurality of sample rows and the plurality of control rows, with the plurality of channels of the first plate being disposed between the plurality of control rows and the ink row, and the plurality of channels of the second plate being disposed between the plurality of sample rows and the ink row; and

wherein at least one of the first plate and the second plate is configured to slide relative to the other of the first plate and the second plate in order to form a plurality of sample columns, a plurality of control columns and a plurality of ink columns, with each of the plurality of channels in the second plate being in communication with each of the plurality of sample columns and ink columns and with each of the plurality of channels in the first plate being in communication with each of the plurality of control columns and ink columns.

In another form of the present invention, there is provided a method for determining the quantity of a target analyte present in a sample, the method comprising:

providing apparatus comprising:
a first plate comprising a plurality of recesses arranged to form a plurality of rows extending parallel to one another, and a plurality of channels extending perpendicularly to the plurality of rows of the first plate; and
a second plate comprising a plurality of recesses arranged to form a plurality of rows extending parallel to one another, and a plurality of channels extending perpendicularly to the plurality of rows of the second plate;
wherein the first plate and the second plate are assembled together so that the first plate is positioned against the second plate and the recesses of the first plate communicate with the recesses of the second plate so as to form a plurality of sample rows, a plurality of control rows, and an ink row disposed between the plurality of sample rows and the plurality of control rows, with the plurality of channels of the first plate being disposed between the plurality of control rows and the ink row, and the plurality of channels of the second plate being disposed between the plurality of sample rows and the ink row; and
wherein at least one of the first plate and the second plate is configured to slide relative to the other of the first plate and the second plate in order to form a plurality of sample columns, a plurality of control columns and a plurality of ink columns, with each of the plurality of channels in the second plate being in communication with each of the plurality of sample columns and ink columns and with each of the plurality of channels in the first plate being in communication with each of the plurality of control columns and ink columns;

binding an analyte-specific antibody in at least one recess forming one of the plurality of sample rows of the second plate;

positioning a reagent in a recess adjacent to the sample row containing the analyte-specific antibody, positioning ink in a recess in the ink row, positioning a known concentration of a reactant in a control row, and positioning a reagent in a recess adjacent to the control row containing the known concentration of a reactant;

positioning a sample in the at least one recess containing the analyte-specific antibody;

positioning an analyte-specific antibody comprising a reactant in the at least one recess containing the analyte-specific antibody and the sample;

sliding one of the first plate and the second plate relative to the other of the first plate and the second plate so as to form the plurality of sample columns and control columns, with each sample column being in communication with one of the plurality of channels in the second plate and with each control column being in communication with one of the plurality of channels in the first plate; and

determining the quantity of the target analyte present in the sample by detecting the disposition of the ink contained in the plurality of channels in the second plate and the plurality of channels in the first plate.

In another form of the present invention, there is provided apparatus for determining the quantity of a target analyte present in a sample, the apparatus comprising:

a control recess, a sample recess, an ink recess disposed between the control recess and the sample recess, and a channel fluidically connecting the control recess, the ink recess and the sample recess;

a known concentration of a reactant being disposed in the control recess, an analyte-specific antibody being bound in the sample recess, and ink being disposed in the ink recess;

means for introducing a sample into the sample recess;

means for introducing an analyte-specific antibody comprising a reactant into the sample recess; and

means for introducing a reagent into the sample recess for reacting with the reactant in the sample recess to produce a gas acting on the ink in the ink recess, and means for introducing a reagent into the control recess for reacting with reactant in the control recess to produce a gas also acting on the ink in the ink recess, whereby to enable determination of the quantity of the target analyte present in the sample by detecting the disposition of the ink in the channel.

In another form of the present invention, there is provided a method for determining the quantity of a target analyte present in a sample, the method comprising:

providing apparatus comprising:
a control recess, a sample recess, an ink recess disposed between the control recess and the sample recess, and a channel fluidically connecting the control recess, the ink recess and the sample recess;
a known concentration of a reactant being disposed in the control recess, an analyte-specific antibody being bound in the sample recess, and ink being disposed in the ink recess;

introducing a sample into the sample recess;

introducing an analyte-specific antibody comprising a reactant into the sample recess; and

introducing a reagent into the sample recess for reacting with the reactant in the sample recess to produce a gas acting on the ink in the ink recess, and means for introducing a reagent into the control recess for reacting with reactant in the control recess to produce a gas also acting on the ink in the ink recess;

determining the quantity of the target analyte present in the sample by detecting the disposition of the ink in the channel.

In another form of the present invention, there is provided apparatus for determining the quantity of a target analyte present in a sample, the apparatus comprising:

a first plate comprising a first surface having a first recess containing an analyte-specific antibody and a second recess for containing ink, the first recess being spaced from the second recess;

a second plate comprising a second surface having a third recess for containing a reagent, a fourth elongated recess and a fifth recess, the fifth recess being disposed between, and spaced from, the third recess and the fourth elongated recess;

wherein the first plate and the second plate are assembled together so that the first surface of the first plate faces the second surface of the second plate;

wherein the first plate and the second plate are reconfigurable between (i) a first state in which the first recess is fluidically isolated from the third recess and the fifth recess and the second recess is fluidically isolated from the fourth elongated recess and the fifth recess, and (ii) a second state in which the first recess is fluidically connected to the third recess and the fifth recess and the second recess is fluidically connected to the fourth elongated recess and the fifth recess.

In another form of the present invention, there is provided a method for determining the quantity of a target analyte present in a sample, the method comprising:

providing apparatus comprising:
a first plate comprising a first surface having a first recess containing an analyte-specific antibody and a second recess for containing ink, the first recess being spaced from the second recess;
a second plate comprising a second surface having a third recess for containing a reagent, a fourth elongated recess and a fifth recess, the fifth recess being disposed between, and spaced from, the third recess and the fourth elongated recess;
wherein the first plate and the second plate are assembled together so that the first surface of the first plate faces the second surface of the second plate;
wherein the first plate and the second plate are reconfigurable between (i) a first state in which the first recess is fluidically isolated from the third recess and the fifth recess and the second recess is fluidically isolated from the fourth elongated recess and the fifth recess, and (ii) a second state in which the first recess is fluidically connected to the third recess and the fifth recess and the second recess is fluidically connected to the fourth elongated recess and the fifth recess;

positioning the first plate and the second plate in their first state;

positioning a reagent in the third recess and positioning ink in the second recess;

positioning a sample in the first recess;

positioning an analyte-specific antibody comprising a reactant in the first recess so that the analyte-specific antibody comprising the reactant binds to any analyte present in the sample;

positioning the first plate and the second plate in their second state; and

determining the quantity of the analyte present in the sample by detecting the disposition of the ink in the fourth elongated recess.

### BRIEF DESCRIPTION OF THE DRAWINGS

These and other objects and features of the present invention will be more fully disclosed or rendered obvious by the following detailed description of the invention, which is to be considered together with the accompanying drawings wherein like numbers refer to like parts, and further wherein:

FIG. 1 which shows a typical prior art approach for a protein-based biomarker assay, where a blood sample is drawn from a patient and then processed by a relatively large, complex instrument;

FIG. 2 shows the novel multiplexed volumetric bar chart chip of the present invention;

FIG. 3 shows the novel multiplexed volumetric bar chart chip of FIG. 2 and a barcode scanner which can be used to read the multiplexed volumetric bar chart chip;

FIGS. 4-8 illustrate further details of the novel multiplexed volumetric bar chart chip of the present invention;

FIG. 9 is a schematic drawing of an etching process which can be utilized to form recesses and channels in the top plate and the bottom plate of multiplexed volumetric bar chart chip;

FIG. 10 is a schematic drawing of the assembly and operation of the multiplexed volumetric bar chart chip of the present invention;

FIGS. 11 and 12 are schematic drawings illustrating use of the multiplexed volumetric bar chart chip of the present invention;

FIG. 13 shows the multiplexed volumetric bar chart chip of the present invention prior to the oblique sliding of the top plate relative to the bottom plate;

FIGS. 14-16 show the test results obtained in accordance with the present invention for various samples;

FIGS. 17-20 show specific steps which are performed in accordance with the method of the present invention;

FIGS. 21-32 are a schematic series of views illustrating the assembly and operation of the multiplexed volumetric bar chart chip in one form of the present invention;

FIGS. 33-45 are a schematic series of views showing how, over time, the ink in various bar channels advance in the multiplexed volumetric bar chart chip according to the quantity of target proteins or other types of biomarkers or other molecular analytes present in the sample;

FIG. 46 illustrates specific steps which are performed in accordance with a DNA assay scheme and oxygen generation mechanism;

FIG. 47 shows an alternative embodiment of the novel multiplexed volumetric bar chart chip of the present invention;

FIG. 48 shows images of hydrogen peroxide solution pushed into platinum wells using the multiplexed volumetric bar chart chip of FIG. 47;

FIGS. 49 and 50 show an alternative embodiment of the novel multiplexed volumetric bar chart chip of the present invention;

FIG. 51 shows an alternative embodiment of the novel multiplexed volumetric bar chart chip of the present invention;

FIG. 52 shows an alternative embodiment of the novel multiplexed bar chart chip of the present invention, wherein platinum nanoparticles are utilized in the place of catalase;

FIG. 53 shows an alternative embodiment of the novel multiplexed bar chart chip of the present invention, wherein the channel(s) are arranged in a serpentine configuration;

FIG. 54 shows an alternative embodiment of the novel multiplexed bar chart chip of the present invention, wherein the channels are arranged in straight and V-shaped configurations;

FIGS. 54A and 54B are schematic views showing another alternative form of multiplexed volumetric bar chart chip formed in accordance with the present invention;

FIG. 54C is a schematic view showing further details of the top plate of the multiplexed volumetric bar chart chip of FIGS. 54A and 54B;

FIG. 54D is a schematic view showing further details of the bottom plate of the multiplexed volumetric bar chart chip of FIGS. 54A and 54B;

FIG. 54E is a schematic view of the assembly of the top plate and the bottom plate of the multiplexed volumetric bar chart chip of FIGS. 54A and 54B;

FIG. 54F is a schematic view showing a modified form of the multiplexed volumetric bar chart chip of FIGS. 54A and 54B;

FIGS. 54G-54I are schematic views illustrating the principles of various assays which may be used with the multiplexed volumetric bar chart chip of the present invention;

FIGS. 54J-54M are schematic views illustrating use of the multiplexed volumetric bar chart chip of FIGS. 54A and 54B;

FIG. 54N is a table listing exemplary threshold cutoff values for various analytes which may be assayed using the multiplexed volumetric bar chart chip of FIGS. 54A and 54B;

FIG. 54O is a schematic view showing still another alternative form of multiplexed volumetric bar chart chip formed in accordance with the present invention;

FIG. 54P is a schematic view showing further details of the top plate of the multiplexed volumetric bar chart chip of FIG. 54O;

FIG. 54Q is a schematic view showing further details of the bottom plate of the multiplexed volumetric bar chart chip of FIG. 54O;

FIGS. 54R-54T are schematic views of the assembly of the top plate and the bottom plate of the multiplexed volumetric bar chart chip of FIG. 54O;

FIGS. 54U-54W are schematic views illustrating use of the multiplexed volumetric bar chart chip of FIG. 54O;

FIG. 55 shows an alternative embodiment of the novel multiplexed bar chart chip of the present invention, wherein the novel multiplexed bar chart chip is configured for a hepatocellular carcinoma risk assessment assay;

FIG. 56 shows an alternative embodiment of the novel multiplexed bar chart chip of the present invention, wherein the novel multiplexed bar chart chip is configured for a breast cancer risk/diagnosis assay;

FIG. 57 shows an alternative embodiment of the novel multiplexed bar chart chip of the present invention, wherein the novel multiplexed bar chart chip is configured for a sepsis assessment assay;

FIG. 58 shows an alternative embodiment of the novel multiplexed bar chart chip of the present invention, wherein the novel multiplexed bar chart chip is configured for a drug abuse assessment assay;

FIG. 59 shows an alternative embodiment of the novel multiplexed bar chart chip of the present invention, wherein the novel multiplexed bar chart chip is configured for assay of several important physiological biomarkers; and

FIG. 60 shows an alternative embodiment of the novel multiplexed bar chart chip of the present invention, wherein the novel multiplexed bar chart chip is configured for an assay of DNA, RNA, and/or micro-RNA targets.

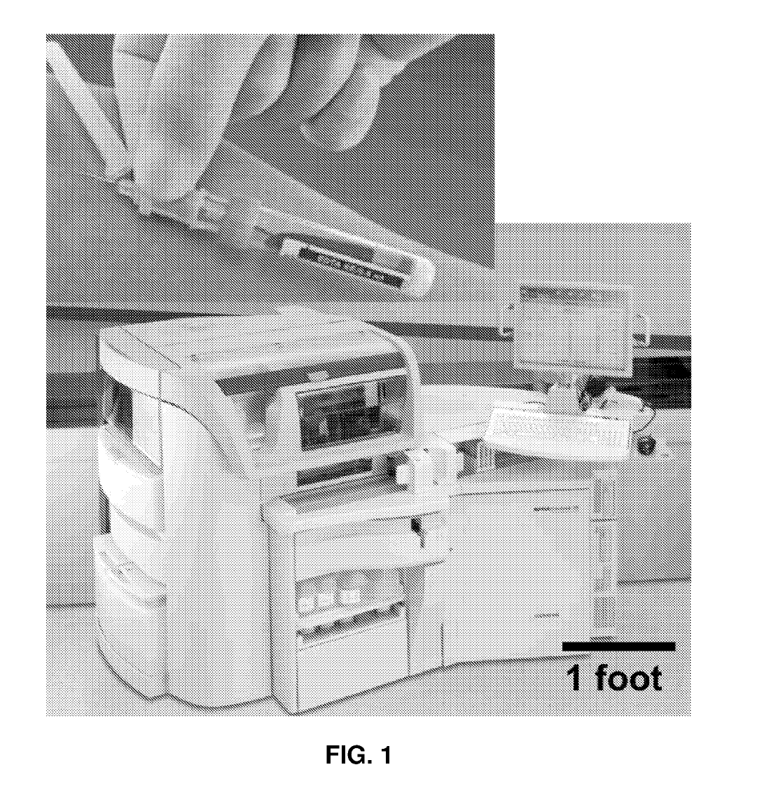

### DETAILED DESCRIPTION OF THE INVENTION

The present invention provides a new method and apparatus for point of care determination of the quantity of a protein (and, preferably, the quantity of multiple proteins) present in a sample.

### Multiplexed Volumetric Bar Chart Chip

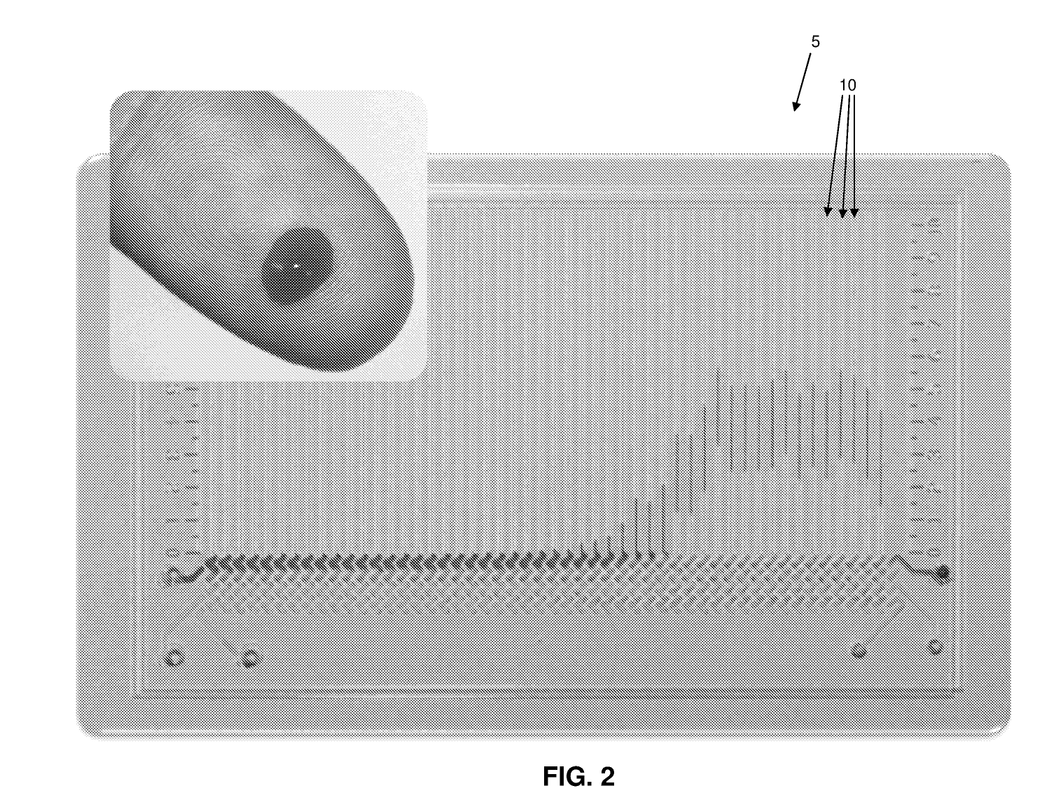

More particularly, and looking now at FIG. 2, in one preferred form of the invention, there is provided a novel multiplexed volumetric bar chart chip 5. Multiplexed volumetric bar chart chip 5 is configured to simultaneously determine the quantity of multiple proteins which may be present in a sample, with the quantity of each protein which is present in the sample being indicated in a particular one of a plurality of bar channels 10. By way of example but not limitation, 6, 10, 30, and 50-plexed, or more than 50-plexed, channels may be incorporated into multiplexed volumetric bar chart chip 5. Bar channels 10 may be straight (as shown in FIG. 2) or curved (e.g., serpentine, circular, z-shaped) or formed in any other configuration which provides a series of channels having a length. As a result of this construction, the review of a particular bar channel 10 will indicate the quantity of a particular protein which may be present in the sample and, significantly, the collective array of the plurality of bar channels 10 will simultaneously indicate, in bar chart form, the quantities of multiple proteins which may be present in the sample, whereby to provide multi-protein quantity measurements and hence a more comprehensive diagnostic result.

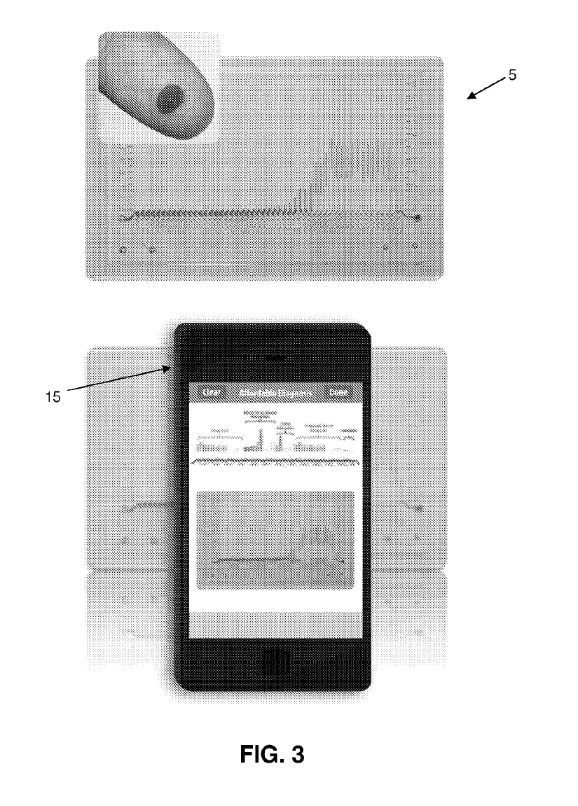

As seen in FIG. 3, the multi-protein measurements presented in bar chart form by multiplexed volumetric bar chart chip 5 may then be read with a smart-phone or barcode scanner 15, whereby to automate the data collection process.

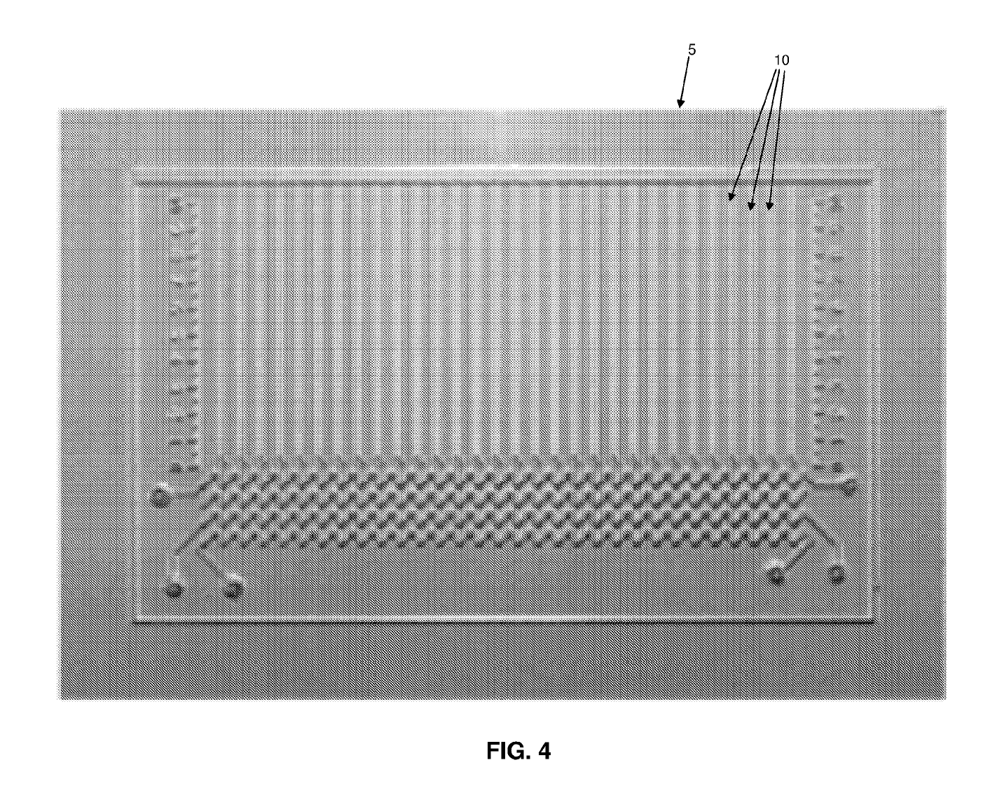

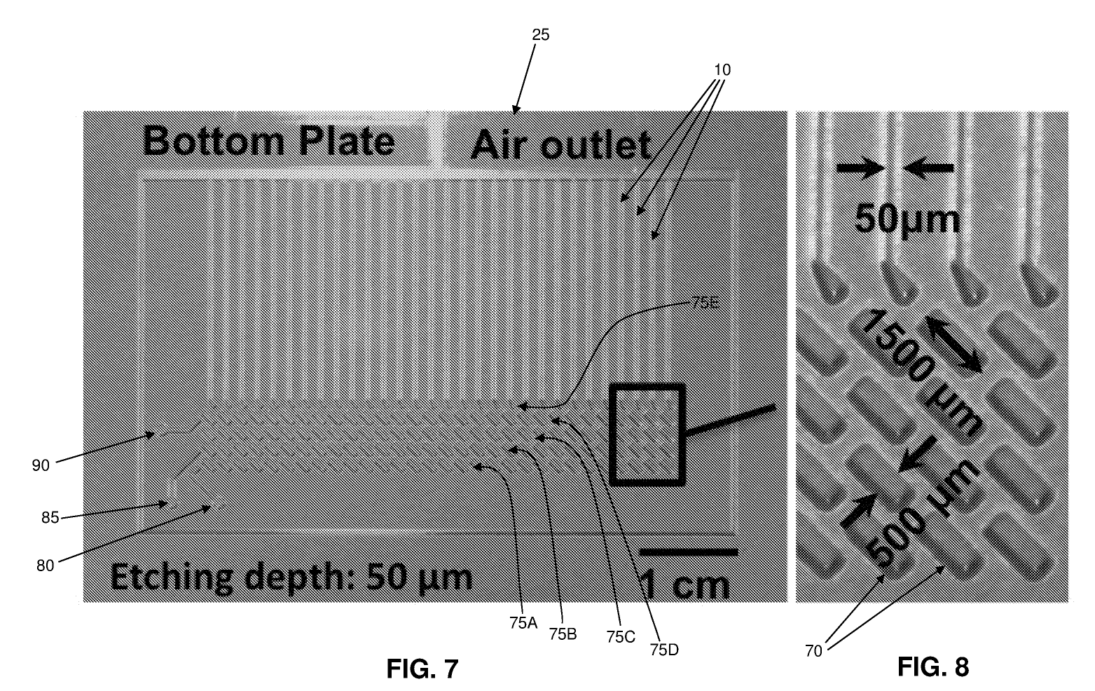

Looking now at FIGS. 4-8, multiplexed volumetric bar chart chip 5 comprises two plates, a transparent top plate 20 and a bottom plate 25 (which may or may not be transparent).

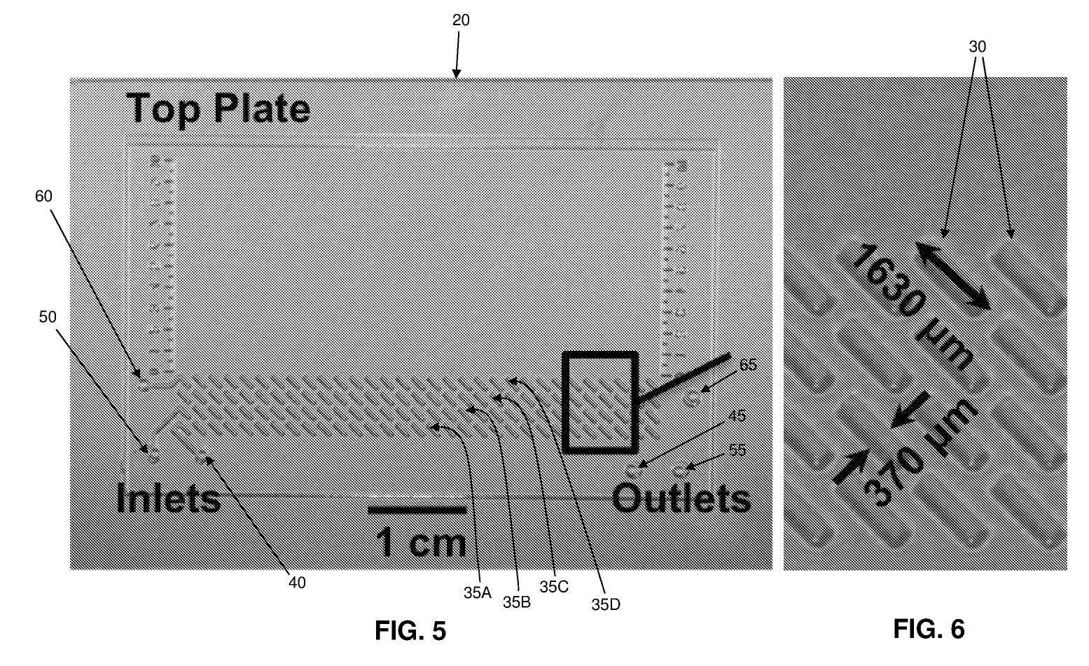

Top plate 20 (FIGS. 5 and 6) has a plurality of recesses 30 formed on its bottom surface, with recesses 30 being arranged in a plurality of rows 35 (i.e., 35A, 35B, 35C, etc.), with each of the recesses 30 extending at a 45 degree angle relative to the axis of a given row 35, and with a recess 30 in one row 35 being aligned with an offset recess 30 in an adjacent row 35. An inlet 40 is connected to a far side recess 30 on the ultimate row 35A, and an outlet 45 is formed adjacent to the opposite far side recess 30 on the same ultimate row 35A. An inlet 50 is connected to a far side recess 30 on the penultimate row 35B, and an outlet 55 is formed adjacent to the opposite far side recess 30 on the same penultimate row 35B. The antepenultimate row 35C lacks both an inlet and an outlet. An inlet 60 is connected to a far side recess 30 on the ante-antepenultimate row 35D, and an outlet 65 is formed adjacent to the opposite far side recess 30 on the same ante-antepenultimate row 35D.

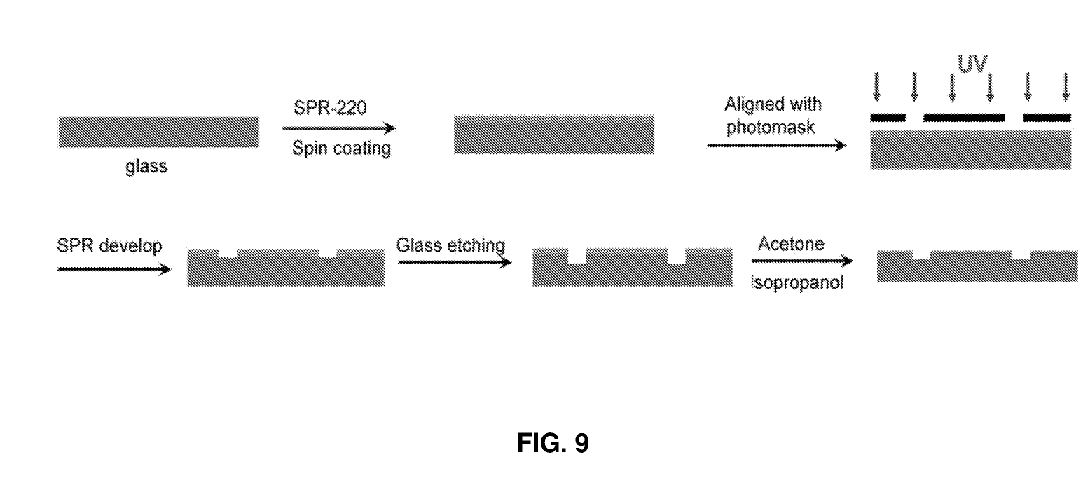

In one preferred form of the invention, and looking now at FIG. 9, recesses 30, inlets 40, 50, 60, and outlets 45, 55, 65 are all formed in the bottom surface of top plate 20 using a conventional etching process of the sort well known in the etching arts. Preferably, recesses 30, inlets 40, 50, 60 and outlets 45, 55, 65 are etched in the bottom surface of a glass plate. Alternatively, recesses 30, inlets 40, 50, 60 and outlets 45, 55, 65 may be formed in a silicon plate, a plastic plate, a ceramic plate, a quartz plate, a metal oxide plate or other appropriate substrate material.

Bottom plate 25 has a plurality of recesses 70 formed on its top surface, with recesses 70 being arranged in a plurality of rows 75 (i.e., 75A, 75B, 75C, etc.), with each of the recesses 70 extending at a 45 degree angle relative to the axis of a given row 75, and with a recess 70 in one row 75 being aligned with an offset recess 70 in an adjacent row 75. An outlet 80 is connected to a far side recess 70 on the ultimate row 75A. An outlet 85 is connected to a far side recess 70 on the penultimate row 75B. The antepenultimate row 75C lacks an outlet. An outlet 90 is connected to a far side recess 70 on the ante-antepenultimate row 75D. In addition, the plurality of bar channels 10 are formed on the top surface of bottom plate 25, with each of the bar channels 10 being connected to a recess 70 in the ante-ante-antepenultimate row 75E (see FIG. 8), and with each of the bar channels 10 extending parallel to one another and perpendicular to the axis of rows 75.

In one preferred form of the invention, and looking now at FIG. 9, recesses 70, outlets 80, 85, 90, and bar channels 10 are all formed in the top surface of bottom plate 25 using a conventional etching process of the sort well known in the etching arts. Preferably, recesses 70, outlets 80, 85, 90, and bar channels 10 are etched in the top surface of a glass plate. Alternatively, recesses 70, outlets 80, 85, 90, and bar channels 10 may be formed in a silicon plate, a plastic plate, a ceramic plate, a quartz plate, a metal oxide plate or other appropriate substrate material.

### Assembly of Multiplexed Volumetric Bar Chart Chip

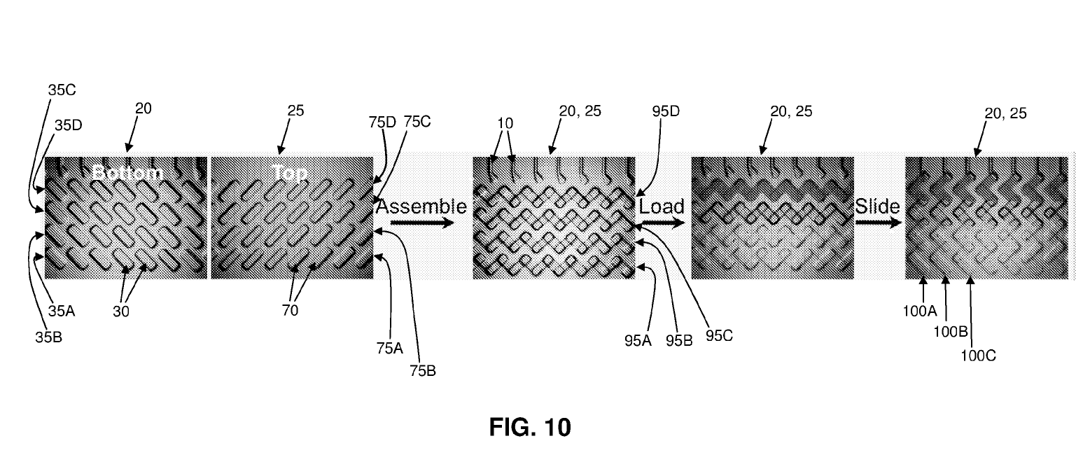

Looking next at FIG. 10, top plate 20 is assembled on top of bottom plate 25 so that recesses 30 in top plate 20 communicate with recesses 70 in bottom plate 25. More particularly, when top plate 20 is assembled on top of bottom plate 25 in this manner, recesses 30 in top plate 20 will cooperate with recesses 70 in bottom plate 25 so as to initially form a plurality of continuous rows 95 (i.e., 95A, 95B, 95C, 95D, etc.) in multiplexed volumetric bar chart chip 5, with the inlet 40 of ultimate row 95A being connected with the outlet 45 of ultimate row 95A, with the inlet 50 of the penultimate row 95B being connected with the outlet 55 of the penultimate row 95B, and with the inlet 60 of the ante-antepenultimate row 95D being connected with the outlet 65 of the ante-antepenultimate row 95D. As noted above, the antepenultimate row 95C lacks both an inlet and an outlet.

Still looking now at FIG. 10, it will be appreciated that, due to the dispositions of recesses 30 in top plate 20 and recesses 70 in bottom plate 25, an oblique slide of top plate 20 relative to bottom plate 25 disrupts the aforementioned rows 95 and causes them to transform into a plurality of continuous columns 100 (i.e., 100A, 100B, 100C, etc.), with each column 100 being in fluid communication with one of the aforementioned bar columns 10.

### Determining the Quantity of Multiple Proteins Present in a Sample Using the Multiplexed Volumetric Bar Chart Chip

In view of the foregoing construction, multiplexed volumetric bar chart chip 5 can be used to simultaneously determine the quantity of multiple proteins present in a sample, with the quantity of each specific protein being indicated in a particular one of the plurality of bar channels 10.

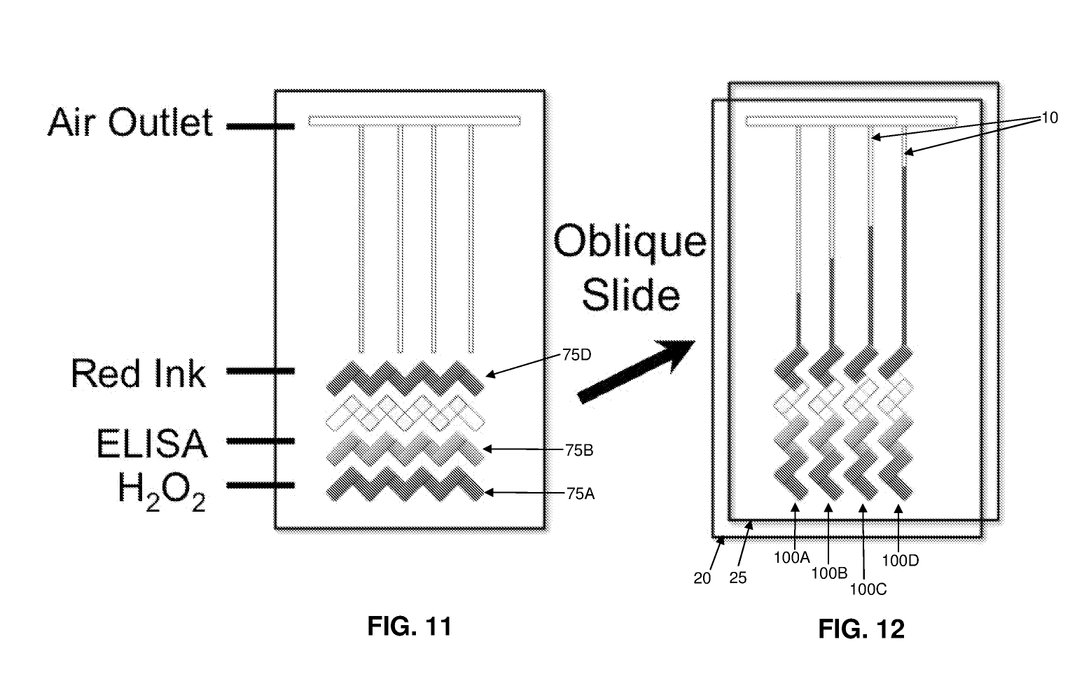

More particularly, and referring now to FIGS. 11 and 12, and as will hereinafter be discussed in further detail below, during manufacture of multiplexed volumetric bar chart chip 5, a different protein-specific antibody is bonded in a recess 30 of the penultimate row 35B. As a result, after the bottom plate 20 and top plate 25 are assembled together, row 75B will contain a series of different protein-specific antibodies, with a different protein-specific antibody being located in each recess 30 of the row 75B.

Prior to use, hydrogen peroxide (H₂O₂) is introduced into inlet 40 of multiplexed volumetric bar chart chip 5, whereby to fill the ultimate row 75A of multiplexed volumetric bar chart chip 5 with hydrogen peroxide. Red ink (or some other colored material which is readily discernible through top plate 25 and against bottom plate 20) is introduced into inlet 60 of multiplexed volumetric bar chart chip 5, whereby to fill the ante-antepenultimate row 75D of multiplexed volumetric bar chart chip 5 with red ink. Antepenultimate row 75C is intentionally left blank to serve as an air spacer, thereby avoiding direct contact between a sample and the red ink.

Then, when a sample is to be checked for the presence and/or quantity of specific proteins (i.e., the proteins which will bind to the protein-specific antibodies already bound to the recesses 30 of row 75B), the sample is introduced into inlet 50 of multiplexed volumetric bar chart chip 5 so that the sample fills the penultimate row 75B. This action causes the sample to mix with the different protein-specific antibodies which are bonded to bottom plate 20 in the recesses 30, so that the target proteins bind to the appropriate protein-specific antibodies in the recesses 30. Significantly, each target protein binds to only one protein-specific antibody, and such binding takes place in only one of the recesses 30 in the penultimate row 75B. Thereafter, the penultimate row 75B is flushed so as to remove any materials which are not bound to a protein-specific antibody.

Next, catalase is introduced into inlet 50 of multiplexed volumetric bar chart chip 5 so as to fill the penultimate row 75B. This action causes the catalase to bind to the target proteins which are themselves bound to the protein-specific antibodies in the recesses 30. It will be appreciated that, to this end, the catalase is a mixture of all the catalase detecting probes required for binding to the target proteins (e.g., silica nanoparticles conjugated with detecting antibodies and catalase molecules). Then excess catalase is rinsed from the penultimate row 75B.

Thereafter, top plate 25 is slid obliquely relative to bottom plate 20, causing rows 75 (i.e., 75A, 75B, 75C, 75D, etc.) to be disrupted and transformed into columns 100 (i.e., 100A, 100B, 100C, etc.). As this row-to-column transformation occurs, each recess 30 (containing the protein-specific antibodies and any target proteins bound thereto and any catalase bound thereto) previously located in penultimate row 75B becomes incorporated as a section of a specific column 100 (i.e., 100A, 100B, 100C, etc.). In addition, as this row-to-column transformation occurs, the hydrogen peroxide contained in row 75A is permitted to advance up each of the columns 100 and thereby mix with any catalase bound to the target proteins (which are themselves bound to the protein-specific antibodies), the mixing of which causes a reaction which releases oxygen gas. The oxygen gas is produced in proportion to the quantity of catalase present in a given column (and hence in proportion to the quantity of target proteins which are present in a given column). Thus, the quantity of oxygen gas produced in a given column 100 is proportional to the quantity of target proteins which are present in a given column 100, with each of the columns 100 containing a different target protein (by virtue of the fact that each of the columns 100 contains a different protein-specific antibody). The oxygen gas produced by the reaction accumulates within the limited volume of columns 100 and causes an increase in pressure, which propels the red ink contained in columns 100 into and along bar columns 10, with the ink advancing a distance along bar columns 10 which is proportional to the quantity of oxygen gas produced in that column, which is in turn proportional to the quantity of the target proteins which are bound to the protein-specific antibodies disposed in the recesses associated with that column.

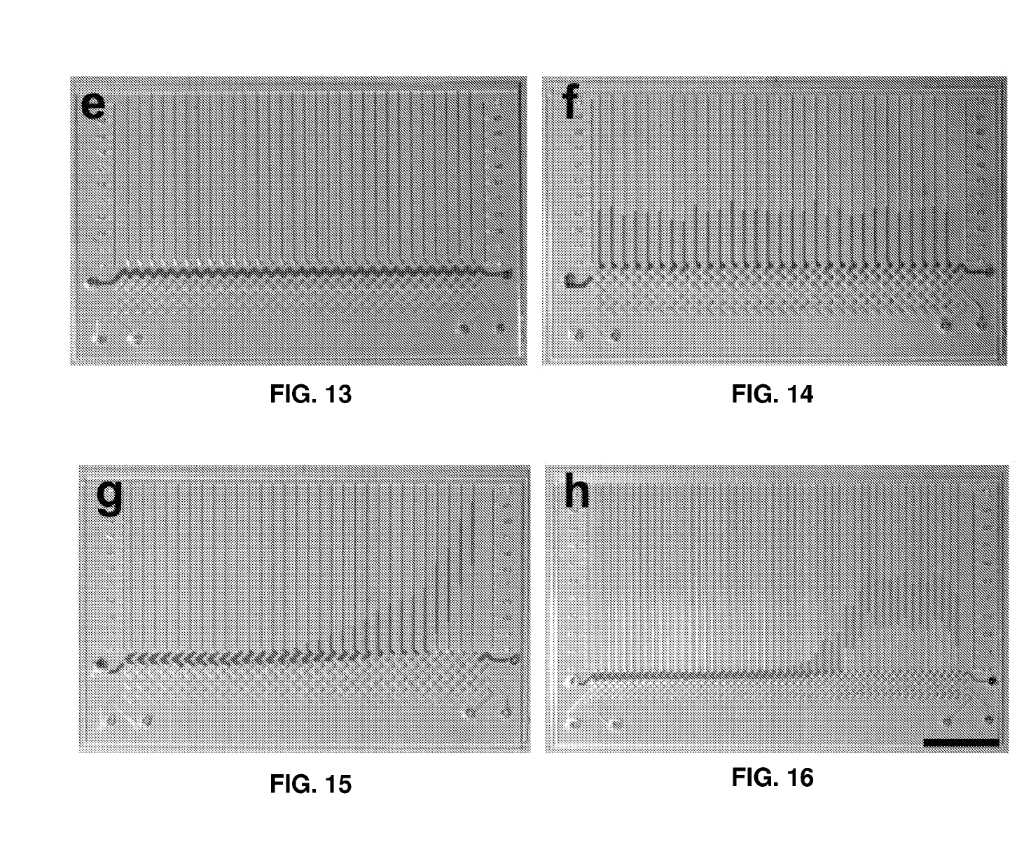

As a result of the foregoing, by disposing different protein-specific antibodies in different ones of the recesses 30 of rows 35 of bottom plate 20, multiplexed volumetric bar chart chip 5 can be used to simultaneously determine the quantity of multiple proteins present in a sample, with the quantity of each protein being indicated in a particular one of a plurality of bar channels 10. See, for example, FIGS. 13-16, where FIG. 13 shows multiplexed volumetric bar chart chip 5 prior to the oblique sliding of top plate 25 relative to bottom plate 20, and FIGS. 14-16 show the test results for various samples.

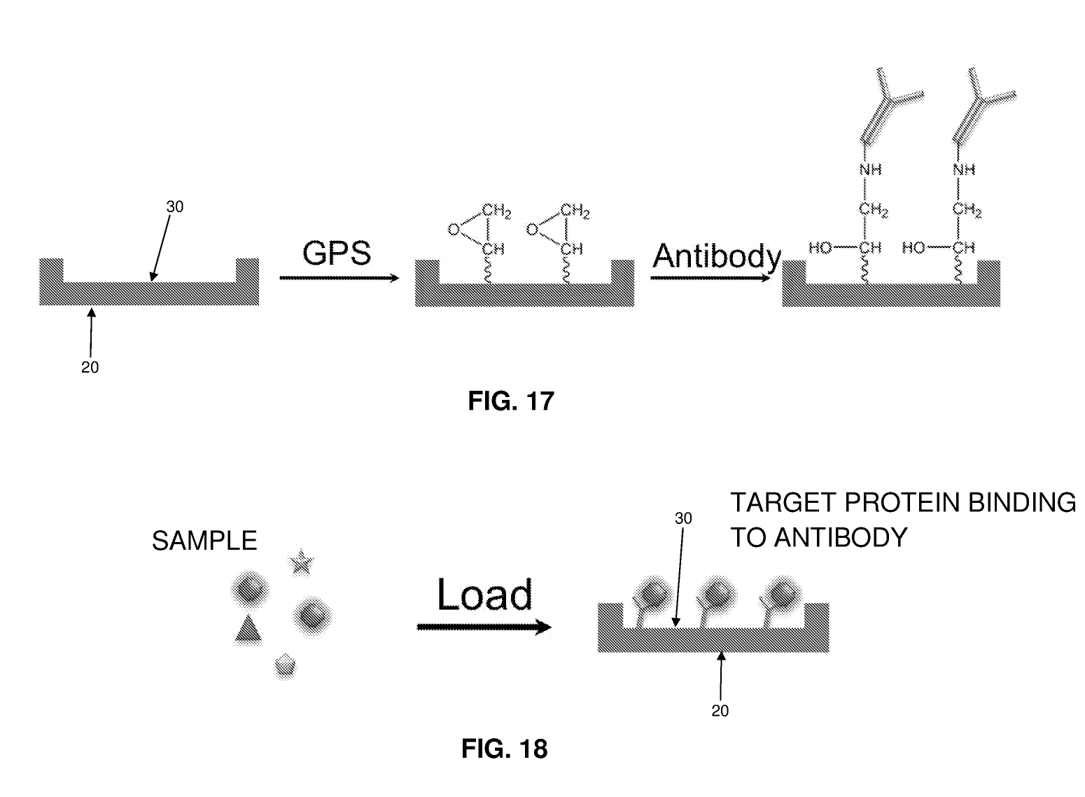

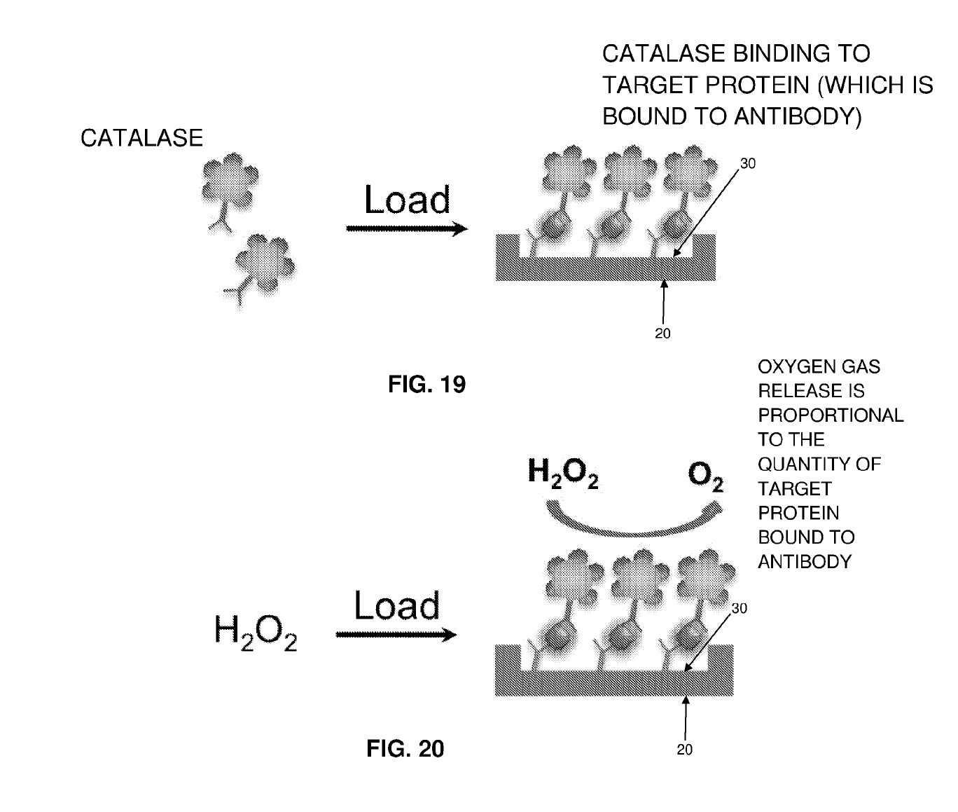

FIGS. 17-20 show specific steps in the foregoing process. Specifically, FIG. 17 shows a protein-specific antibody being bound in a recess 30 of bottom plate 20; FIG. 18 shows a sample being loaded into a recess 30 of bottom plate 20, whereby to bind a target protein to a protein-specific antibody; FIG. 19 shows catalase being loaded into a recess 30 so as to bind catalase to a target protein (which is itself bound to a protein-specific antibody); and FIG. 20 shows hydrogen peroxide being loaded into a recess 30, whereby to release oxygen gas in proportion to the quantity of target protein present in a recess 30.

If desired, the same protein-specific antibody can be bound in multiple recesses 30 of penultimate row 35B of bottom plate 20, whereby to provide redundancy.

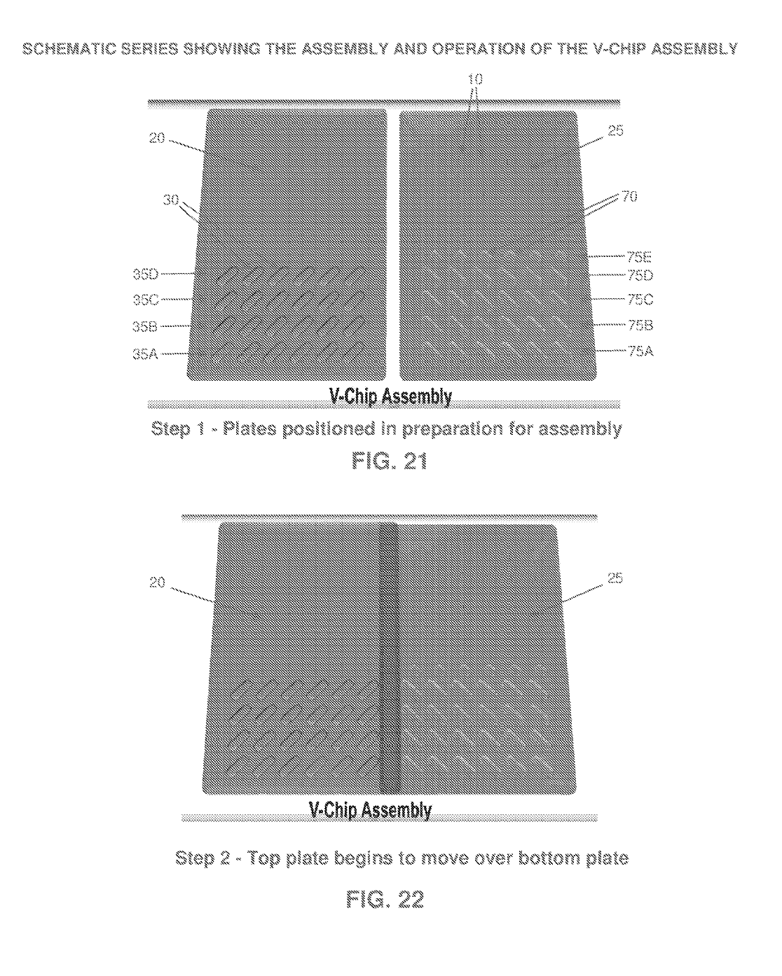

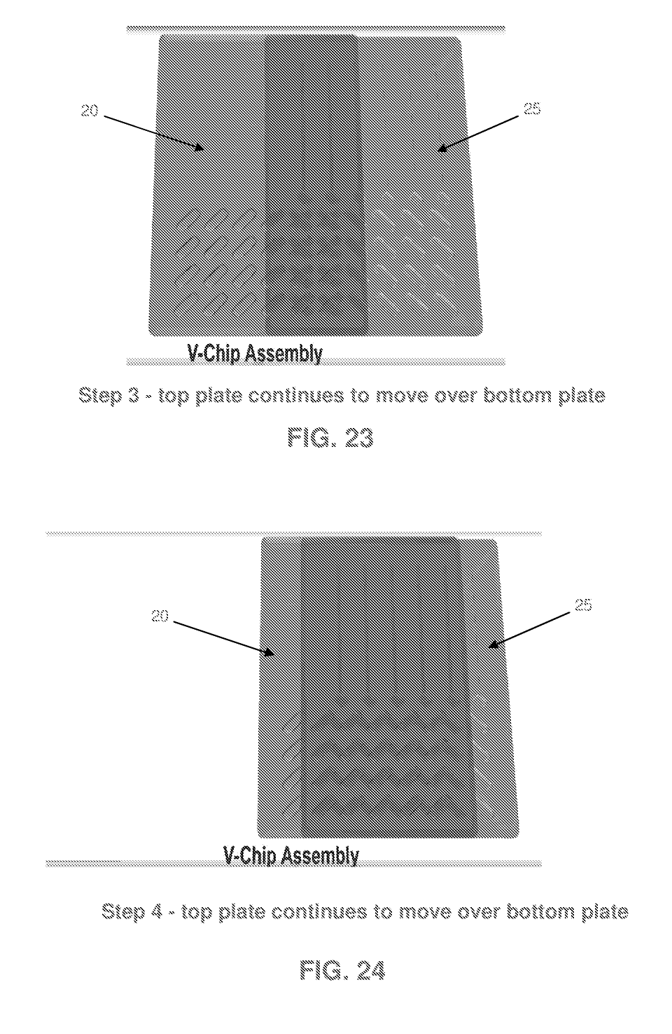

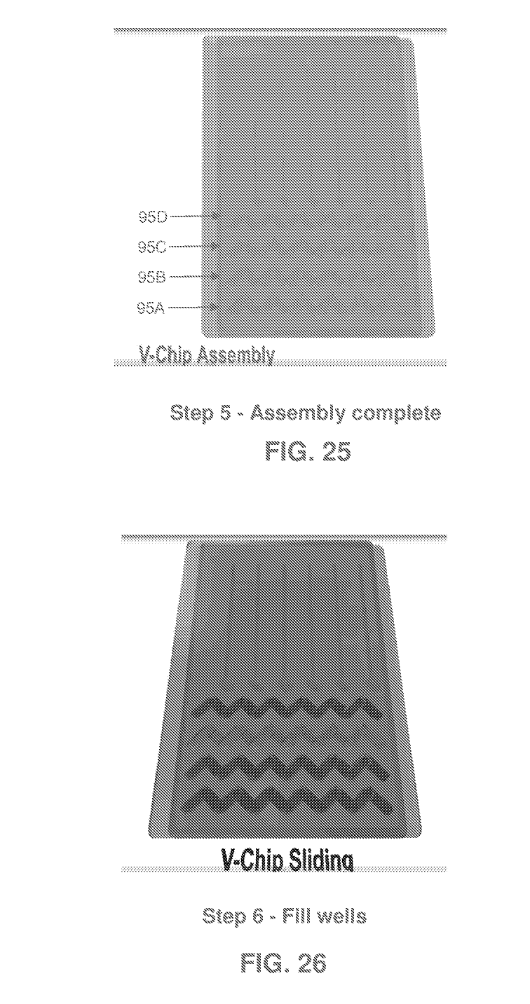

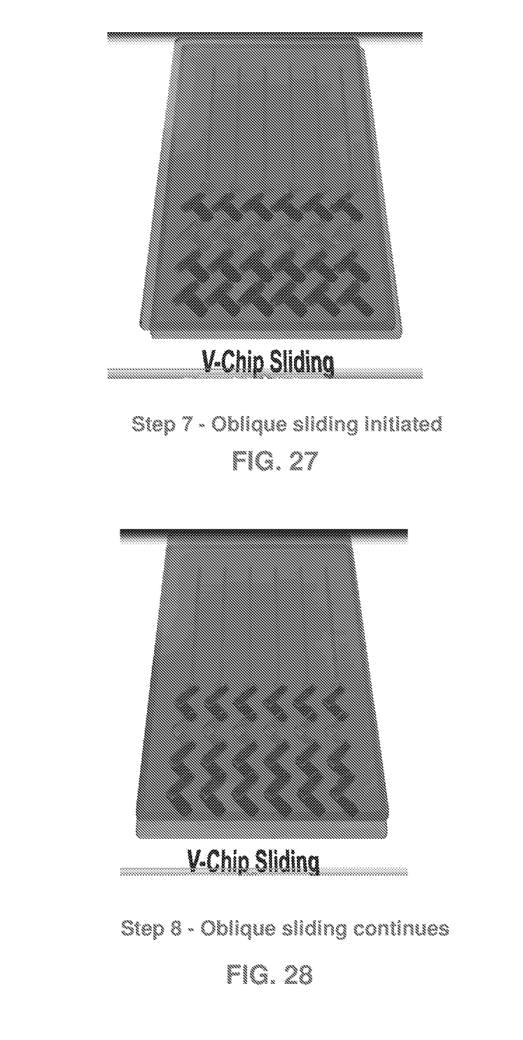

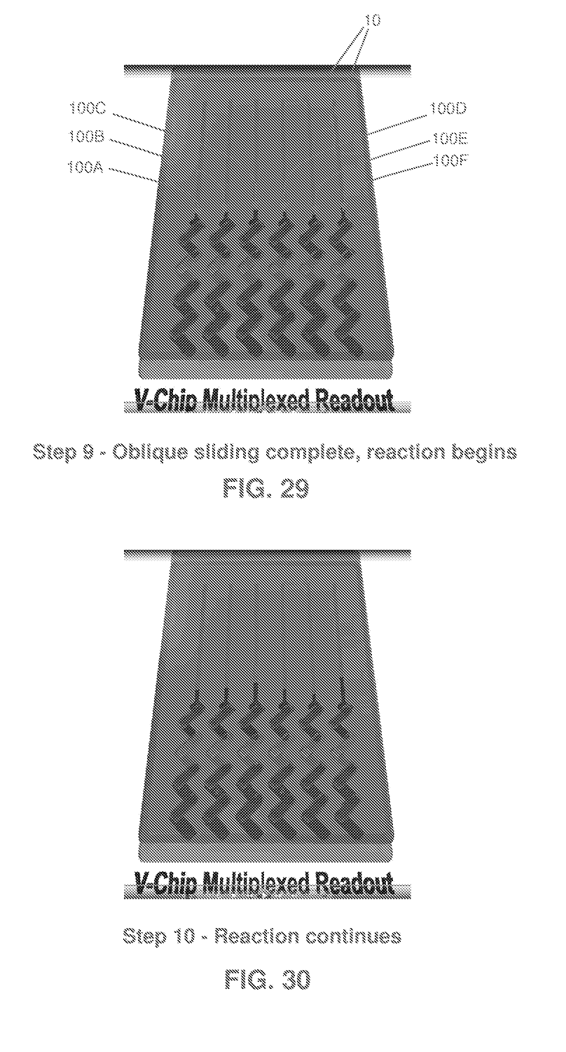

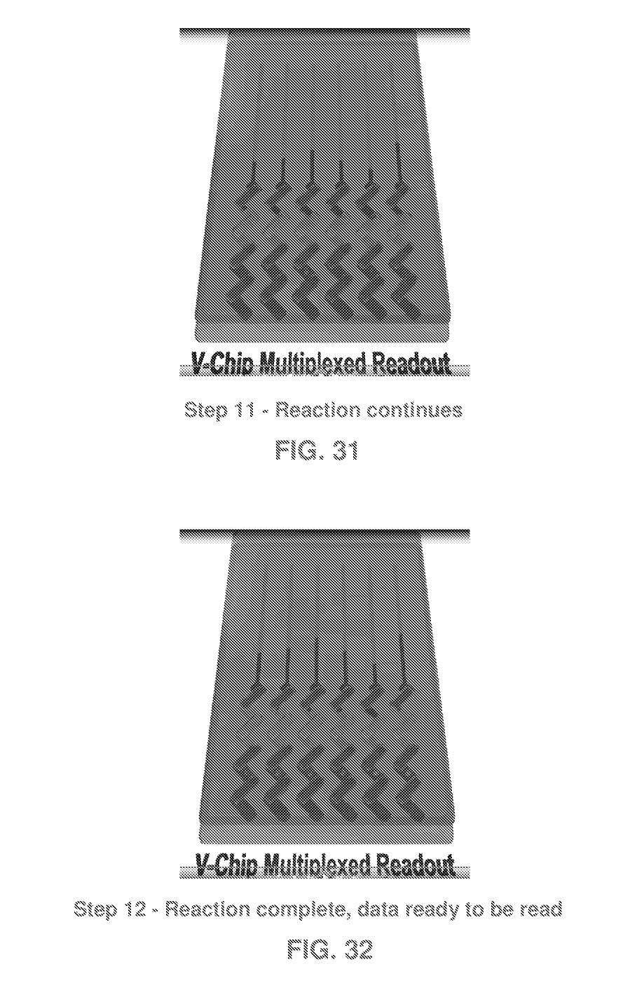

FIGS. 21-32 are a schematic series of views showing the assembly and operation of the multiplexed volumetric bar chart chip in one preferred form of the present invention.

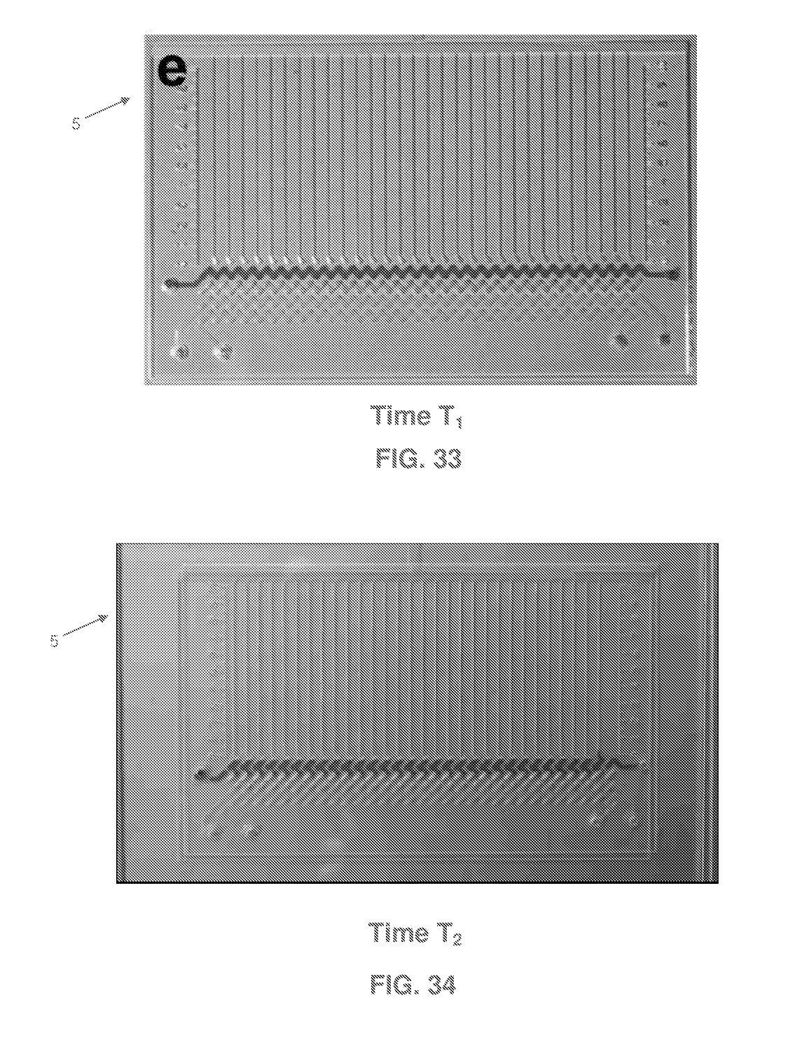

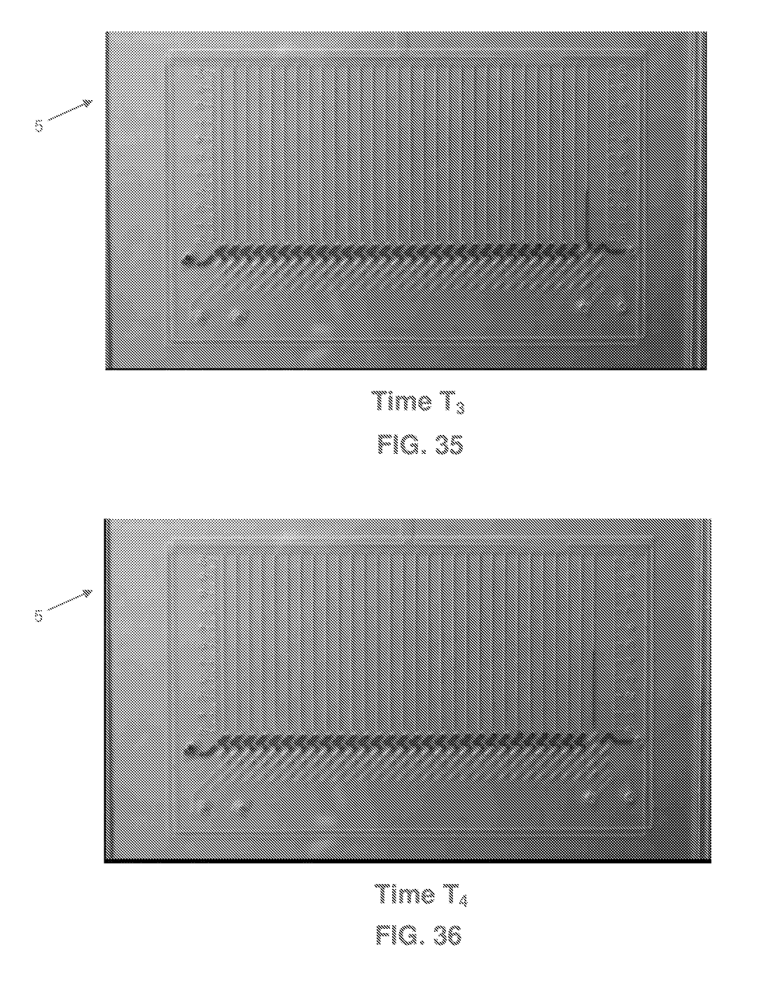

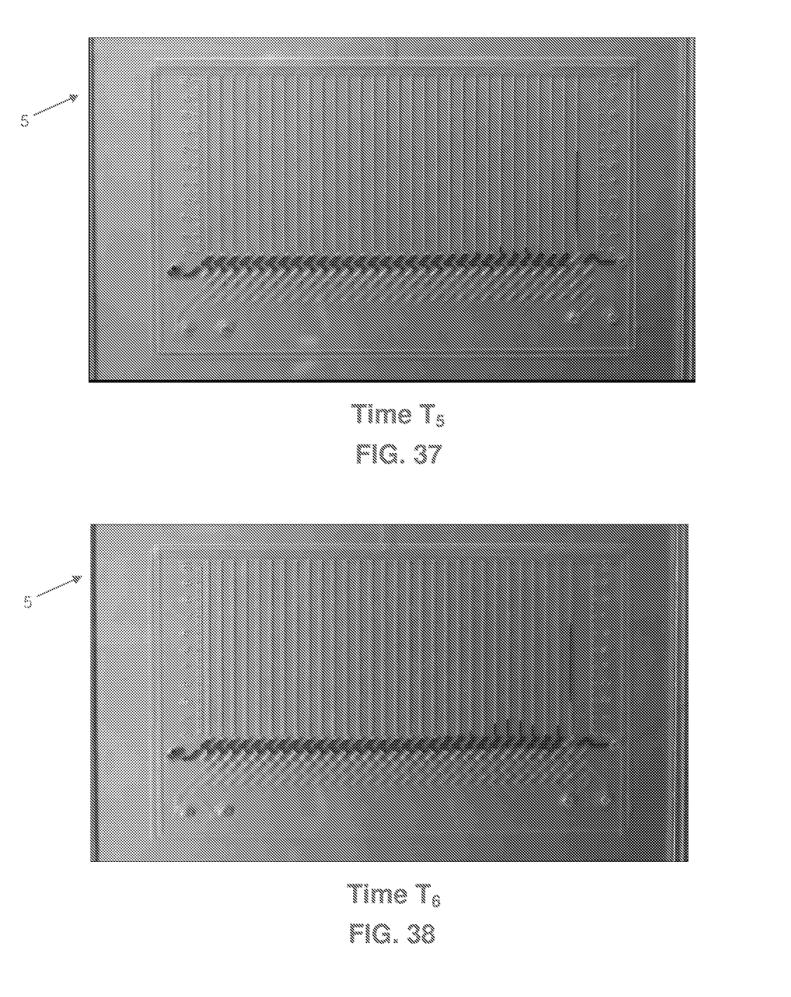

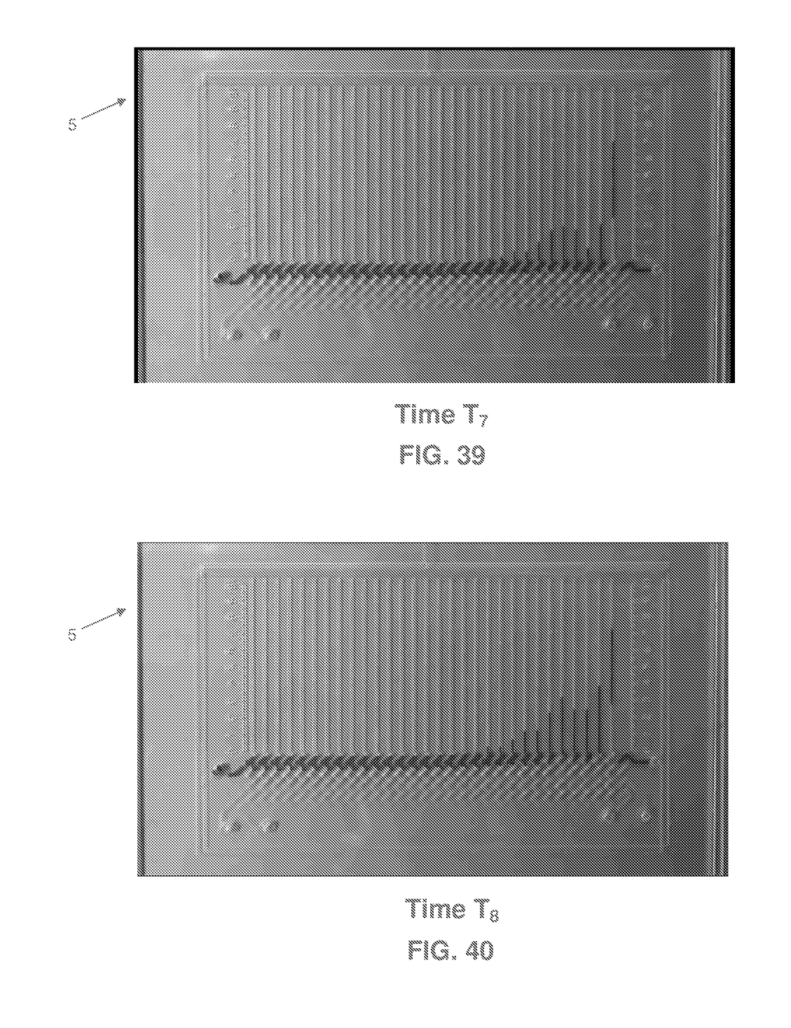

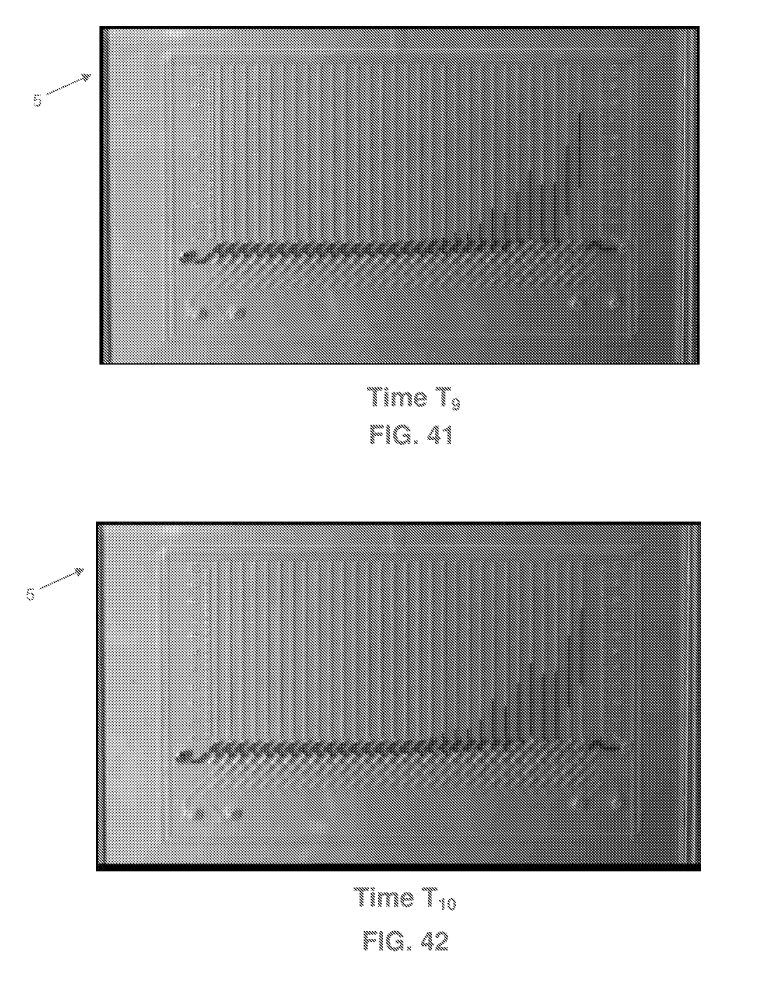

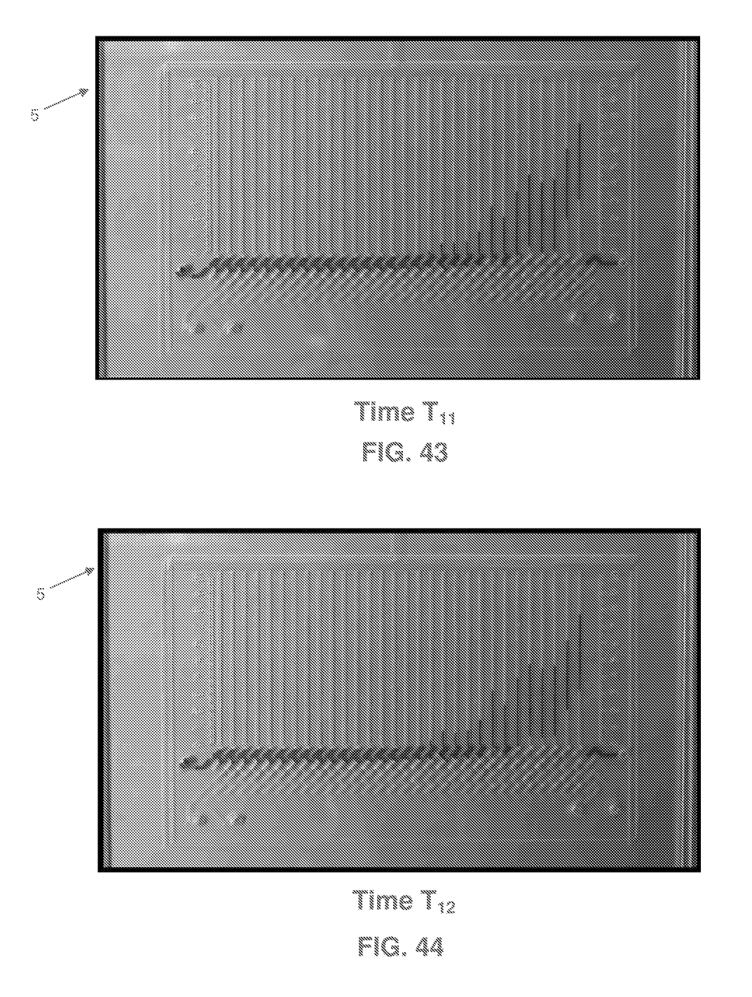

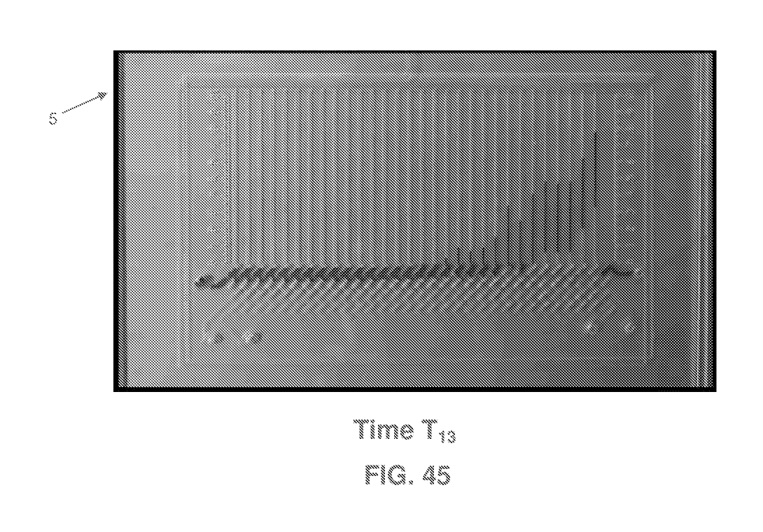

FIGS. 33-45 are a schematic series of views showing how, over time, the ink in a given bar channel advances a distance along that bar channel which is proportional to the quantity of the target protein which are bound to the protein-specific antibody disposed in the recess associated with that bar channel, whereby to indicate, in multiplexed volumetric bar chart form, the results of a simultaneous multi-protein assay.

The novel method and apparatus of the present invention provides instant and visual quantitation of target biomarkers or other molecular analytes and provides a visualized bar chart without the use of instruments, data processing or graphic plotting. Thus, since the novel method and apparatus of the present invention does not require the use of complex instruments, the novel method and apparatus of the present invention can be easily used as a point of care determination of the quantity of a protein (and, preferably, the quantity of multiple proteins) present in a sample. More particularly, the novel method and apparatus of the present invention can be used as a point of care determination of the quantity of protein, nucleic acid, peptide, sugar, organic compounds, polymer, metal ions, and/or other molecular analytes, as well as the quantity of bacteria, cells, and/or particles.

### Alternative Probes and/or Reagents

In the foregoing description, gas is generated by the reaction of an ELISA probe with a reagent, and specifically, gas is generated by the reaction of the ELISA probe (i.e., the protein-specific antibody which is bound to the target protein which is bound to the catalase) with hydrogen peroxide. It is important to note that many other combinations of a probe and a reagent may be used to generate gas. By way of example but not limitation, such probe and reagent combination may include catalase and hydrogen peroxide, platinum film or particles and hydrogen peroxide, catalase and carbamide peroxide, zinc and chloric acid, iron and chloric acid, and other similar combinations. Thus, since the multiplexed volumetric bar chart chip readout is based on the volumetric measurement of a gas generation, many fast responsive gas generation schemes can be used for the system, including catalase with hydrogen peroxide, platinum film or particles and hydrogen peroxide, catalase and carbamide peroxide, zinc and chloric acid, iron and chloric acid, and other similar combinations.

Furthermore, the multiplexed volumetric bar chart chip is based on a sandwich assay. In the foregoing description, a capture antibody binds to an analyte and a detecting antibody conjugated with a catalase probe indicates the amount. Thus, the sandwich scheme is made up of capture antibody/analyte/detecting antibody conjugated with a catalase probe.

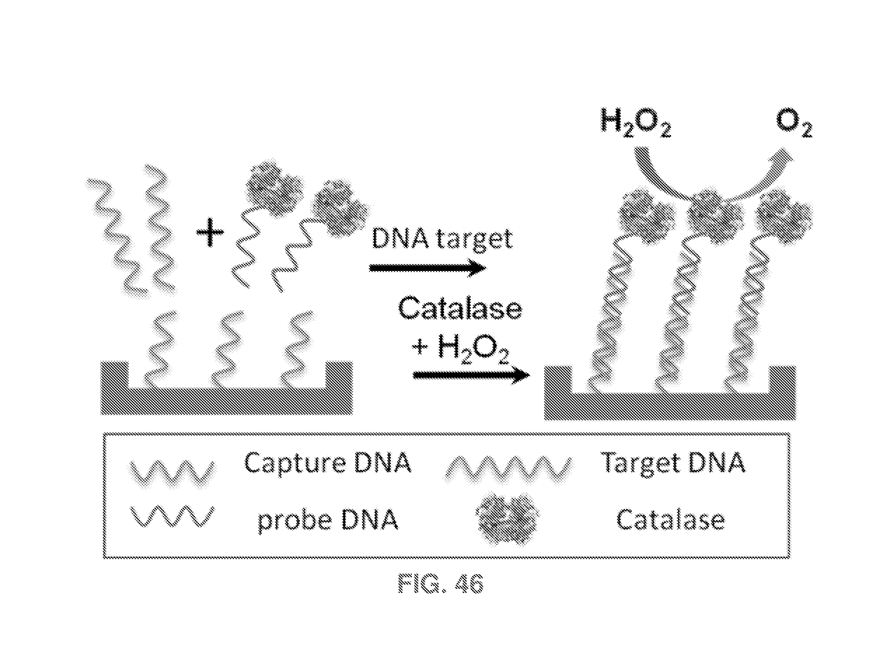

This type of sandwich scheme could also be extended to nucleic acid hybridization, where the sandwich is capture DNA strand/target strand/detecting DNA strand (i.e., the target strand has a first half complimentary to the capture DNA strand and a second half complimentary to the detecting DNA strand). By way of example but not limitation, see FIG. 46, which shows specific steps that are performed in accordance with a DNA assay scheme and oxygen generation mechanism.

Additionally, this type of sandwich scheme could also be extended to hydrogen bonding, electrostatic reaction or interaction, or covalent bonding, where the target analyte is captured by a surface with a coating that can adhere the analyte by either hydrogen bonding, electrostatic reaction or interaction or the formation of a covalent bond. The readout of the adhered or bonded analyte can then be detected by the detecting antibody with a catalase probe. The sandwich of these types are surfaces (with adhesion forces of hydrogen bonding, electrostatic interaction or covalent bonding)/analyte/probe of detecting antibody with catalase.

### Alternative Embodiment of Multiplexed Volumetric Bar Chart Chip Utilizing Amplification Cascades

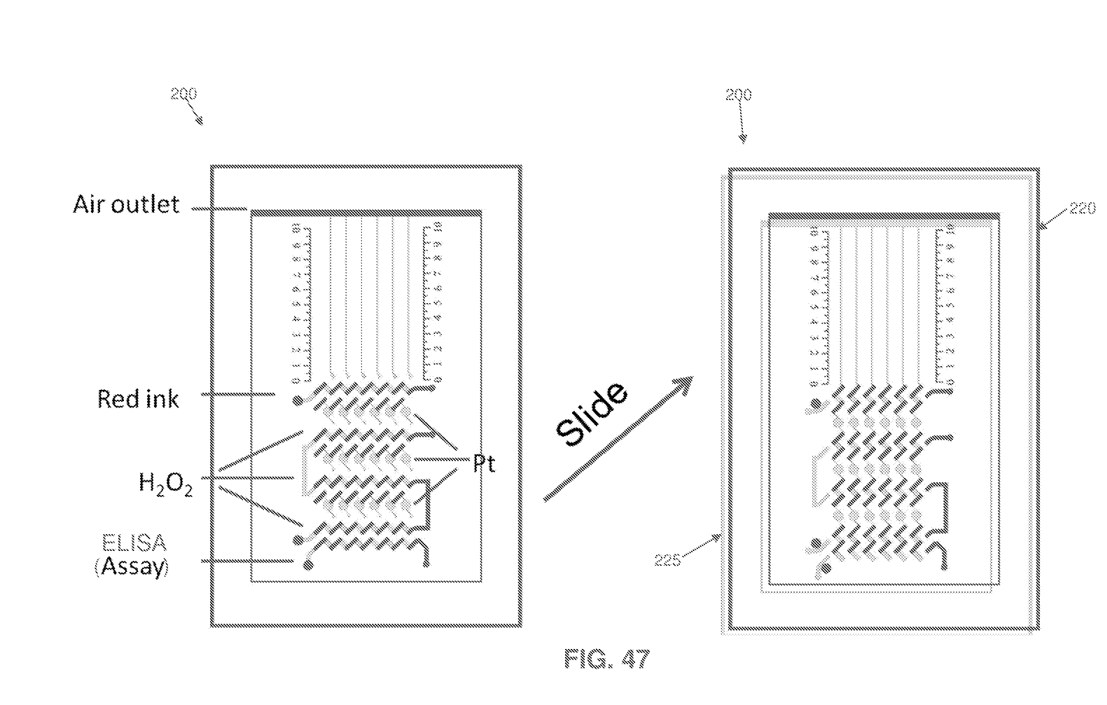

In another embodiment of the present invention, and looking now at FIG. 47, a novel multiplexed volumetric bar chart chip 200 is provided which may be used in accordance with the present invention to determine the quantity of a target protein or other types of biomarkers or other analytes, wherein the signal for determining the quantity of the target protein or other types of biomarkers or other analytes is amplified.

More particularly, multiplexed volumetric bar chart chip 200 comprises two glass plates, a transparent top plate 220 and a bottom plate 225 (which may or may not be transparent).

Top plate 220 and bottom plate 225 are similar to top plate 20 and bottom plate 25 discussed above, except that the plurality of rows are arranged on the multiplexed volumetric bar chart chip 200 so that the recesses in the rows are filled with the ELISA reagents (Assay) (i.e., the protein-specific antibody, with the sample and catalase bound thereto), hydrogen peroxide, platinum film, hydrogen peroxide, platinum film, hydrogen peroxide, platinum film and ink.

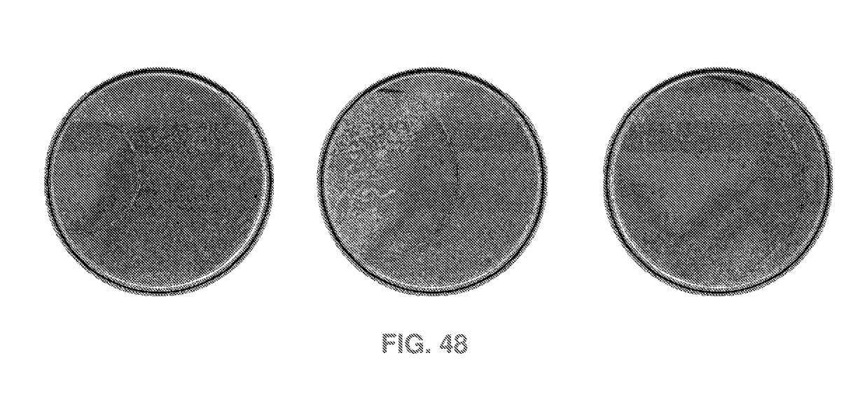

As the ELISA reagent reacts with the hydrogen peroxide, oxygen is generated, with that oxygen being proportional to the quantity of the target antibody present in the sample. The oxygen generated by the ELISA reaction in turn drives a quantity of unreacted hydrogen peroxide (that is proportional to the quantity of oxygen produced from the ELISA reaction) into the next row of the chip (which contains platinum film). When this unreacted hydrogen peroxide passes into the row containing the platinum film, additional oxygen is generated, with the quantity of oxygen generated being proportional to (but greater than) the quantity of oxygen produced from the original ELISA reaction). This process cascades down the successive rows of the chip and, with each step, the amount of oxygen produced is proportional to (but successively greater than) the original quantity of oxygen produced by the ELISA reaction, which is in turn proportional to the quantity of the target protein or other types of biomarkers or other analytes present in the sample. However, since more oxygen is produced by each successive hydrogen peroxide/platinum film reaction, the signal (i.e., the advancement of the red ink in the plurality of channels) is amplified. Since the advancement of the red ink is the sum of the catalase reacting with hydrogen peroxide and the results of the platinum film reacting with hydrogen peroxide over three steps, multiplexed volumetric bar chart chip 200 exhibits a higher sensitivity than the multiplexed volumetric bar chart chip 5 discussed above. See, for example, FIG. 48, which shows images of hydrogen peroxide solution being pushed into successive platinum wells. Due to the accumulated volume of oxygen at different stages of the chip, more hydrogen peroxide was pushed into the platinum wells at the higher stage than at the lower stage.

### Alternative Embodiment of Multiplexed Volumetric Bar Chart Chip Utilizing Pre-Loaded Reagents

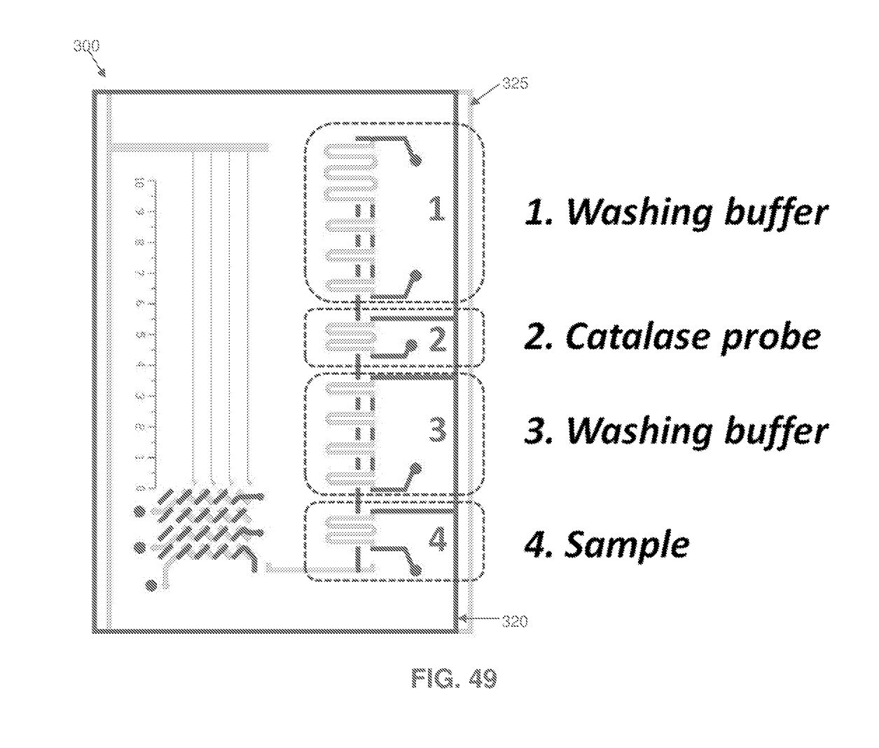

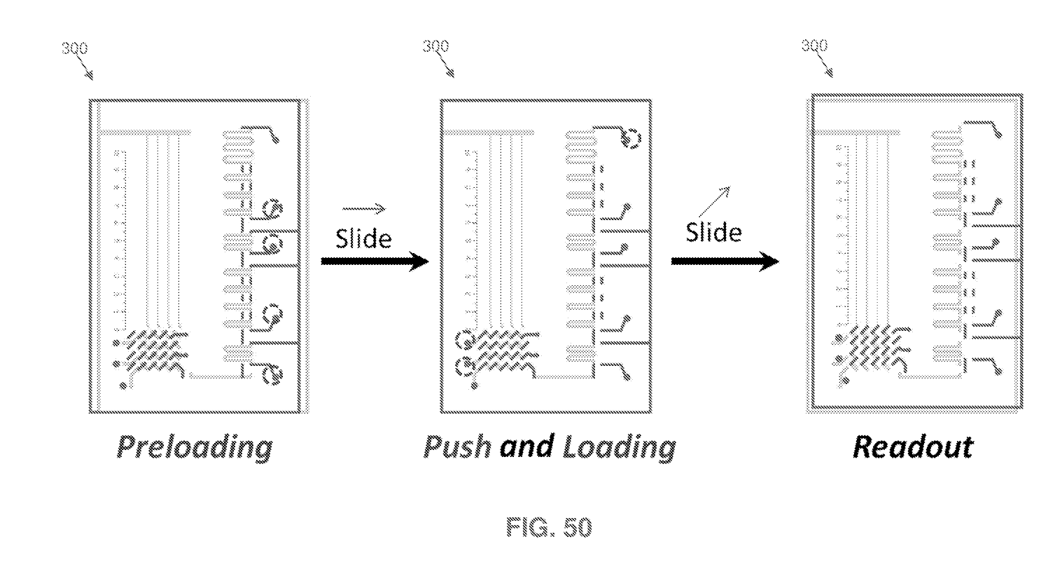

In still another embodiment of the present invention, and looking now at FIGS. 49 and 50, a novel multiplexed volumetric bar chart chip 300 is provided. Multiplexed volumetric bar chart chip 300 is similar to multiplexed volumetric bar chart chip 5 discussed above, except that multiplexed volumetric bar chart chip 300 is manufactured so as to reduce the reagent loading and washing steps required for a user.

In this embodiment, the ELISA reagents (i.e., the washing buffer, catalase probe and washing buffer) can be pre-loaded in the multiplexed volumetric bar chart chip during the manufacturing stage (e.g., at the locations shown in FIG. 49). At the time of use, the sample is positioned in the multiplexed volumetric bar chart chip (e.g., at the location shown in FIG. 49). Then, the multiplexed volumetric bar chart chip is slid vertically so that the sample, washing buffer, catalase probe and washing buffer are sequentially passed through the ELISA reagent row of the chip, whereby to prepare the ELISA row of the chip in a single action. Subsequently, the multiplexed volumetric bar chart chip can be slid in the oblique direction so as to activate the oxygen reaction and generate the desired results.

In this form of the invention, the user will only need to load the sample into the chip and then slide the chip obliquely so as to activate the assay process.

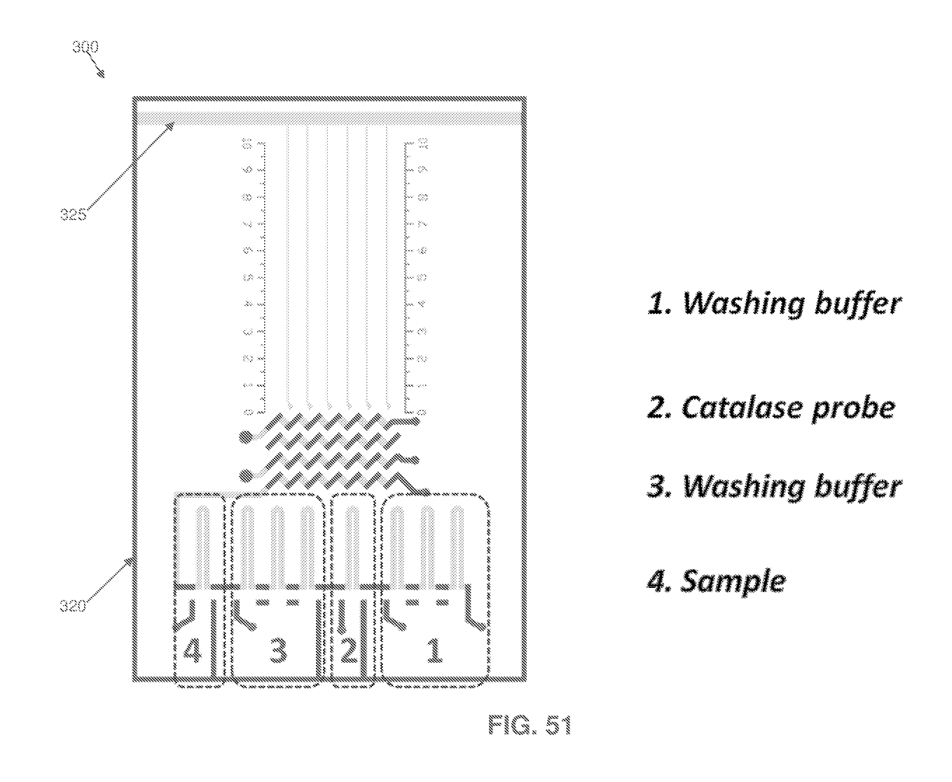

FIG. 51 shows another form of the present invention in which the multiplexed volumetric bar chart chip is configured to load the ELISA row of the chip through a horizontal motion.

### Platinum Nanoparticles

In the foregoing description, gas is generated by the reaction of an ELISA probe with a reagent, and specifically, gas is generated by the reaction of the ELISA probe (i.e., the protein-specific antibody which is bound to the target protein which is bound to the catalase) with hydrogen peroxide. It is important to note that many other combinations of a probe and a reagent may be used to generate gas. By way of example but not limitation, such probe and reagent combination may include catalase and hydrogen peroxide, platinum film or particles and hydrogen peroxide, catalase and carbamide peroxide, zinc and chloric acid, iron and chloric acid, and other similar combinations. Thus, since the multiplexed volumetric bar chart chip readout is based on the volumetric measurement of a gas generation, many fast responsive gas generation schemes can be used for the system, including catalase with hydrogen peroxide, platinum film or particles and hydrogen peroxide, catalase and carbamide peroxide, zinc and chloric acid, iron and chloric acid, and other similar combinations.

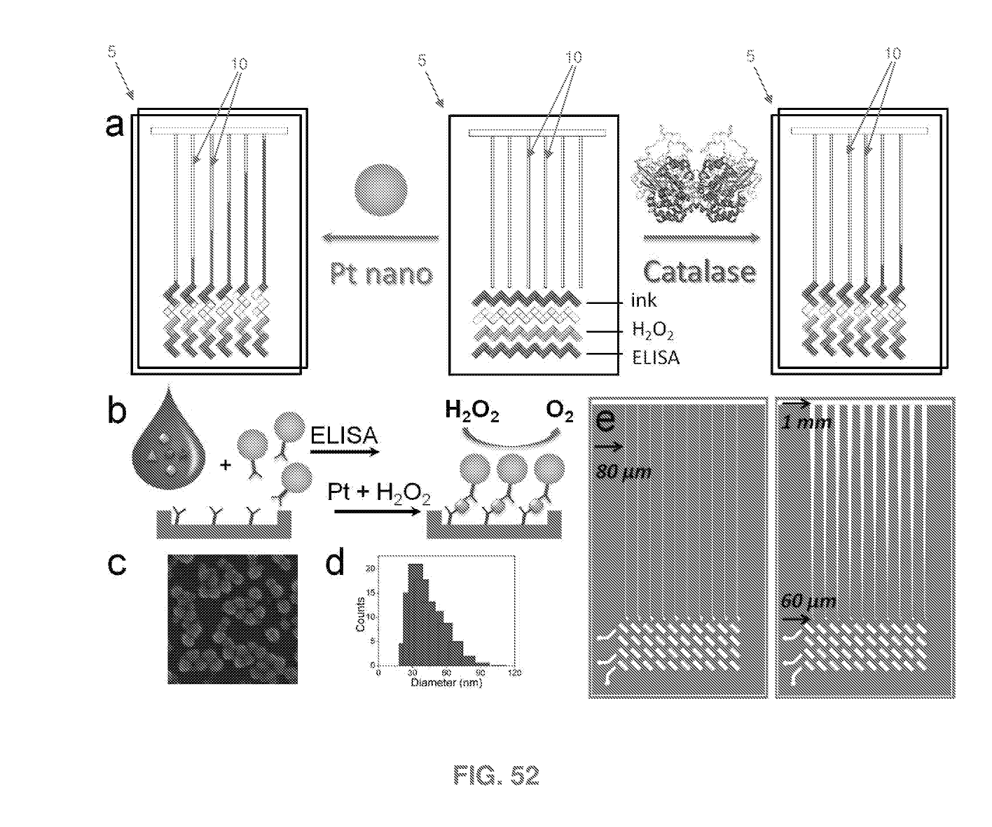

Thus, in another form of the present invention, platinum nanoparticles may be utilized in the place of catalase. In this form of the present invention, and looking now at FIG. 52 (as well as others of the figures), a sample (e.g., blood, urine, etc.) is introduced into inlet 50 of multiplexed volumetric bar chart chip 5 so that the sample fills the penultimate row 75B, causing the sample to mix with the different protein-specific antibodies which are bonded to bottom plate 20 in recesses 20, so that the target proteins bind to the appropriate protein-specific antibodies in the recesses 30. Row 75B is then flushed (e.g., with a buffer solution) so as to remove any materials which are not bound to a protein-specific antibody. Platinum nanoparticles conjugated with detection antibodies are then added into inlet 50 so as to fill penultimate row 75B. This action causes the platinum nanoparticles to bind to the target proteins which are themselves bound to the protein-specific antibodies in the recesses 30. The platinum nanoparticles are then rinsed from penultimate row 75B, leaving behind only those platinum nanoparticles which are bound to target proteins via their detection antibodies.

Thereafter, top plate 25 is slid obliquely relative to bottom plate 20, rows 75 are transformed into columns 100, and hydrogen peroxide contained in row 75A is permitted to advance up each of the columns 100 and thereby mix with any platinum nanoparticles bound to the target proteins, thereby permitting the reaction between the platinum nanoparticles and the hydrogen peroxide to produce oxygen gas, whereby to propel the red ink contained in columns 100 into and along bar columns 10.

Platinum nanoparticles exhibit several properties which can make them advantageous. By way of example but not limitation, catalase reacts with hydrogen peroxide for up to about 2 minutes, whereas platinum nanoparticles have no such limitation. Thus, in some situations, platinum nanoparticles can provide higher sensitivity and longer stability than catalase.

### Serpentine Channels

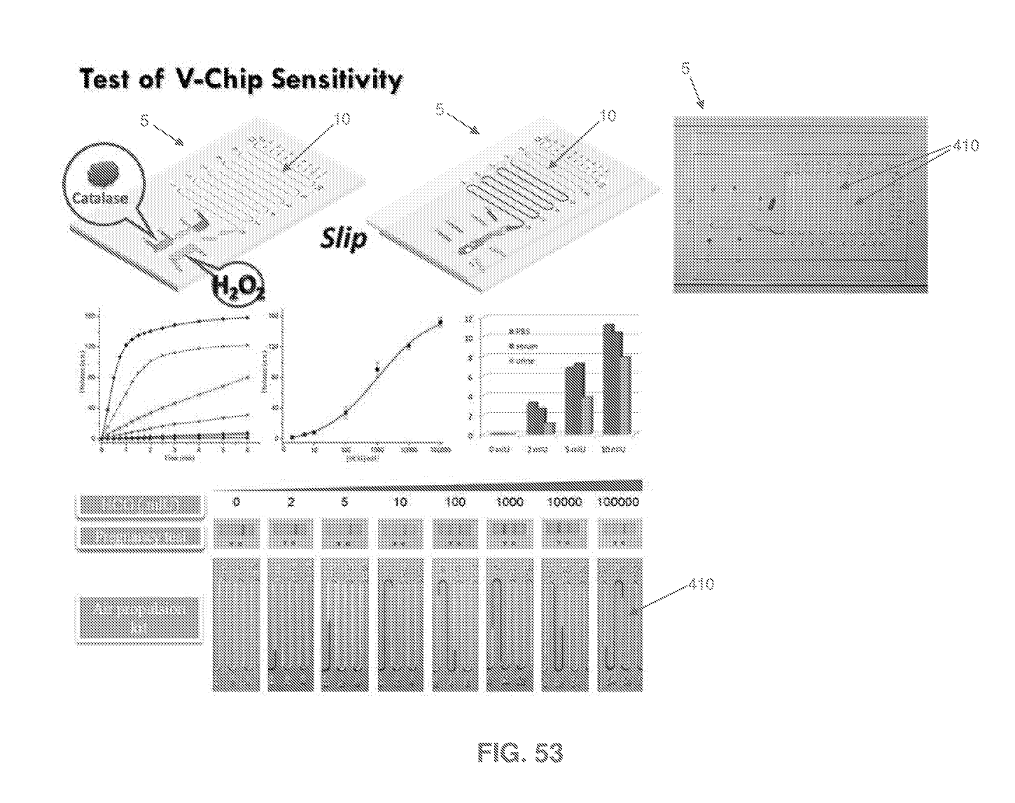

In the foregoing description, bar channels 10 are generally discussed in the context of a bar chart, where a plurality of straight bar channels 10 are arranged in parallel so as to provide a series of discrete channels. However, as also noted above, bar channels 10 may be curved (e.g., serpentine, circular, z-shaped) or formed in other configuration which provides a series of channels having a length. In addition, it should also be appreciated that, if desired, bar channels 10 may comprise a single serpentine pathway 410 (see FIG. 53). Providing a single serpentine pathway may be advantageous in situations where the target protein is present in a high concentration and quantitation is desired (e.g., as may be desired in a pregnancy test).

### Channel Variations

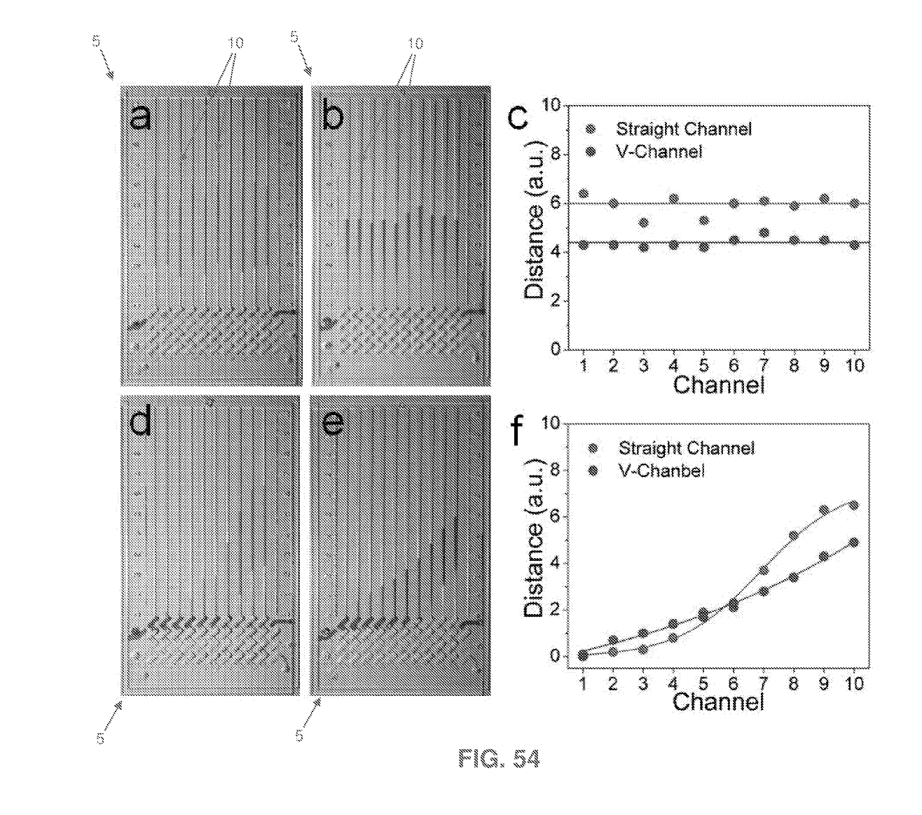

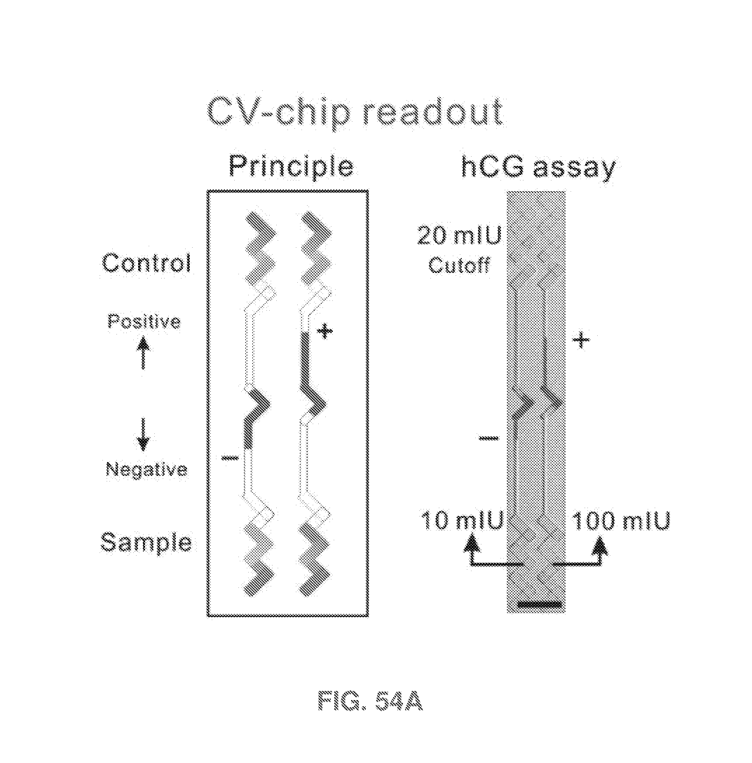

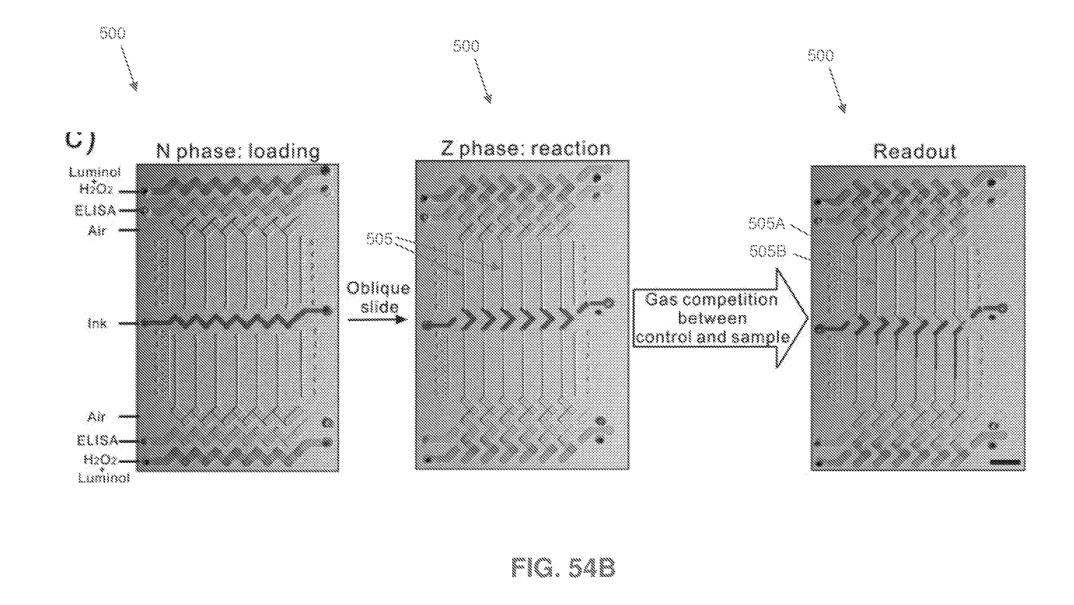

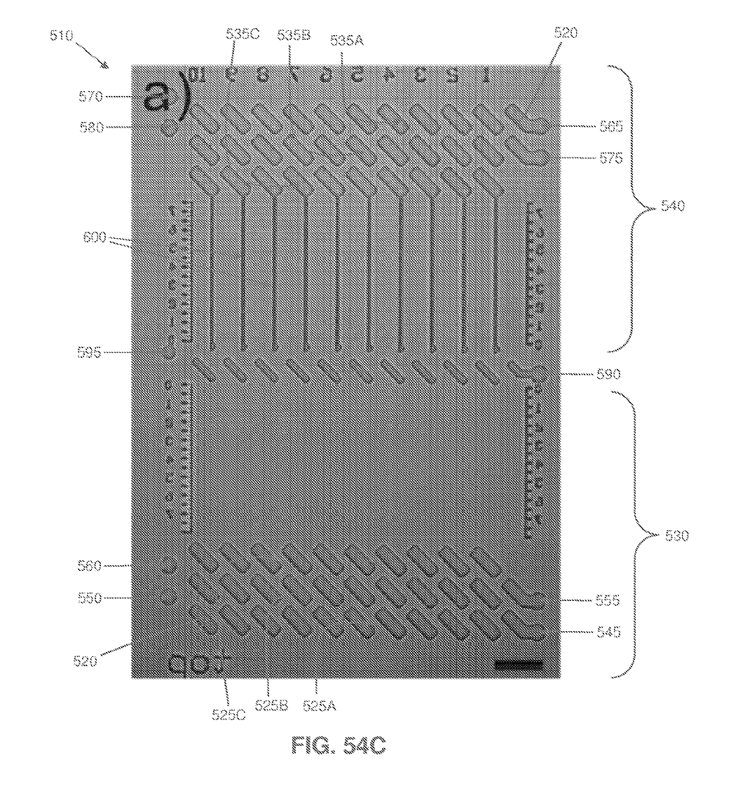

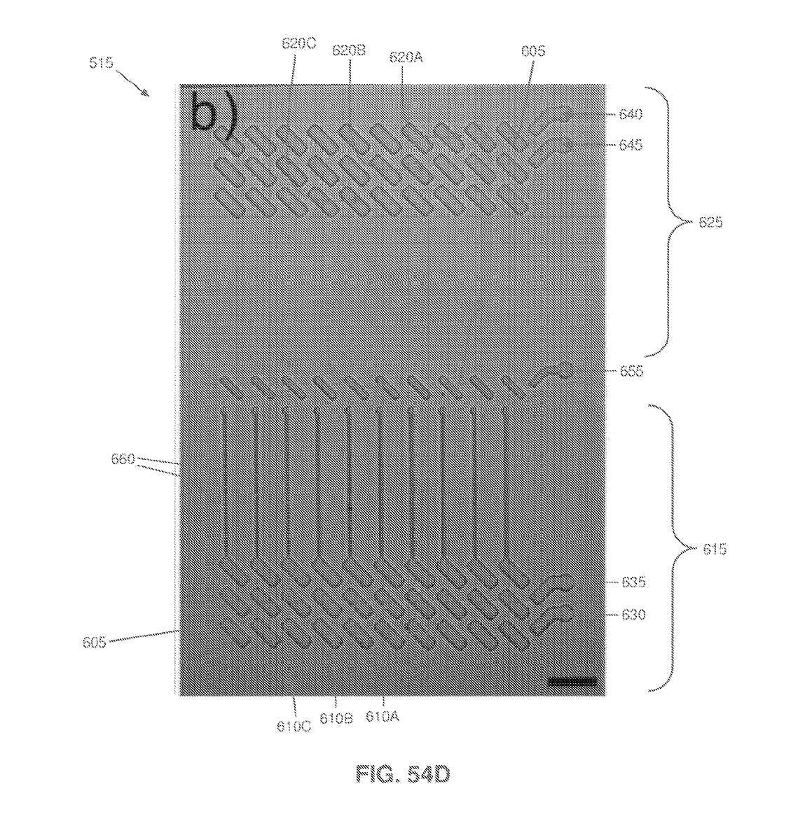

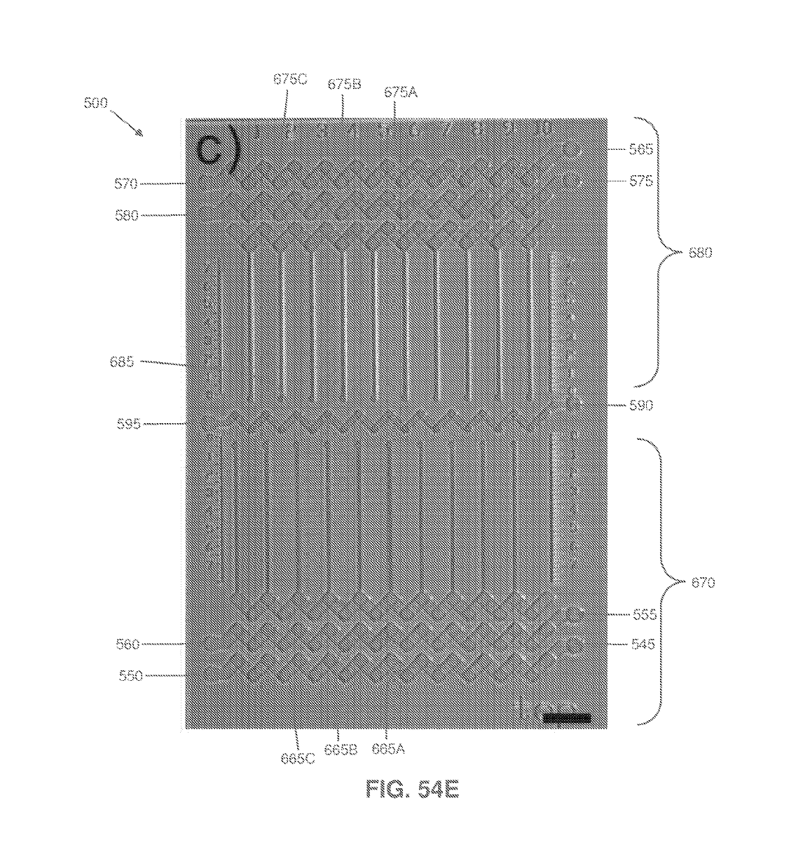

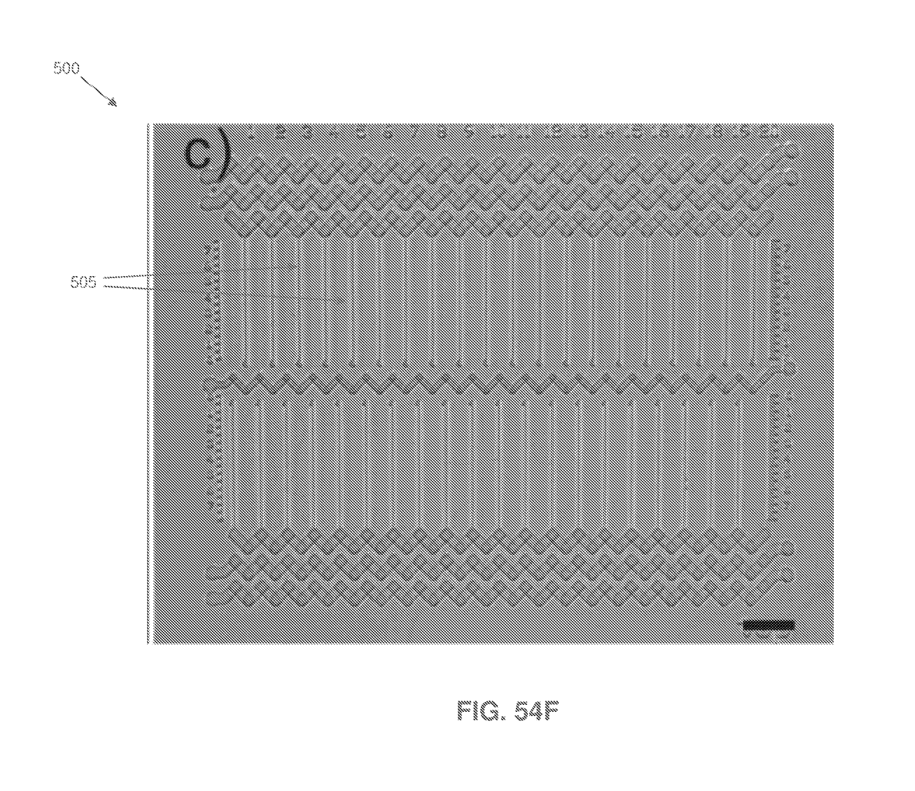

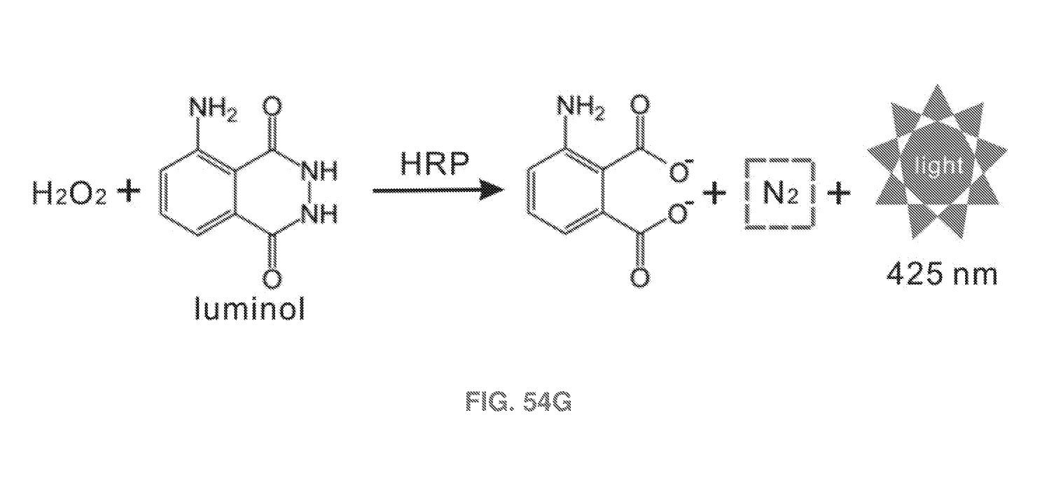

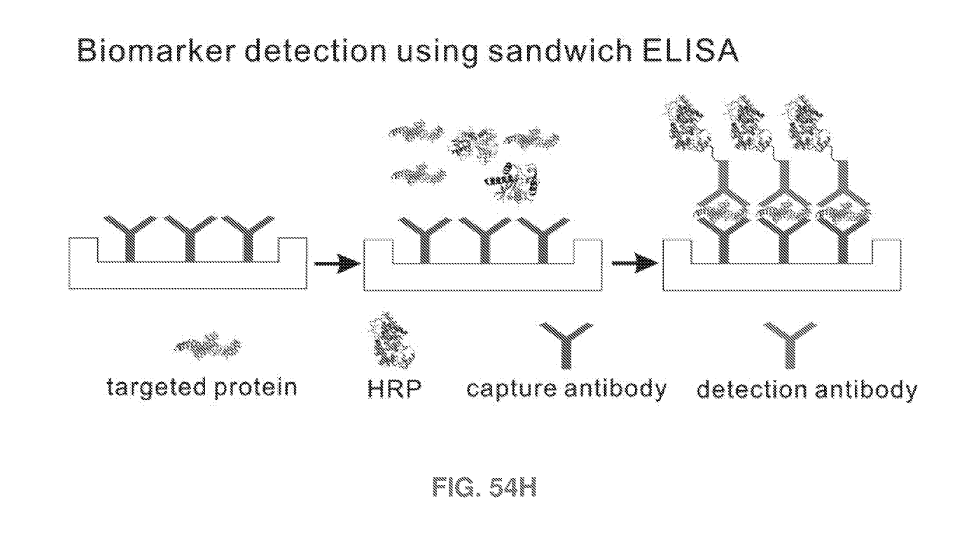

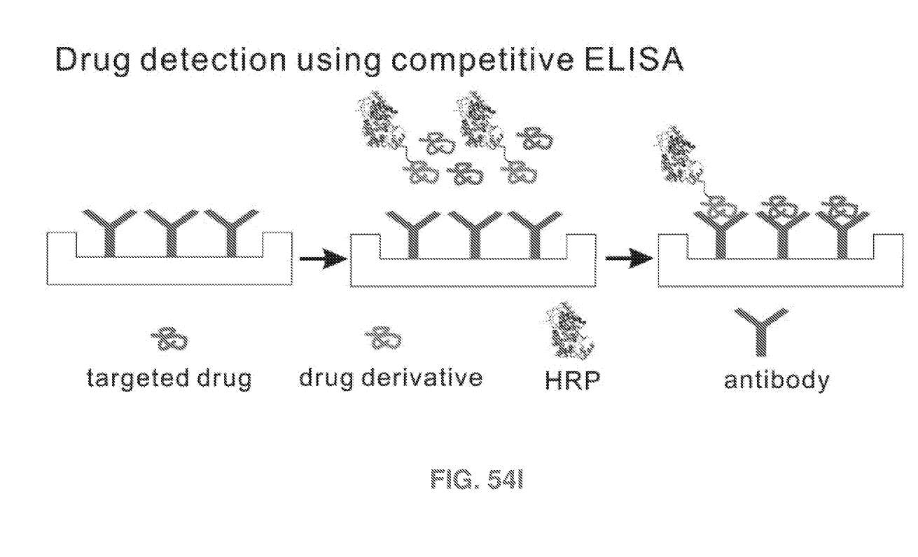

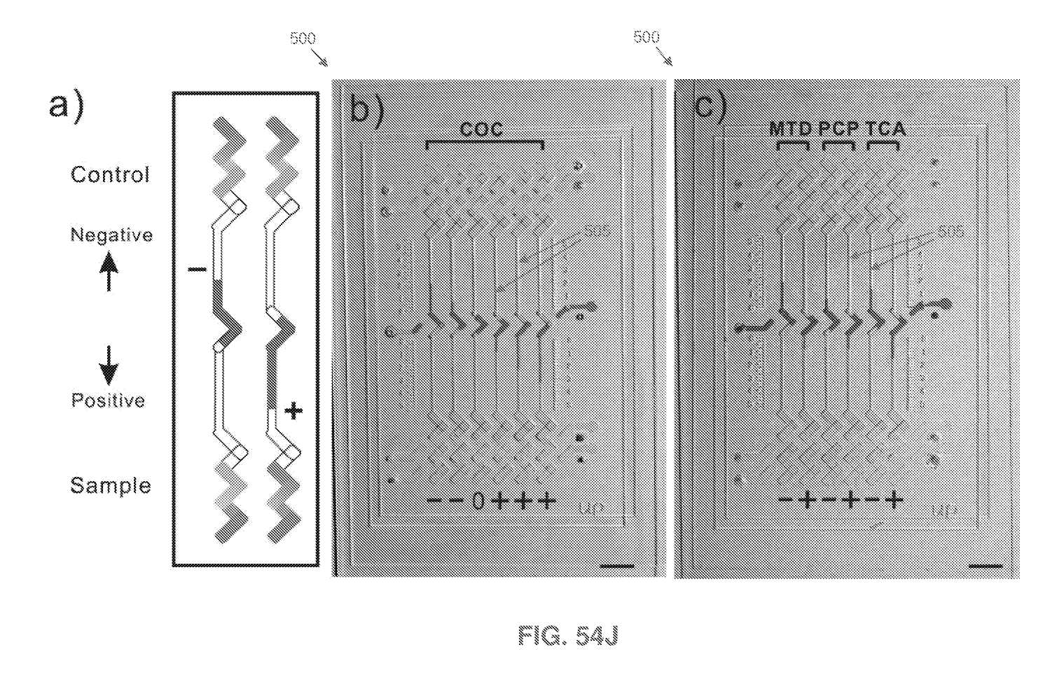

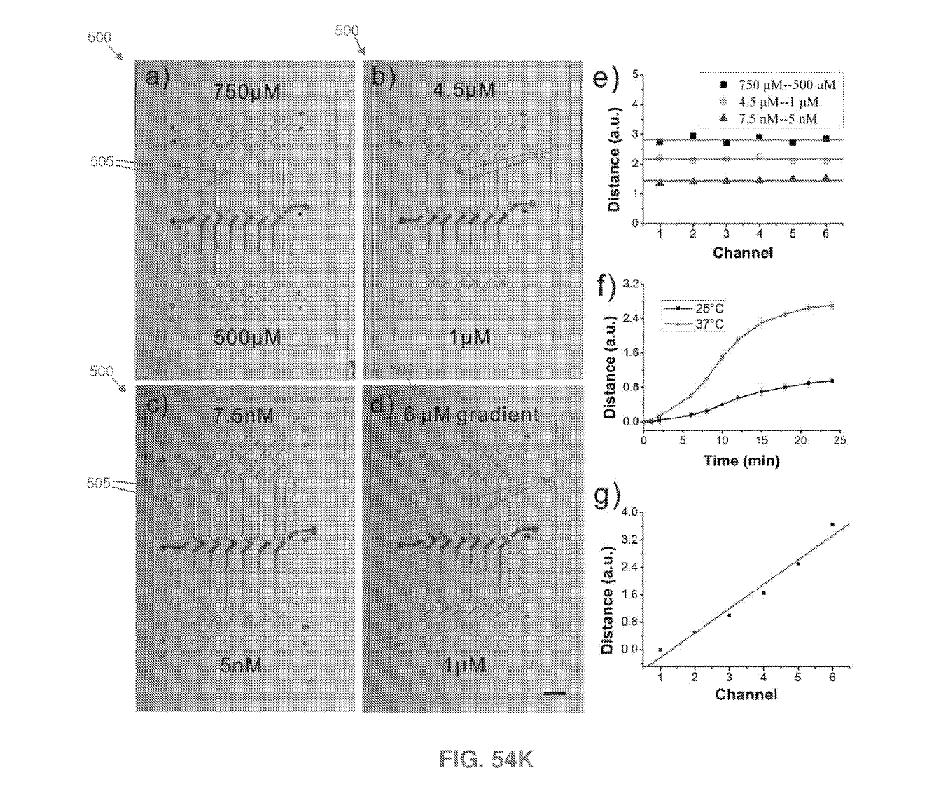

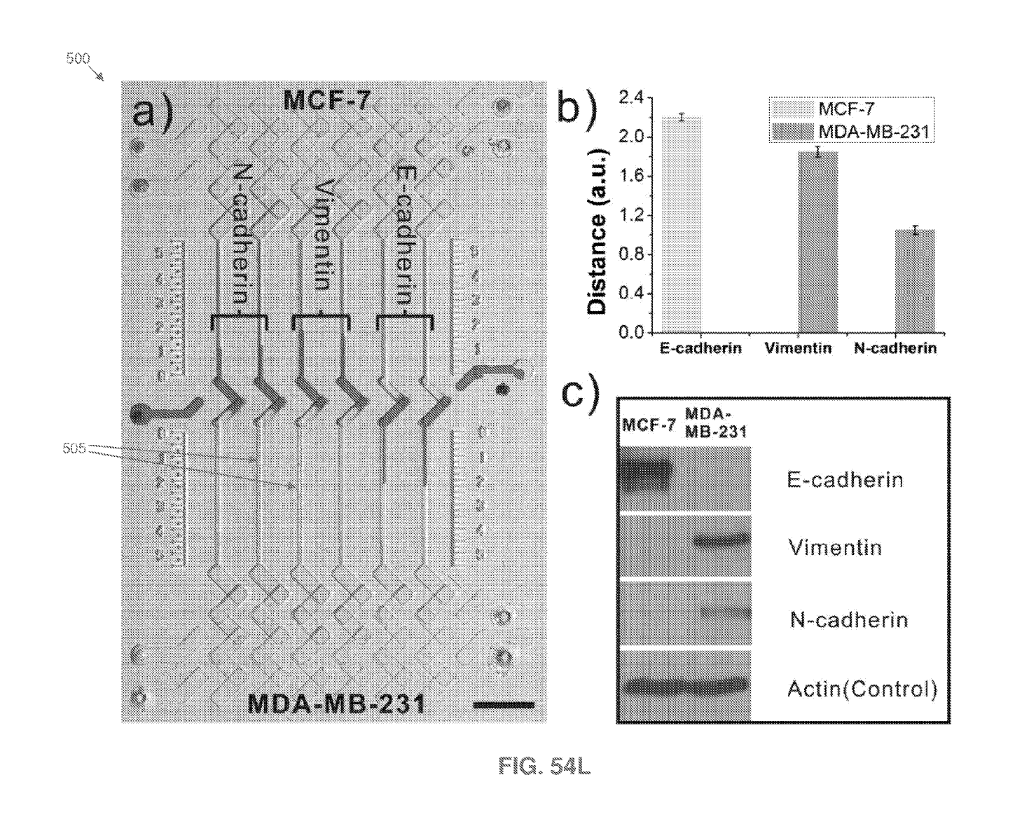

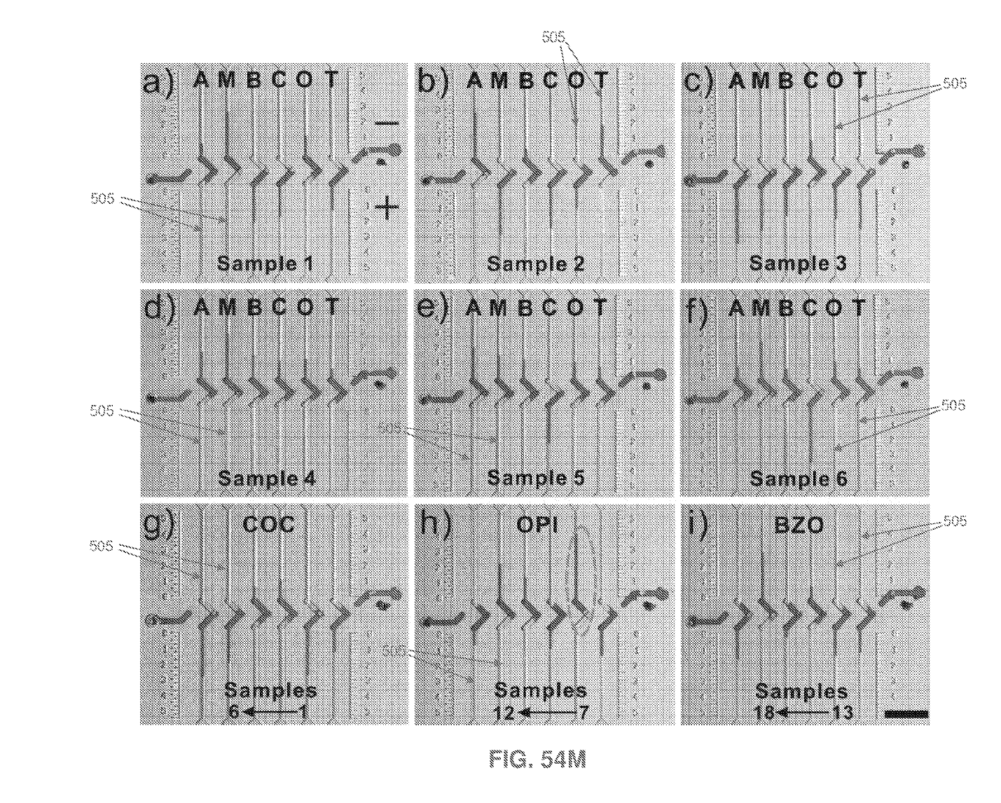

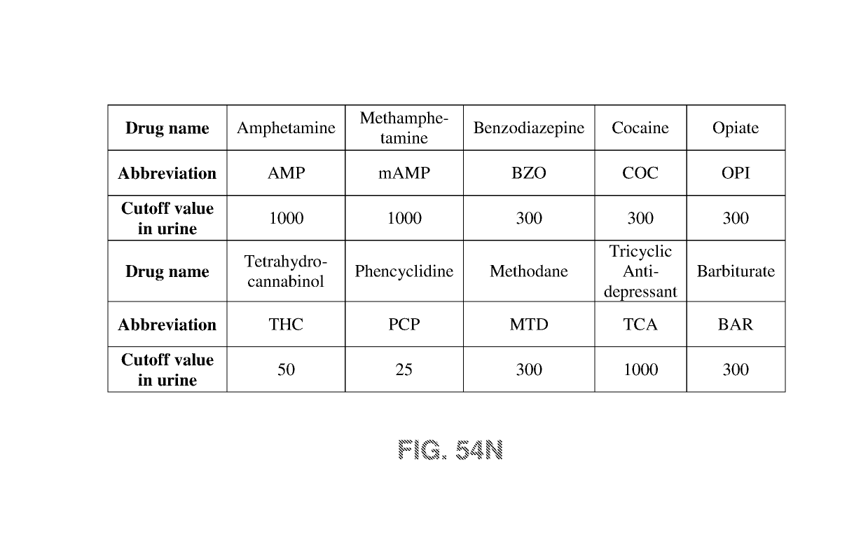

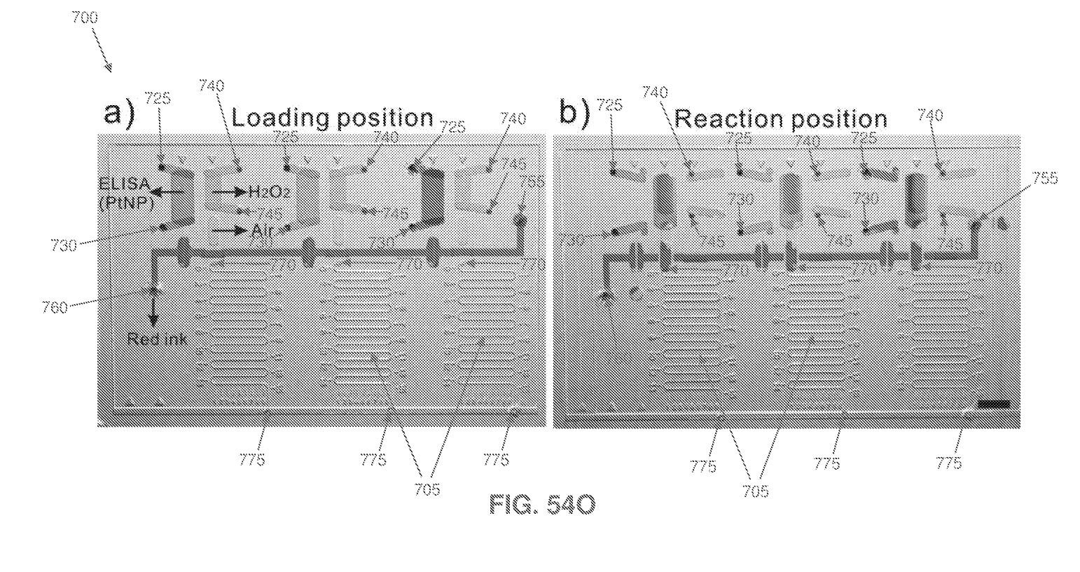

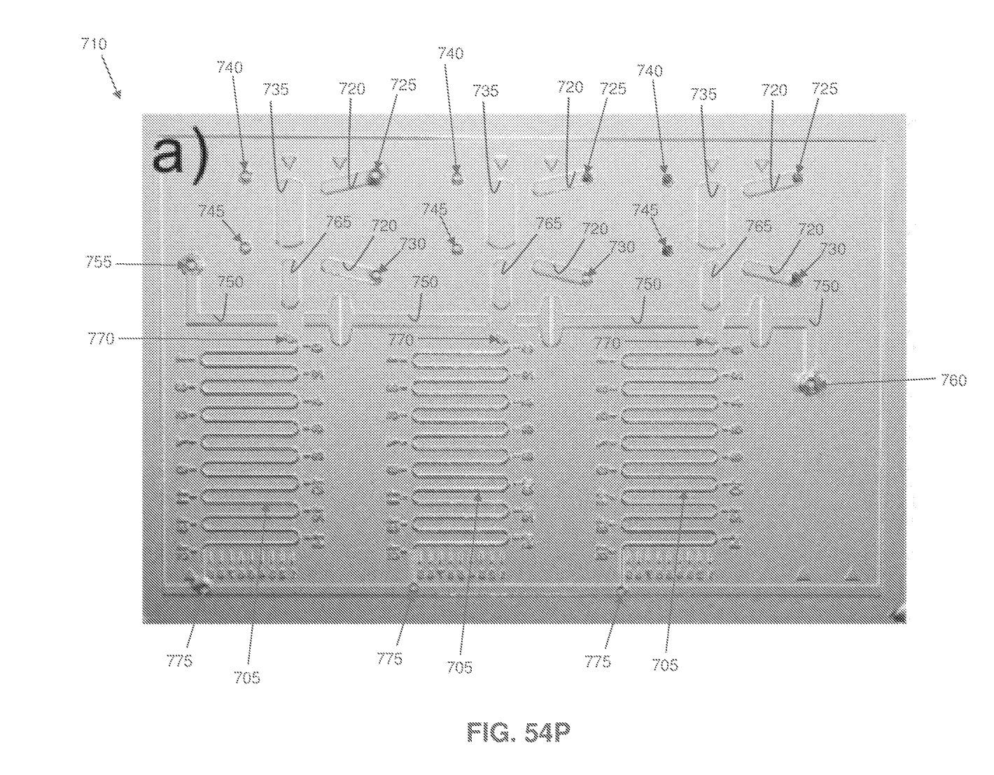

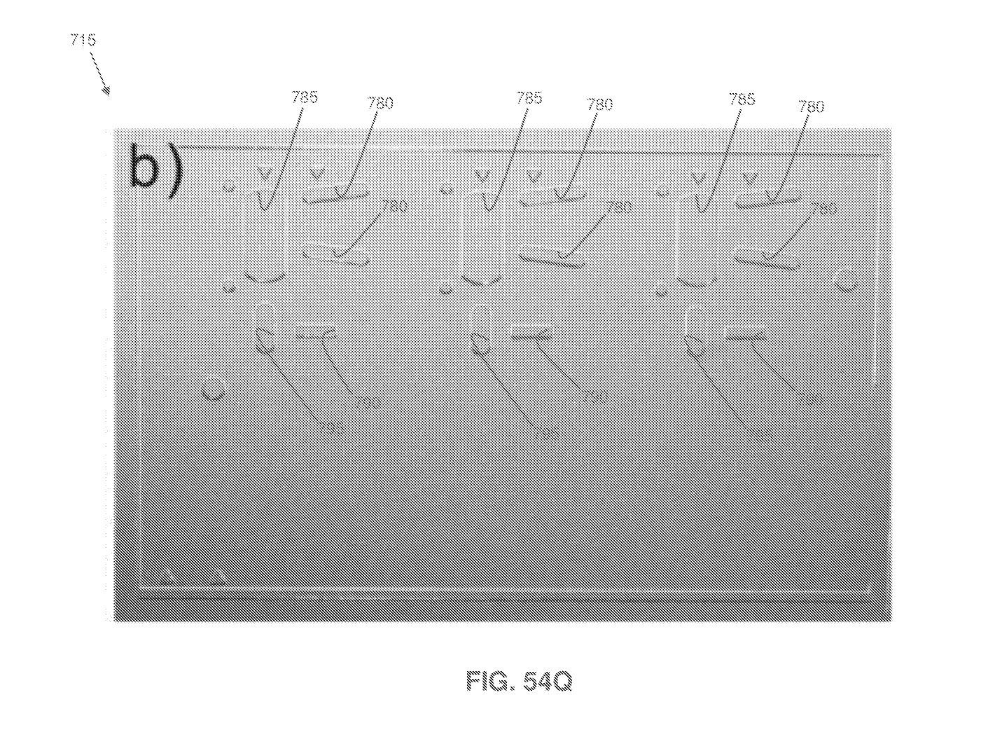

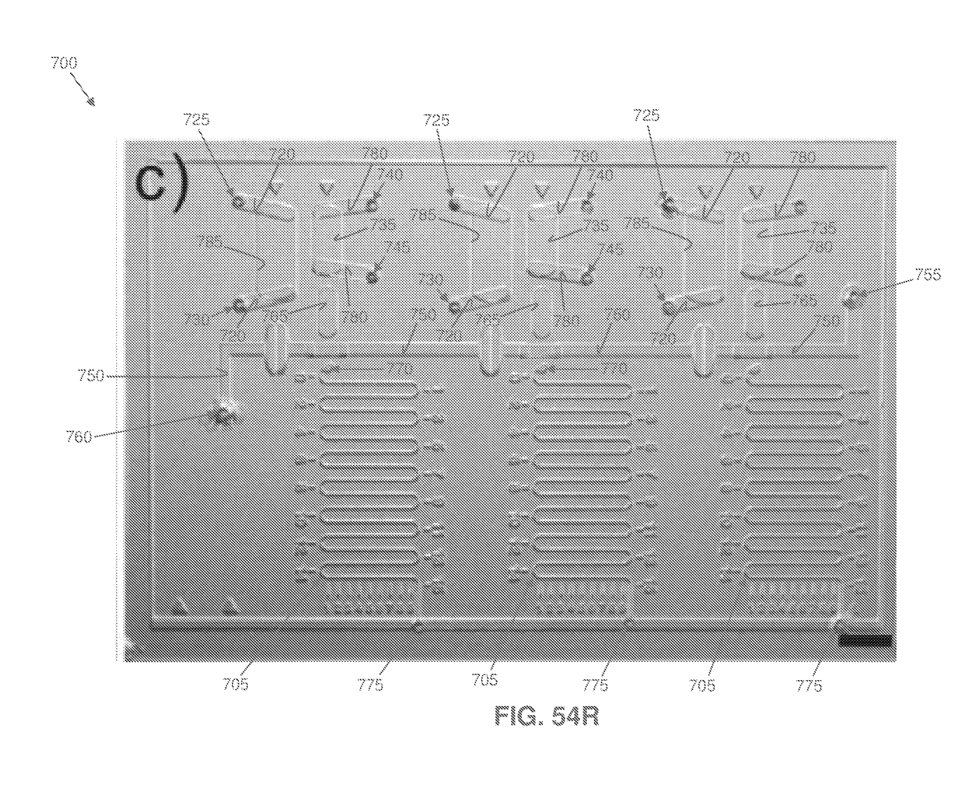

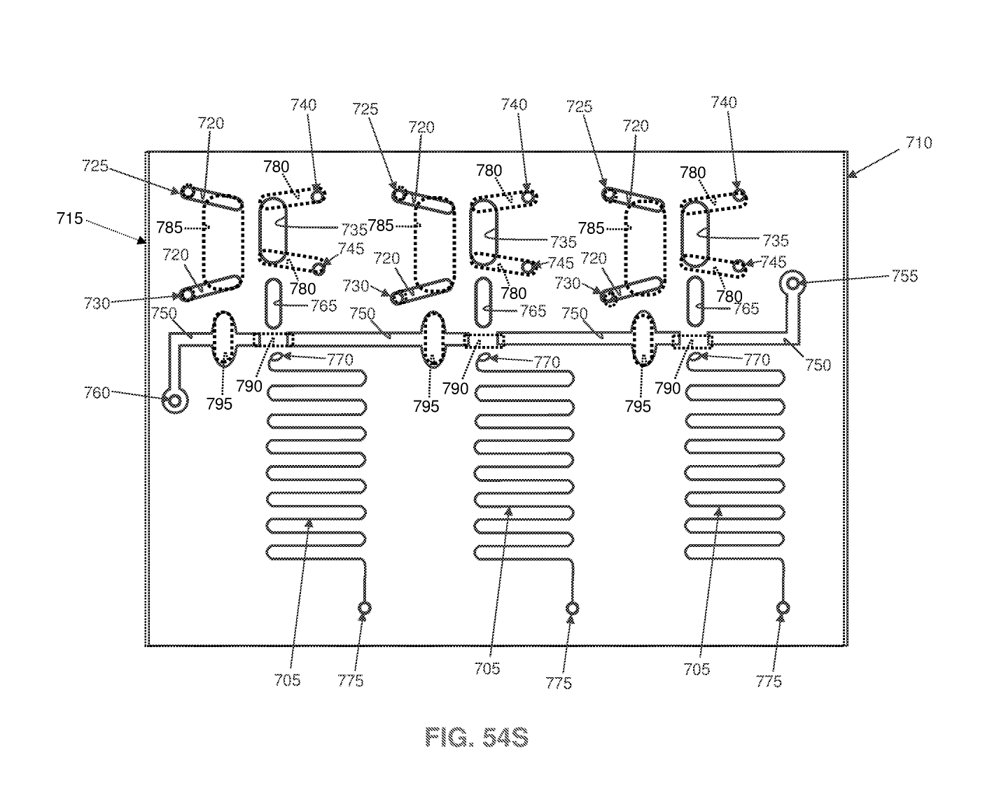

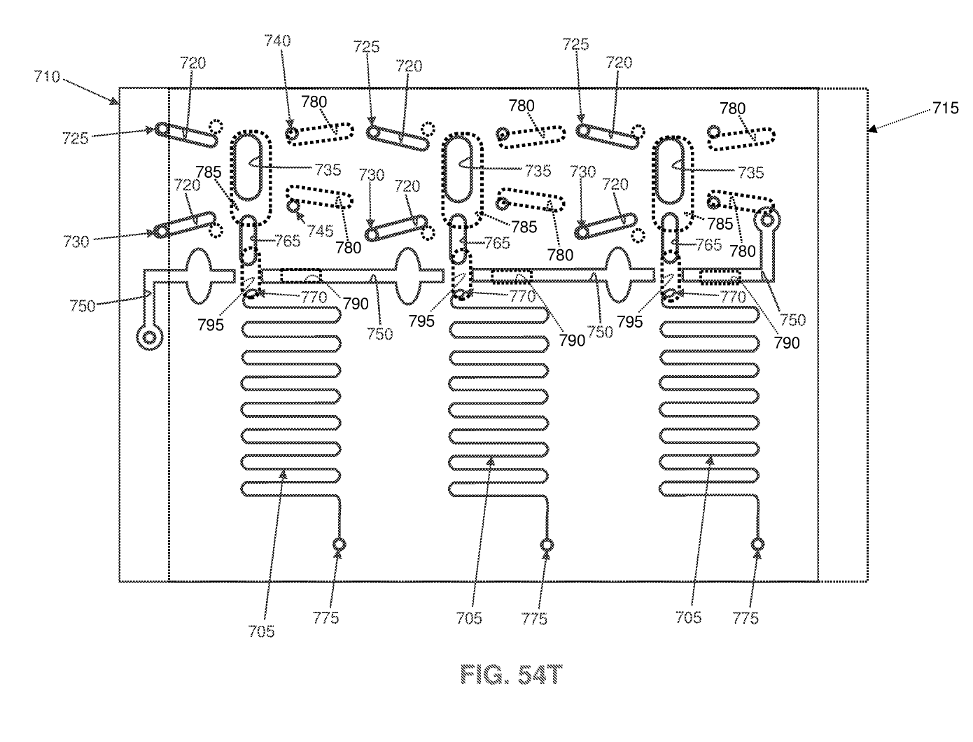

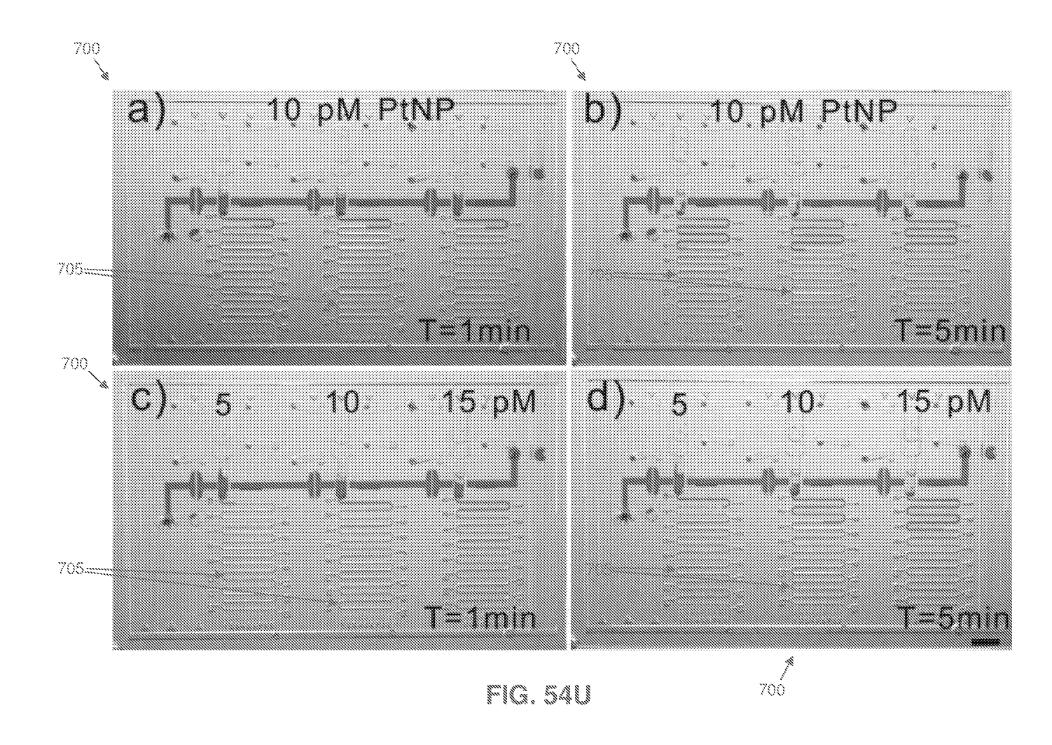

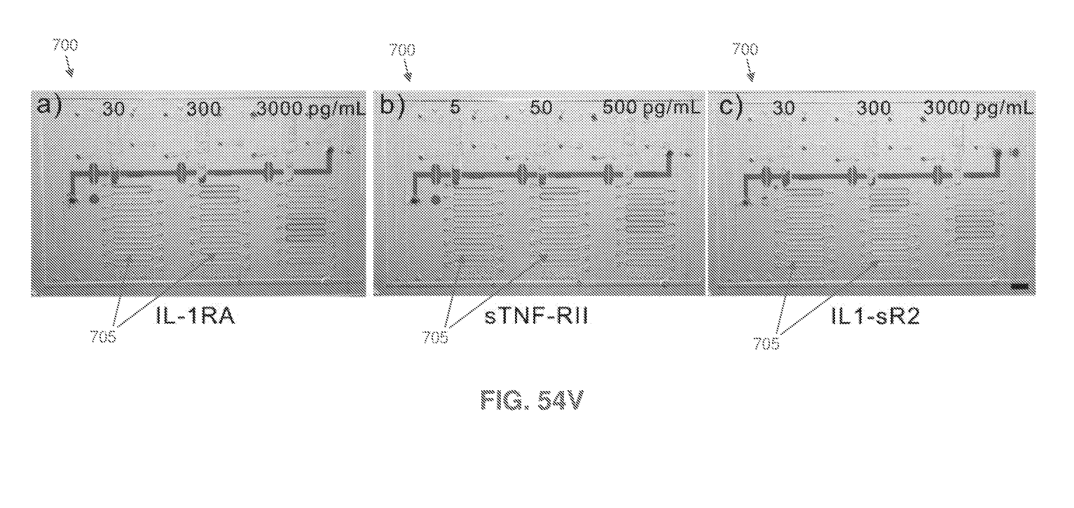

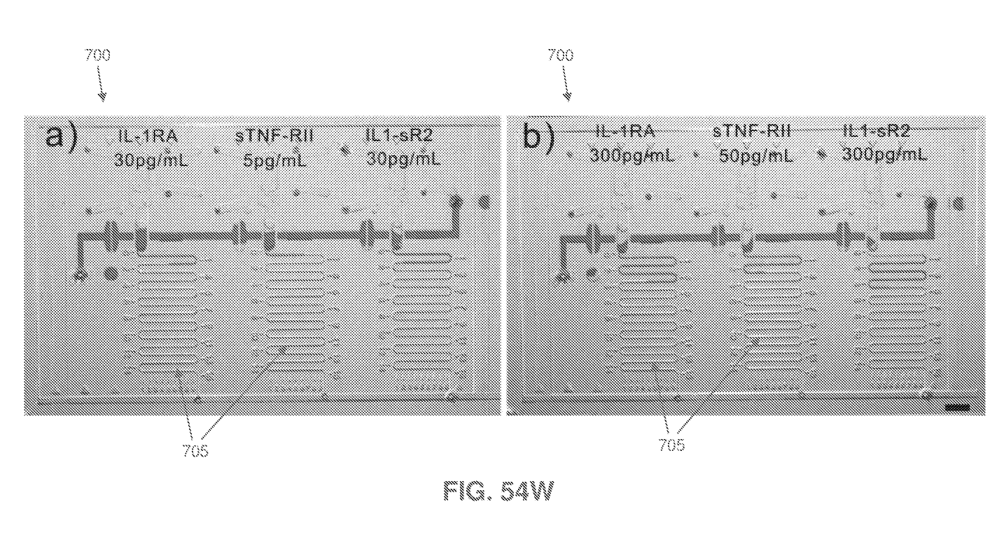

If desired, the width and/or depth of channels 10 may vary along the length of the channels. By way of example but not limitation, and looking now at FIG. 54, channels 10 may comprise a V-shape, where the distal end (i.e., the terminal portion) of channel 10 is of greater width than the width of the channel at its proximal end. Additionally and/or alternatively, the depth of channel 10 may also be varied along the length of the channel (e.g., to provide a deeper channel 10 toward the distal end of the channel). By varying the width and/or depth of channel 10 along its length, the volume of the interior of the channel may be varied, whereby to provide additional time/space for the advancement of the red ink during a reaction. By way of example but not limitation, such a construction may be advantageous to obtain a higher sensitivity and a larger dynamic range for a desired assay.

### Multiplexed Volumetric Bar Chart Chip Utilizing “Competitive” Control

In another embodiment of the present invention, and looking now at FIG. 54A, a novel multiplexed volumetric bar chart chip is provided which may be used in accordance with the present invention to determine whether a threshold quantity of a target protein (or other types of biomarkers or other analytes) is present in a sample. With this form of the invention, a reaction with a control is used to generate a gas that acts in direct competition with a gas that is generated by a reaction with a sample, whereby to provide a multiplexed volumetric bar chart chip which exhibits a clear positive or negative indication of the presence of an analyte (i.e., the ink moves from a central location in a column on the multiplexed volumetric bar chart chip into either the “positive” side of the multiplexed volumetric bar chart chip or the “negative” side of the multiplexed volumetric bar chart chip). This form of the invention also allows for the setting of a predetermined threshold value for detecting an analyte, i.e., the concentration of the control can be selected so as to reduce false positives when the concentration of the analyte is extremely low. Also, this form of the invention minimizes the influence of environmental conditions (e.g., temperature, humidity, pH, ionic strength, etc.) on the assay, and eliminates the need for calibration of the multiplexed volumetric bar chart chip, by placing the control and the sample in direct “competition” with one another. Put another way, since the control and the sample are subject to the same environmental conditions, the effects of those environmental conditions are effectively canceled out.

More particularly, and looking now at FIG. 54B, in this form of the invention, there is provided a novel multiplexed volumetric bar chart chip 500. Multiplexed volumetric bar chart chip 500 is configured to simultaneously determine (i) whether an analyte is present in a sample at a concentration above a predetermined threshold value, and (ii) the quantity of multiple analytes which may be present in a sample, with the quantity of each analyte which is present in the sample being indicated in a particular one of a plurality of bar channels 505. By way of example but not limitation, 6, 10, 20, 30, and 50-plexed, or more than 50-plexed, bar channels may be incorporated into multiplexed volumetric bar chart chip 500 (see, for example, FIG. 54F which shows a 20-plexed volumetric bar chart chip 500). Bar channels 505 may be straight or curved (e.g., serpentine, circular, z-shaped) or formed in any other configuration which provides a series of channels having a length, wherein each of the series of channels preferably have the same configuration and orientation. As a result of this construction, the review of a particular bar channel 505 will indicate (i) whether an analyte is present in a sample at a concentration above a predetermined threshold value, and (ii) the quantity of a particular analyte which may be present in the sample and, significantly, the collective array of the plurality of bar channels 505 will simultaneously indicate, in bar chart form, the presence of and quantities of multiple analytes which may be present in the sample, whereby to provide multi-analyte quantity measurements and hence a more comprehensive diagnostic result.

Looking now at FIGS. 54C, 54D and 54E, multiplexed volumetric bar chart chip 500 comprises two plates, a transparent top plate 510 and a bottom plate 515 (which may or may not be transparent).

More particularly, top plate 510 (FIG. 54C) has a plurality of recesses 520 formed on its bottom surface, with recesses 520 being arranged in a plurality of sample rows 525 (i.e., 525A, 525B, and 525C) arrayed along the lower (i.e., “sample”) portion 530 of the bottom surface of top plate 510, and a plurality of control rows 535 (i.e., 535A, 535B, and 535C) arrayed along the upper (i.e., “control”) portion 540 of the bottom surface of top plate 510. Each of the recesses 520 extends at a 45 degree angle relative to the axis of a given sample row 525 or control row 535, with each recess 520 in one row 525, 535 being aligned with an offset recess 520 in an adjacent row 525, 535.

An inlet 545 is connected to a far side recess 520 on the ultimate sample row 525A, and an outlet 550 is formed adjacent to the opposite far side recess 520 on the same ultimate sample row 525A. An inlet 555 is connected to a far side recess 520 on the penultimate sample row 525B, and an outlet 560 is formed adjacent to the opposite far side recess 520 on the same penultimate sample row 525B. The antepenultimate sample row 525C lacks both an inlet and an outlet.

An inlet 565 is connected to a far side recess 520 on the ultimate control row 535A, and an outlet 570 is formed adjacent to the opposite far side recess 520 on the same ultimate control row 535A. An inlet 575 is connected to a far side recess 520 on the penultimate control row 535B, and an outlet 580 is formed adjacent to the opposite far side recess 520 on the same penultimate control row 535B. The antepenultimate control row 535C lacks both an inlet and an outlet.

A plurality of recesses 520 are also formed on the bottom surface of top plate 510 intermediate sample portion 530 and control portion 540, whereby to form an ink row 585. An inlet 590 is connected to a far side recess 520 on ink row 585, and an outlet 595 is formed adjacent to the opposite far side recess 520 on ink row 585.

In addition, a plurality of bar channels 600 are formed on the bottom surface of top plate 510, with each of the bar channels 600 being connected to a recess 520 in the antepenultimate row 535C of control portion 540, and with each of the bar channels 600 extending from antepenultimate row 535C toward ink row 585, parallel to one another and perpendicular to the axis of rows 525, 535.

In one preferred form of the invention, recesses 520, inlets 545, 555, 565, 575, 590, outlets 550, 560, 570, 580, 595, and bar channels 600 are all formed in the bottom surface of top plate 510 using a conventional etching process of the sort well known in the etching arts. Preferably, recesses 520, inlets 545, 555, 565, 575, 590, outlets 550, 560, 570, 580, 595 and bar channels 600 are etched in the bottom surface of a glass plate. Alternatively, recesses 520, inlets 545, 555, 565, 575, 590, outlets 550, 560, 570, 580, 595 and bar channels 600 may be formed in a silicon plate, a plastic plate, a ceramic plate, a quartz plate, a metal oxide plate or other appropriate substrate material.

Bottom plate 515 (FIG. 54D) has a plurality of recesses 605 formed on its top surface, with recesses 605 being arranged in a plurality of sample rows 610 (i.e., 610A, 610B and 610C) arrayed along the lower (i.e., “sample”) portion 615 of the top surface of bottom plate 515, and a plurality of control rows 620 (i.e., 620A, 620B, and 620C) arrayed along the upper (i.e., “control”) portion 625 of the top surface of bottom plate 515. Each of the recesses 605 extends at a 45 degree angle relative to the axis of a given sample row 610 or control row 620, with each recess 605 in one row 610, 620 being aligned with an offset recess 605 in an adjacent row 610, 620.

An outlet 630 is connected to a far side recess 605 on the ultimate sample row 610A. An outlet 635 is connected to a far side recess 605 on the penultimate sample row 610B. The antepenultimate sample row 610C lacks both an inlet and an outlet. An outlet 640 is connected to a far side recess 605 on the ultimate control row 620A. An outlet 645 is connected to a far side recess 605 on the penultimate control row 620B. The antepenultimate control row 620C lacks an both an inlet and an outlet.

A plurality of recesses 605 are also formed on the top surface of bottom plate 515 intermediate sample portion 615 and control portion 625, whereby to form an ink row 650. An outlet 655 is connected to a far side recess 605 on the ink row 650.

In addition, a plurality of bar channels 660 are formed on the top surface of bottom plate 515, with each of the bar channels 660 being connected to a recess 605 in the antepenultimate row 610C of sample portion 615, and with each of the bar channels 660 extending from antepenultimate row 610C toward ink row 650, parallel to one another and perpendicular to the axis of rows 610, 620.

In one preferred form of the invention, recesses 605, outlets 630, 635, 640, 645, 655 and bar channels 660 are all formed in the top surface of bottom plate 515 using a conventional etching process of the sort well known in the etching arts. Preferably, recesses 605, outlets 630, 635, 640, 645, 655 and bar channels 660 are etched in the top surface of a glass plate. Alternatively, recesses 605, outlets 630, 635, 640, 645, 655 and bar channels 660 may be formed in a silicon plate, a plastic plate, a ceramic plate, a quartz plate, a metal oxide plate or other appropriate substrate material.

### Assembly of Multiplexed Volumetric Bar Chart Chip 500

Looking next at FIG. 54E, top plate 510 is assembled on top of bottom plate 515 so that recesses 520 in top plate 510 communicate with recesses 605 in bottom plate 515. More particularly, when top plate 510 is assembled on top of bottom plate 515 in this manner, recesses 520 in top plate 510 will cooperate with recesses 605 in bottom plate 515 so as to initially form a plurality of continuous sample rows 665 (i.e., 665A, 665B, and 665C) arrayed along the lower (i.e., “sample”) portion 670 of multiplexed volumetric bar chart chip 500, a plurality of continuous control rows 675 (i.e., 675A, 675B and 675C) arrayed along the upper (i.e., “control”) portion 680 of multiplexed volumetric bar chart chip 500, and a continuous ink row 685 in multiplexed volumetric bar chart chip 500. By virtue of this construction, inlet 545 of ultimate sample row 665A is connected with the outlet 550 of ultimate sample row 665A, inlet 555 of the penultimate sample row 665B is connected with the outlet 560 of penultimate sample row 665B, inlet 565 of ultimate control row 675A is connected with outlet 570 of ultimate control row 675A, inlet 575 of penultimate control row 675B is connected with outlet 580 of penultimate control row 675B, and inlet 590 of ink row 685 is connected with outlet 595 of ink row 685. Antepenultimate sample row 665C and antepenultimate control row 675C lack both an inlet and an outlet.

It will be appreciated that, due to the dispositions of recesses 520 in top plate 510 and recesses 605 in bottom plate 515, an oblique slide of top plate 510 relative to bottom plate 515 disrupts the aforementioned rows 665, 675, 685 and causes them to transform into a plurality of continuous columns (i.e., bar channels 505), with each bar channel 505 being in fluid communication with the aforementioned bar channels 600, 660. See FIG. 54B.

### Determining the Quantity of Multiple Analytes Present in a Sample Using the Multiplexed Volumetric Bar Chart Chip 500 while Utilizing a “Competitive” Control

In view of the foregoing construction, multiplexed volumetric bar chart chip 500 can be used to simultaneously determine whether a predetermined threshold quantity of a target protein or other types of biomarkers or other analytes is present in a sample, and the quantity of multiple analytes present in a sample, with the quantity of each specific analyte being indicated in a particular one of the plurality of bar channels 505.

More particularly, and referring now to FIGS. 54G, 54H and 54I, and as will hereinafter be discussed in further detail below, during manufacture of multiplexed volumetric bar chart chip 500, an analyte-specific antibody is bonded in a recess 520 of the penultimate sample row 525B of top plate 510 (and/or, if desired, in a recess 605 of the penultimate sample row 610B of bottom plate 515). If desired, an analyte-specific antibody may also be bonded in a recess 520 of the penultimate control row 535B of top plate 510 (and/or, if desired, in a recess 605 of the penultimate control row 620B of bottom plate 515).

In a preferred form of the present invention, a different analyte-specific antibody is bonded in each recess 520 of top plate 510.

As a result, after bottom plate 515 and top plate 510 are assembled together, continuous penultimate sample row 665B will contain a series of different analyte-specific antibodies, with a different analyte-specific antibody being located in each recess 520 of the continuous penultimate sample row 665B of top plate 510.

Prior to use, hydrogen peroxide (H₂O₂) is introduced into inlet 545 (FIG. 54E) of multiplexed volumetric bar chart chip 500, whereby to fill the continuous ultimate sample row 665A of multiplexed volumetric bar chart chip 500 with hydrogen peroxide. Hydrogen peroxide is also introduced into inlet 565 of multiplexed volumetric bar chart chip 500, whereby to fill the continuous ultimate control row 675A of multiplexed volumetric bar chart chip 500 with hydrogen peroxide. If desired, luminol may be added to the hydrogen peroxide before the hydrogen peroxide is introduced into inlets 545, 565. Red ink (or some other colored material which is readily discernible through top plate 510 and against bottom plate 515) is introduced into inlet 590 of multiplexed volumetric bar chart chip 500, whereby to fill continuous ink row 685 of multiplexed volumetric bar chart chip 500 with red ink. Continuous antepenultimate sample row 665C and continuous antepenultimate control row 675C are intentionally left empty to serve as an air spacer.

It should be appreciated that the foregoing construction may be utilized with different types of assays and/or with different probes and/or reagents. As will hereinafter be discussed in greater detail, two preferred types of assays are the “sandwich” ELISA method and the “competitive” ELISA method. Both types of assays rely on a chemical reaction which generates a gas that moves the ink in continuous ink row 685 into bar channels 660 and/or bar channels 600.

1. The “Sandwich” ELISA Assay

When the “sandwich” ELISA method is used for the assay, the target analyte (i.e., the analyte being tested for) will bind the analyte-specific antibody which is bound to a recess 520 of continuous penultimate sample row 665B, a probe (e.g., an antibody-enzyme conjugate) will then bind the analyte, and a chemical reaction will be used to generate a gas, as will hereinafter be discussed in greater detail.

First, the sample is introduced into inlet 555 (FIG. 54E) of multiplexed volumetric bar chart chip 500, so that the sample fills continuous penultimate sample row 665B. After a period of time, the continuous penultimate sample row 665B is washed, leaving the target analyte(s) (where present) bound to the analyte-specific antibodies which are, in turn, bound to a recess 520 of continuous penultimate sample row 665B. At this point, the amount of the target analyte retained in continuous penultimate sample row 665B is proportional to the amount of the target analyte present in the sample.

A conjugate comprising horseradish peroxidase (HRP) bound to a detection antibody (which detection antibody is selected because it will bind the target analyte) is prepared. The HRP-detection antibody conjugate is introduced into inlet 555 of multiplexed bar chart chip 500, so that the HRP-detection antibody conjugate fills continuous penultimate sample row 665B. Where the target analyte is present (i.e., where the target analyte is bound to an analyte-specific antibody bound to recess 520), the HRP-detection antibody conjugate will bind the target analyte. Continuous penultimate sample row 665B is then washed, leaving only the HRP-detection antibody conjugate where it is bound to the target analyte. Thus, at this point, the amount of HRP present and bound in a recess 520 is proportional to the amount of target analyte bound to multiplexed volumetric bar chart chip 500, and hence, proportional to the amount of target analyte present in the sample. As will hereinafter be discussed in greater detail, when hydrogen peroxide from ultimate sample row 665A is thereafter introduced into penultimate sample row 665B (via an oblique slide of top plate 510 relative to bottom plate 515), the reaction between the hydrogen peroxide and the HRP will generate nitrogen gas in an amount proportional to the amount of HRP present (and hence, proportional to the amount of target analyte present). See FIG. 54H.

2. The “Competitive” ELISA Assay

When the “competitive” ELISA method is used for the assay, the principle is similar to that of the aforementioned “sandwich” ELISA method, however, with this type of assay the target analyte (i.e., the analyte being tested for) will bind to the analyte-specific antibody which is bound to a recess 520 of continuous penultimate sample row 665B, and an HRP-drug derivative conjugate will bind to the analyte-specific antibody which is bound to a recess 520 of continuous penultimate sample row 665B only where the analyte-specific antibody has not already bound the target analyte, as will hereinafter be discussed in greater detail.

First, the sample is introduced into inlet 555 (FIG. 54E) of multiplexed volumetric bar chart chip 500 so that the sample fills continuous penultimate sample row 665A. After a period of time, continuous penultimate sample row 665A is washed, leaving the target analyte(s) (where present) bound to the analyte-specific antibodies which are, in turn, bound to a recess 520 of continuous penultimate sample row 665B. At this point, the amount of the target analyte retained in sample row 665B is proportional to the amount of target analyte present in the sample.

A conjugate comprising horseradish peroxidate (HRP) bound to a drug derivative (which drug derivative is selected because it will bind the analyte-specific antibodies bound to recess 520) is prepared. The HRP-drug derivative conjugate is introduced into inlet 555 of multiplexed bar chart chip 500. The HRP-drug derivative conjugate will only bind the analyte-specific antibodies where the target analyte is absent (i.e., where the target analyte has not bound to the analyte-specific antibodies). Thus, at this point, the amount of HRP present and bound in recess(es) 520 of continuous penultimate sample row 665B is inversely proportional to the amount of target analyte present in the sample. As will hereinafter be discussed in greater detail, when hydrogen peroxide from continuous ultimate sample row 665A is thereafter introduced into continuous penultimate sample row 665B (via an oblique slide of top plate 510 relative to bottom plate 515), the reaction between the hydrogen peroxide and the HRP will generate nitrogen gas in an amount proportional to the amount of HRP present (and hence, inversely proportional to the amount of target analyte present). See FIG. 54I.

3. Control Preparation

Regardless of whether the “sandwich” ELISA method or the “competitive” ELISA method is utilized, continuous penultimate control row 675B (FIG. 54E) will contain a predetermined amount of HRP. More particularly, a solution containing HRP is prepared, with the concentration of HRP being selected so as to reflect the target “threshold” for detecting the target analyte, as will hereinafter be discussed in greater detail.

The HRP solution is introduced into inlet 575 of multiplexed volumetric bar chart chip 500, whereby to fill continuous penultimate control row 675B. As will also hereinafter be discussed in greater detail, when hydrogen peroxide from continuous ultimate control row 675A is thereafter introduced into penultimate control row 675B (via an oblique slide of top plate 510 relative to bottom plate 515), the reaction between the hydrogen peroxide and the HRP will generate nitrogen gas in an amount proportional to the amount of HRP present (i.e., proportional to the concentration of the HRP selected as the control).

4. Initiating the Assay

Irrespective of whether the “sandwich” ELISA method or the “competitive” ELISA method is used, the assay is completed in the same fashion. More particularly, after the sample has been loaded into continuous penultimate sample row 665B (FIG. 54E), and after HRP has been bound in continuous penultimate sample row 665B, and after a known concentration of HRP has been loaded into continuous penultimate control row 675B (i.e., via either of the foregoing methods), top plate 510 is slid obliquely relative to bottom plate 515, causing continuous sample rows 665, continuous control rows 670 and continuous ink row 675 to be disrupted and transformed into continuous bar channels 505 (i.e., 505A, 505B, 505C, etc.), such as shown in FIG. 54B. As this row-to-column transformation occurs, each recess 520 (containing the analyte-specific antibodies and any target analytes bound thereto and any HRP bound thereto) previously located in continuous penultimate sample row 665B or continuous penultimate control row 675B becomes incorporated as a section of a specific bar channel 505 (i.e., 505A, 505B, 505C, etc.). In addition, as this row-to-column transformation occurs, the hydrogen peroxide contained in continuous ultimate sample row 665A and continuous ultimate control row 675A is permitted to advance up each of the bar channels 505 and thereby mix with any HRP bound to the analyte-specific antibodies, the mixing of which causes a reaction which releases nitrogen gas. The nitrogen gas is produced in proportion to the quantity of HRP present in a given penultimate sample row 665B and a given penultimate control row 675B. As nitrogen gas is produced by the reaction between hydrogen peroxide and HRP in penultimate sample row 665B, the nitrogen gas passes through bar channels 660 and contacts ink residing in ink row 685, whereby to propel the ink into bar channels 600 (i.e., nitrogen gas passes up bar channel 505 from sample portion 670, whereby to propel ink from ink row 685 up bar channel 505 and into control portion 680).

Simultaneously, nitrogen gas is produced by the reaction between hydrogen peroxide and HRP located in penultimate control row 675B, causing nitrogen gas to pass through bar channels 600 and contact ink residing in ink row 685, whereby to propel the ink into bar channels 660 (i.e., nitrogen gas passes down bar channel 505 from control portion 680, whereby to propel ink from ink row 685 down bar channel 505 and into sample portion 670).

By virtue of this construction, the nitrogen gas produced by the control and the nitrogen gas produced by the sample are directed against one another and “compete” in order to move the ink residing in ink row 685. Put another way, the ink will move into bar channels 600 (i.e., up bar channels 505) if there is a greater amount of HRP in penultimate sample row 665B than there is HRP in penultimate control row 675B (i.e., the nitrogen gas generated by the sample “out competes” the nitrogen gas generated by the control).

Conversely, the ink will move into bar channels 660 (i.e., down bar channels 505) if there is a greater amount of HRP in penultimate control row 675B than there is HRP in penultimate sample row 665B (i.e., the nitrogen gas generated by the control “out competes” the nitrogen gas generated by the sample).

As discussed above, the amount of HRP located in penultimate control row 675B is equal to the concentration of the HRP introduced into penultimate control row 675B (i.e., via inlet 575). Since this concentration is known, the amount of nitrogen gas produced by the reaction of HRP in penultimate control row 675B with hydrogen peroxide is also known, thereby allowing one to set a predetermined “threshold” amount of nitrogen gas which will need to be generated by the reaction between HRP in penultimate sample row 665B with hydrogen peroxide, in order for the sample to produce enough nitrogen gas to “outcompete” the nitrogen gas produced by the sample and thereby propel ink out of ink row 685 and into bar channels 600 (i.e., up bar channels 505). Thus, by setting the amount of HRP located in penultimate control row 675B, one is able to set the threshold level of the target analyte which must be present in the sample in order to yield a “positive” test result.

Furthermore, inasmuch as the ink is propelled either up bar channels 505, or down bar channels 505, a distance along bar channels 505 which is proportional to the quantity of nitrogen gas produced in that bar channel, which is in turn proportional to the quantity of the target analytes which are bound to the analyte-specific antibodies (or HRP-drug derivative conjugate, which quantity is inversely proportional to the quantity of target analytes) disposed in the recesses associated with that column, the quantity of the analyte present in the sample can be determined by viewing the direction and distance that the ink travels within bar channels 505.

As a result of the foregoing, by disposing different analyte-specific antibodies in different ones of the recesses 520 (FIG. 54C) of continuous penultimate sample row 665A, multiplexed volumetric bar chart chip 500 can be used to simultaneously determine (i) whether an analyte is present in a sample at a concentration above a predetermined threshold, and (ii) the quantity of multiple analytes present in a sample, with the quantity of each analyte being indicated in a particular one of a plurality of bar channels 505. See, for example, FIGS. 54J-54M which show the test results for various samples.

FIG. 54N lists some exemplary analytes and exemplary threshold (i.e., “cutoff”) values which may be used when selecting a concentration of HRP to be used for the control.

Thus it will be appreciated that the amount of HRP present in penultimate sample row 665B corresponds to the amount of target analyte present in penultimate sample row 665B, and the amount of HRP present in penultimate control row 675B corresponds to the amount of HRP introduced into penultimate control row 675B by the user to act as the control for the “competitive” multiplexed volumetric bar chart chip 500. It will also be appreciated that the direction of movement of ink out of ink row 685 and into bar channels 600 is governed by the difference between the amount of nitrogen gas generated by the reaction of the HRP present in penultimate sample row 665B and the amount of nitrogen gas generated by the reaction of the HRP present in penultimate control row 675B. Put another way, the ink will move into bar channels 600 in a direction away from the reaction which produces a greater amount of nitrogen gas (and towards the reaction which produces a lesser amount of nitrogen gas).

Significantly, the “competitive” multiplexed volumetric bar chart chip 500 has a wide range and high precision.

More particularly, it has been found that very small differences in the amount of HRP present in penultimate sample row 665B and the amount of HRP present in penultimate control row 675B results in discernable movement of ink within ink row 685.

By way of example but not limitation, where the recesses of sample row 665B and the recesses of control row 675B contain equal volumes of sample and control, a difference between a concentration of 1 μM HRP (disposed in one of sample row 665B and control row 675B) and a concentration of 4.5 μM HRP (disposed in the other of sample row 665B and control row 675B) causes ink to move toward the 1 μM HRP side of the multiplexed volumetric bar chart chip.

By way of further example but not limitation, where the recesses of sample row 665B and the recesses of control row 675B contain equal volumes of sample and control, a difference between a concentration of 4 μM HRP (disposed in one of sample row 665B and control row 675B) and a concentration of 5 μM HRP (disposed in the other of sample row 665B and control row 675B) causes ink to move toward the 4 μM HRP side of the multiplexed volumetric bar chart chip.

In addition, it has also been found that very small differences in the quantity of HRP present in a first sample (i.e., the target analyte) vs the quantity of HRP present in a second sample (i.e., the target analyte) results in discernably different advancement of the ink into bar channels 600 when offset by the same control.

By way of example but not limitation, where the recesses of sample row 665B and the recesses of control row 675B contain equal volumes of sample and control, a difference between a concentration of 4 μM HRP in a first sample and 6 μM HRP in a second sample, using a control with a concentration of 5 μM HRP, causes a discernable difference in the distance that the ink advances along bar channels 600 (i.e., the ink moves along bar channels 600 toward control row 675B with the second sample and moves toward sample row 665B with the first sample, since the second sample contains a greater amount of HRP than the first sample).

It should be appreciated that this sensitivity can contribute to the ability of “competitive” multiplexed volumetric bar chart chip 500 to distinguish between target concentrations which are close to a threshold value and can reduce false-negative and false-positive results.

It should also be appreciated that the distance that the ink moves along bar channels 600 is a function of the amount of HRP present in the sample and control, and that the multiplexed volumetric bar chart chip of the present invention accommodates a wide range of sample and control concentrations. It has been found that the ink within ink row 685 moves a discernable distance along bar channels 600 when the concentration of HRP present in sample row 665B (which is a function of the concentration of the analyte present in sample row 665B) is quite high (e.g., >100 μM HRP), and that the ink within ink row 685 also moves a discernable distance along bar channels 600 when the concentration of HRP present in sample row 665B (which is a function of the concentration of the analyte present in sample row 665B) is quite low (e.g., in the sub-millimole and nanomole concentration range), provided that the sample is opposed by a similarly concentrated, but lesser concentrated, HRP control disposed in control row 675B.

By way of example but not limitation, where the recesses of sample row 665B and the recesses of control row 675B contain equal volumes of sample and control, the difference between a concentration of 500 μM HRP (disposed in one of sample row 665B and control row 675B) and a concentration of 750 μM HRP (disposed in the other of sample row 665B and control row 675B) causes ink to move toward the 500 μM HRP side of multiplexed volumetric bar chart chip 500.

By way of further example but not limitation, where the recesses of sample row 665B and the recesses of control row 675B contain equal volumes of sample and control, the difference between a concentration of 5 nM HRP (disposed in one of sample row 665B and control row 675B) and a concentration of 7.5 nM HRP (disposed in the other of sample row 665B and control row 675B) causes ink to move toward 5 nM HRP side of multiplexed volumetric bar chart chip.

The novel method and apparatus of the present invention provides instant and visual quantitation of target biomarkers or other molecular analytes and provides a visualized bar chart without the use of instruments, data processing or graphic plotting. Thus, since the novel method and apparatus of the present invention does not require the use of complex instruments, the novel method and apparatus of the present invention can be easily used as a point of care determination of the quantity of an analyte (and, preferably, the quantity of multiple analytes) present in a sample. More particularly, the novel method and apparatus of the present invention can be used as a point of care determination of the quantity of protein, nucleic acid, peptide, sugar, organic compounds, polymer, metal ions, and/or other molecular analytes, as well as the quantity of bacteria, cells, and/or particles.

### Multiplexed Volumetric Bar Chart Chip Utilizing Horizontal Slide and Serpentine Channels

In another embodiment of the present invention, and looking now at FIG. 54O, a novel multiplexed volumetric bar chart chip is provided which may be used in accordance with the present invention to determine the quantity of a target protein (or other types of biomarkers or other analytes) present in a sample.

More particularly, and still looking now at FIG. 54O, in this form of the invention, there is provided a novel multiplexed volumetric bar chart chip 700 which is configured to determine the quantity of multiple analytes which may be present in a sample, with the quantity of each analyte which is present in the sample being indicated in a particular one of a plurality of serpentine channels 705. Serpentine channels 705 can be advantageous inasmuch as they provide an increased channel length without increasing the overall size of multiplexed volumetric bar chart chip 700. By way of example but not limitation, 3, 6, 10, 20, 30, and 50-plexed, or more than 50-plexed, serpentine channels may be incorporated into multiplexed volumetric bar chart chip 700. In one preferred embodiment of the present invention, multiplexed volumetric bar chart chip 700 comprises three serpentine channels. It should be appreciated that, although serpentine channels 705 are shown as S-shaped channels, serpentine channels 705 may be straight or curved (e.g., circular, z-shaped) or formed in any other configuration which provides a series of channels having a length. As a result of this construction, the review of a particular serpentine channel 705 will indicate the quantity of a particular analyte which may be present in the sample and, significantly, the collective array of the plurality of serpentine channels 705 will simultaneously indicate the presence of, and quantities of, multiple analytes which may be present in the sample, whereby to provide multi-analyte quantity measurements and hence a more comprehensive diagnostic result.

For the sake of clarity, the multiplexed volumetric bar chart chip 700 will be discussed in the context of a three-plexed volumetric bar chart chip.

Looking now at FIGS. 54P, 54Q and 54R, multiplexed volumetric bar chart chip 700 comprises two plates, a transparent top plate 710 and a bottom plate 715 (which may or may not be transparent).

More particularly, top plate 710 (FIG. 54P) has a plurality of sample recesses 720 formed on its bottom surface, with sample recesses 720 being arranged along the upper portion of the bottom surface of top plate 710 so as to provide a plurality of inlets 725 and a plurality of outlets 730 for facilitating loading of a sample into a sample well, as will hereinafter be discussed in greater detail. Top plate 710 also has a plurality of reaction wells 735 formed on its bottom surface, with reaction wells 735 being arranged along the upper portion of the bottom surface of top plate 710. A plurality of inlets 740 and a plurality of outlets 745 are also formed in top plate 710, adjacent reaction wells 735, for permitting loading of a reactant into reaction wells 735 as will hereinafter be discussed in greater detail.

A plurality of ink recesses 750 are formed in the bottom surface of top plate 710 and extend horizontally across the bottom surface of top plate 710. An inlet 755 is connected to a far side ink recess 750, and an outlet 760 is formed adjacent to the opposite far side ink recess 750.

A plurality of connection recesses 765 are formed in the bottom surface of top plate 710, disposed intermediate reaction wells 735 and ink recesses 750.

In addition, a plurality of serpentine channels 705 are formed on the bottom surface of top plate 710, with each of the serpentine channels 705 extending between (although not in fluid communication with) ink recesses 750 and the bottom edge of top plate 710. Serpentine channels 705 comprise an inlet 770 disposed at the end of each serpentine channel 705 which is adjacent ink recesses 750 and an outlet 775 disposed at the opposite end of each serpentine channel 705 (i.e., adjacent the bottom edge of top plate 710).

In one preferred form of the invention, sample recesses 720, reaction wells 735, ink recesses 750, serpentine channels 705, inlets 725, 740, 755, 770 and outlets 730, 745, 760, 775 are all formed in the bottom surface of top plate 710 using a conventional etching process of the sort well known in the etching arts. Preferably, sample recesses 720, reaction wells 735, ink recesses 750, serpentine channels 705, inlets 725, 740, 755, 770 and outlets 730, 745, 760, 775 are etched in the bottom surface of a glass plate. Alternatively, sample recesses 720, reaction wells 735, ink recesses 750, serpentine channels 705, inlets 725, 740, 755, 770 and outlets 730, 745, 760, 775 may be formed in a silicon plate, a plastic plate, a ceramic plate, a quartz plate, a metal oxide plate or other appropriate substrate material.

Bottom plate 715 (FIG. 54Q) has a plurality of reactant recesses 780 formed on its top surface, with reactant recesses 780 being arranged such that, when top plate 710 is disposed over bottom plate 715 (i.e., when novel multiplexed volumetric bar chart chip 700 is assembled), reactant recesses 780 fluidically connect inlet 740 and outlet 745 to reaction wells 735 (FIG. 54R).

Bottom plate 715 also has a plurality of sample wells 785 (FIG. 54Q) formed on its top surface, with sample wells 785 being arranged along the upper portion of the top surface of bottom plate 715. Sample wells 785 are arranged such that, when top plate 710 is disposed over bottom plate 715 (i.e., when novel multiplexed volumetric bar chart chip 700 is assembled), sample recesses 720 connect inlet 725 and outlet 730 to sample wells 785 (FIG. 54R).

Bottom plate 715 also has a plurality of horizontally-extending connection recesses 790 (FIG. 54Q) formed on its top surface, with connection recesses 790 being arranged along the upper portion of the top surface of bottom plate 715. Connection recesses 790 are arranged such that, when top plate 710 is disposed over bottom plate 715 (i.e., when novel multiplexed volumetric bar chart chip 700 is assembled), connection recesses 790 fluidically link ink recesses 750 of top plate 710 together (FIG. 54R), whereby to form a continuous ink row and fluidly connect inlet 755 and outlet 760 as will hereinafter be discussed in greater detail.

A plurality of connection recesses 795 (FIG. 54Q) are formed in the top surface of bottom plate 715, disposed below sample wells 785. Connection recesses 795 are arranged such that, when top plate 710 is disposed over bottom plate 715 (i.e., when novel multiplexed volumetric bar chart chip 700 is assembled), connection recesses 795 are in fluid communication with ink recesses 750 (FIG. 54S), whereby to permit connection recesses 795 to be filled with liquid ink when ink recesses 750 are filled with liquid ink, as will hereinafter be discussed in greater detail.

In one preferred form of the invention, reactant recesses 780, sample wells 785 and connection recesses 790, 795 are all formed in the top surface of bottom plate 715 using a conventional etching process of the sort well known in the etching arts. Preferably, reactant recesses 780, sample wells 785 and connection recesses 790, 795 are etched in the top surface of a glass plate. Alternatively, reactant recesses 780, sample wells 785 and connection recesses 790, 795 may be formed in a silicon plate, a plastic plate, a ceramic plate, a quartz plate, a metal oxide plate or other appropriate substrate material.

1. Assembly of Multiplexed Volumetric Bar Chart Chip 700

Looking next at FIGS. 54R, 54S and 54T, top plate 710 is assembled on top of bottom plate 715 so that sample recesses 720 in top plate 710 will cooperate with sample wells 785 in bottom plate 715 so as to permit loading of a sample to be assayed into a given sample well 785 via a given inlet 725. Reactant recesses 780 in bottom plate 715 will cooperate with reaction wells 735, inlet 740 and outlet 745 in top plate 710 so as to permit loading of a reactant into a given reaction well 735 via a given inlet 740. Connection recesses 790 will cooperate with ink recesses 750 so as to form a continuous ink row fluidically connecting inlet 755 to outlet 760, whereby to permit loading of a liquid ink (e.g., red ink) into ink recesses 750. To assist in visualizing the assembly of top plate 710 and bottom plate 715, FIG. 54S is a schematic view of top plate 710 and bottom plate 715, with the structures of top plate 710 outlined in solid line and the structures of bottom plate 715 outlined in dashed line.

It will be appreciated that, due to the dispositions of recesses 720, 750, 765 and reaction wells 735 in top plate 710, and recesses 780, 790, 795 and sample wells 785 in bottom plate 715, a horizontal slide (to the left) of top plate 710 relative to bottom plate 715 disrupts the aforementioned configuration of FIG. 54S to provide the configuration of FIG. 54T. More particularly, when top plate 710 is slid horizontally (to the left) relative to bottom plate 715, (i) reaction wells 735 move horizontally relative to sample wells 785, thereby combining reaction wells 735 with sample wells 785, whereby to mix the contents of reaction wells 735 with the contents of sample wells 785, (ii) ink recesses 750 and serpentine channels 705 (and inlet 770 of serpentine channels 705) are shifted horizontally, whereby to align with, and fluidically connect, a given inlet 770 of serpentine channels 705 with a given connection recess 795 of bottom plate 715, and (iii) connection recesses 765 are shifted horizontally, whereby to align with, and fluidically connect, a given connection recess 795 of bottom plate 715 with a given sample well 785. To assist in visualizing the movement of top plate 710 relative to bottom plate 715, FIG. 54T is a schematic view of top plate 710 and bottom plate 715, with the structures of top plate 710 outlined in solid line and the structures of bottom plate 715 outlined in dashed line, and showing multiplexed volumetric bar chart chip 700 after top plate 710 has been slid horizontally relative to bottom plate 715.

By virtue of this construction, it will be appreciated that when top plate 710 is slid horizontally (to the left) relative to bottom plate 715, the sample and the reactant are mixed together, whereby to generate a gas, which gas exits the combined sample well 785/reaction well 735 through connection recess 765, forces liquid ink out of connection recess 795, through inlet 770 and hence, forces liquid ink into serpentine channels 705. It will further be appreciated that, by virtue of the foregoing construction, the distance that ink travels in a given serpentine channel 705 is proportional to the amount of gas generated by the reaction between the sample and the reactant. Thus, by using an assay which generates an amount of gas that is proportional to the amount of a given analyte present in a sample, viewing of a particular serpentine channel 705 indicates the quantity of a given analyte present in a sample, as will hereinafter be discussed in greater detail.

2. Determining the Quantity of Multiple Analytes Present in a Sample Using Multiplexed Volumetric Bar Chart Chip 700

In view of the foregoing construction, multiplexed volumetric bar chart chip 700 can be used to determine the quantity of multiple analytes present in a sample, with the quantity of each specific analyte being indicated in a particular one of the plurality of serpentine channels 705.

More particularly, and referring now to FIG. 54R, and as will hereinafter be discussed in further detail below, during manufacture of multiplexed volumetric bar chart chip 700, an analyte-specific antibody is bonded in sample wells 785 of bottom plate 715 of multiplexed volumetric bar chart chip 700. In a preferred form of the present invention, a different analyte-specific antibody is bonded in each sample well 785.

Prior to use, hydrogen peroxide (H₂O₂) is introduced into inlet 740 of multiplexed volumetric bar chart chip 700, whereby to fill reaction wells 735 with hydrogen peroxide. Red ink (or some other colored material which is readily discernible through top plate 710 and against bottom plate 715) is introduced into inlet 755 of multiplexed volumetric bar chart chip 700, whereby to fill ink recesses 750 (and also connection recesses 795, which are in fluid communication with ink recesses 750) of multiplexed volumetric bar chart chip 700 with red ink.

It should be appreciated that the foregoing construction may be utilized with different types of assays. By way of example but not limitation, the assays utilizing the “sandwich” ELISA method and the “competitive” ELISA method discussed above may also be used with multiplexed volumetric bar chart chip 700. By way of further example but not limitation, a variation of the “sandwich” ELISA method discussed above may be used for the assay, as will hereinafter be discussed in further detail.

3. “Sandwich” ELISA Assay Utilizing Platinum Nanoparticles

When the “sandwich” ELISA method utilizing platinum nanoparticles is used for the assay, the target analyte (i.e., the analyte being tested for) will bind the analyte-specific antibody which is bound to a sample well 785 of multiplexed volumetric bar chart chip 700, as will hereinafter be discussed in greater detail.

First, the sample is introduced into inlet 725 (FIG. 54R) of multiplexed volumetric bar chart chip 700, so that the sample fills a given sample well 785. After a period of time, the sample wells 785 are washed, leaving the target analyte(s) (where present) bound to the analyte-specific antibodies which are, in turn, bound to sample well 785. At this point, the amount of the target analyte retained in a given sample well 785 is proportional to the amount of the target analyte present in the sample.

A conjugate comprising platinum nanoparticles bound to a detection antibody (which detection antibody is selected because it will bind the target analyte) is then prepared. The platinum nanoparticle-detection antibody conjugate is introduced into inlet 725 of multiplexed bar chart chip 700, so that the platinum nanoparticle-detection antibody conjugate fills a given sample well 785. Where the target analyte is present (i.e., where the target analyte is bound to an analyte-specific antibody bound to sample well 785), the platinum nanoparticle-detection antibody conjugate will bind the target analyte. Sample wells 785 are then washed, leaving only those platinum nanoparticle-detection antibody conjugates which are bound to the target analyte. Thus, at this point, the amount of platinum nanoparticles present and bound in a given sample well 785 is proportional to the amount of target analyte bound to multiplexed volumetric bar chart chip 700, and hence, proportional to the amount of target analyte present in the sample. As will hereinafter be discussed in greater detail, when hydrogen peroxide from reaction well 735 is thereafter introduced into sample well 785 (via a horizontal slide of top plate 710 relative to bottom plate 715), the reaction between the hydrogen peroxide and the platinum nanoparticles will generate oxygen gas in an amount proportional to the amount of platinum nanoparticles present (and hence, proportional to the amount of target analyte present). See FIG. 54U.

4. Initiating the Assay

Irrespective of which assay is used, the assay is completed in the same fashion. After the sample has been loaded into sample wells 785, and after platinum nanoparticles have been bound in sample wells 785 as discussed above, top plate 710 is slid horizontally (FIG. 54O) relative to bottom plate 715, causing reaction wells 735 to communicate with sample wells 785, and aligning sample wells 785 with connection recesses 765, which is in turn aligned with, and in fluid communication with, connection recess 795 (which connection recess 795 contains red ink), which is, in turn, aligned with, and in fluid communication with, inlet 760 of serpentine channels 705. See FIGS. 54S and 54T. Thus, the hydrogen peroxide is introduced to the analyte. As the hydrogen peroxide mixes with the analyte, the oxygen gas produced passes through connection recess 765, forces liquid ink out of connection recess 795, through inlet 770 and hence, forces liquid ink into serpentine channels 705. Thus, by virtue of the foregoing construction, the distance that ink travels in a given serpentine channel 705 is proportional to the amount of oxygen gas generated by the reaction between the sample (i.e., the analyte) and the reactant (i.e., the hydrogen peroxide). Thus, a review of a particular serpentine channel 705 indicates the quantity of a given analyte present in a sample.

As a result of the foregoing, by disposing different analyte-specific antibodies in different ones of the sample wells 785, multiplexed volumetric bar chart chip 700 can be used to determine the quantity of multiple analytes present in a sample, with the quantity of each analyte being indicated in a particular one of a plurality of serpentine channels 705. See, for example, FIGS. 54U-54W which show the test results for various samples.

By way of example but not limitation, some exemplary analytes may include, but are not limited to, interleukin-1 receptor antagonist (IL-1RA), soluble tumor necrosis factor receptor II (sTNF-RII), and soluble interleukin 1 receptor, type II (IL-1SR2).

The novel method and apparatus of the present invention provides instant and visual quantitation of target biomarkers or other molecular analytes and provides a visualized bar chart without the use of instruments, data processing or graphic plotting. Thus, since the novel method and apparatus of the present invention does not require the use of complex instruments, the novel method and apparatus of the present invention can be easily used as a point of care determination of the quantity of an analyte (and, preferably, the quantity of multiple analytes) present in a sample. More particularly, the novel method and apparatus of the present invention can be used as a point of care determination of the quantity of protein, nucleic acid, peptide, sugar, organic compounds, polymer, metal ions, and/or other molecular analytes, as well as the quantity of bacteria, cells, and/or particles.

### Exemplary Assay—HCC Assessment

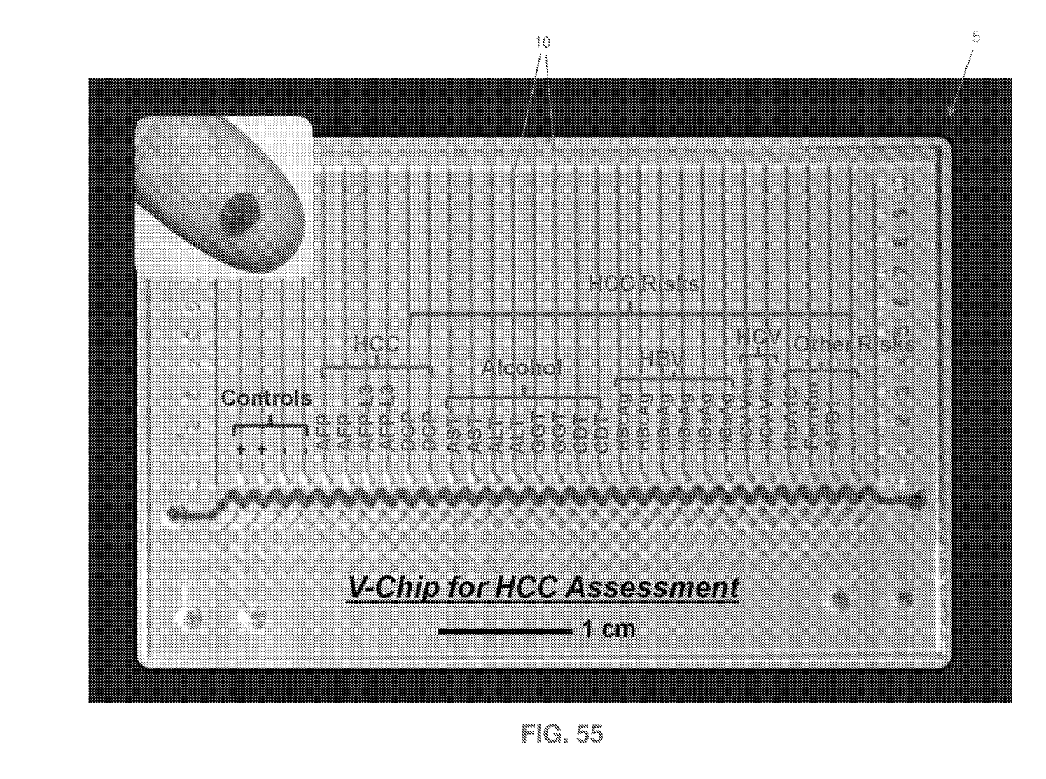

In one preferred form of the invention, multiplexed volumetric bar chart chip 5 may be utilized for a hepatocellular carcinoma risk assessment assay. More particularly, and looking now at FIG. 55, one or more biomarkers (e.g., biomarkers which are linked to hepatocellular carcinoma) may be assayed by preparing multiplexed volumetric bar chart chip 5 with biomarker-specific antibodies bound to recesses 30 of row 75B. By way of example but not limitation, the biomarkers may comprise one or more from the group consisting of AFP, AFP-L3, DCP, AST, ALT, GGT, CDT, HBcAg, HBeAg, HBsAg, HCV Virus, HbA1C, Ferritin and AFB1.

### Exemplary Assay—Breast Cancer Diagnosis

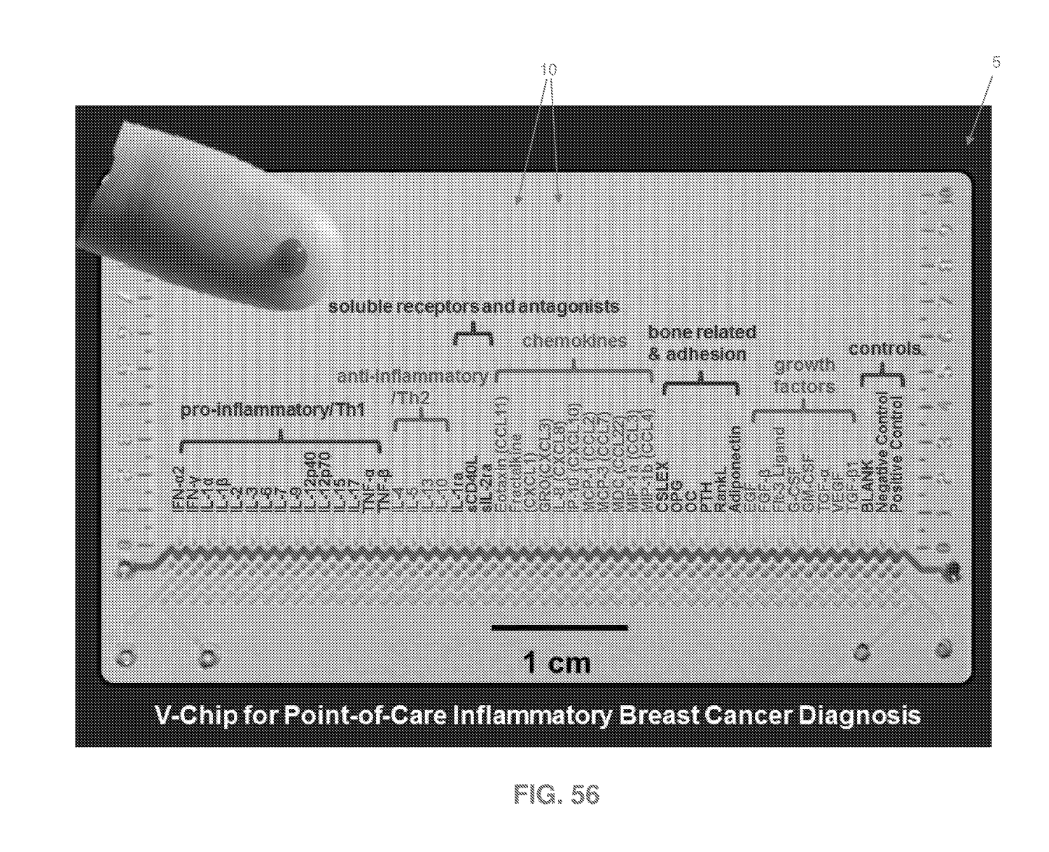

In another preferred form of the invention, multiplexed volumetric bar chart chip 5 may be utilized for a breast cancer risk/diagnosis assay. More particularly, and looking now at FIG. 56, one or more biomarkers (e.g., biomarkers which are linked to breast cancer/risk of breast cancer) may be assayed by preparing multiplexed volumetric bar chart chip 5 with biomarker-specific antibodies bound to recesses 30 of row 75B. By way of example but not limitation, the biomarkers may comprise one or more from the group consisting of IFN-α2, IFN-γ, IL-1α, IL-1β, IL-2, IL-3, IL-6, IL-7, IL-9, IL-12p40, IL-12p70, IL-15, IL-17, TNF-α, TNF-β, IL-4, IL-5, IL-13, IL-10, IL-1ra, sCD40L, sIL-2ra, Eotaxin (CCL11), Fractalkine, (CXCL1), GRO (CXCL3), IL-8 (CXCL8), IP-10 (CXCL10), MCP-1 (CCL2), MCP-3 (CCL7), MDC (CCL22), MIP-1a (CCL3), MIP-1b (CCL4), CSLEX, OPG, OC, PTH, RankL, Adiponectin, EGF, FGF-β, Flt-3 Ligand, G-CSF, GM-CSF, TGF-α, VEGF and TGF-β1, as well as controls.

### Exemplary Assay—Sepsis Assessment

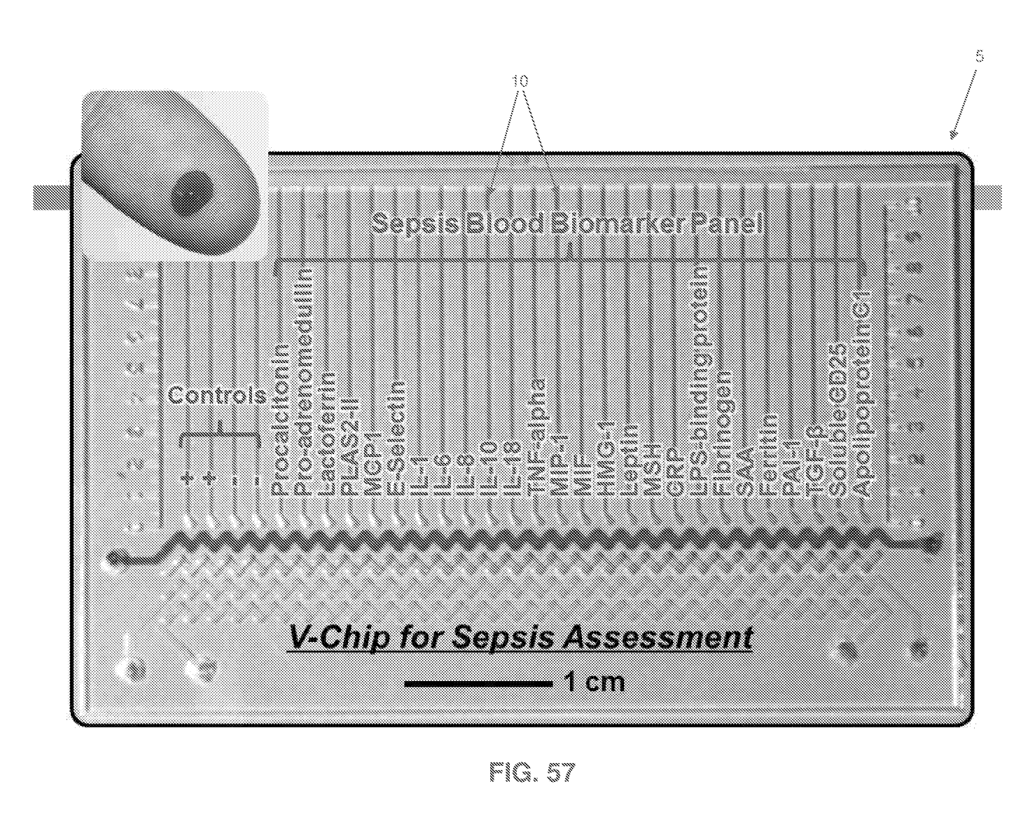

In another preferred form of the invention, multiplexed volumetric bar chart chip 5 may be utilized for a sepsis assessment assay. More particularly, and looking now at FIG. 57, one or more biomarkers (e.g., biomarkers which are linked to sepsis) may be assayed by preparing multiplexed volumetric bar chart chip 5 with biomarker-specific antibodies bound to recesses 30 of row 75B. By way of example but not limitation, the biomarkers may comprise one or more from the group consisting of Procalcitonin, Pro-adrenomedullin, Lactoferrin, PLAS2-II, MCP1, E-Selectin, IL-1, IL-6, IL-8, IL-10, IL-18, TNF-alpha, MIP-1, MIF, HMG-1, Leptin, MSH, CRP, LPS-binding protein, Fibrinogen, SAA, Ferritin, PAI-1, TGF-β, Soluble CD25 and Apolipoprotein C1.

### Exemplary Assay—Drug Abuse Assessment

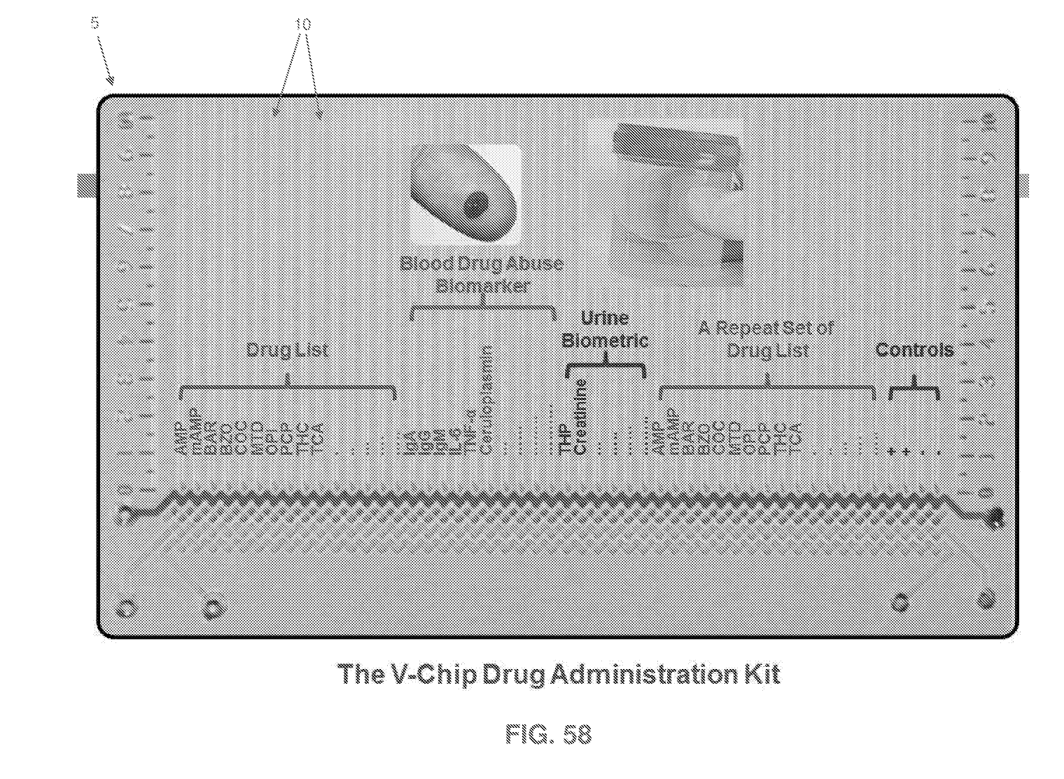

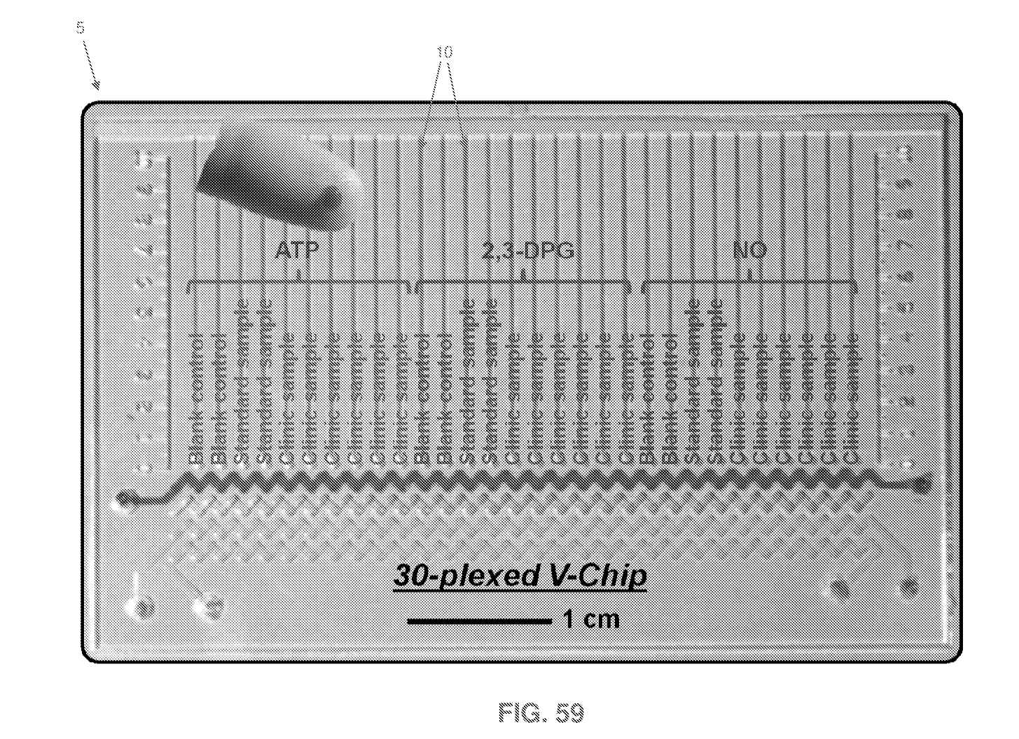

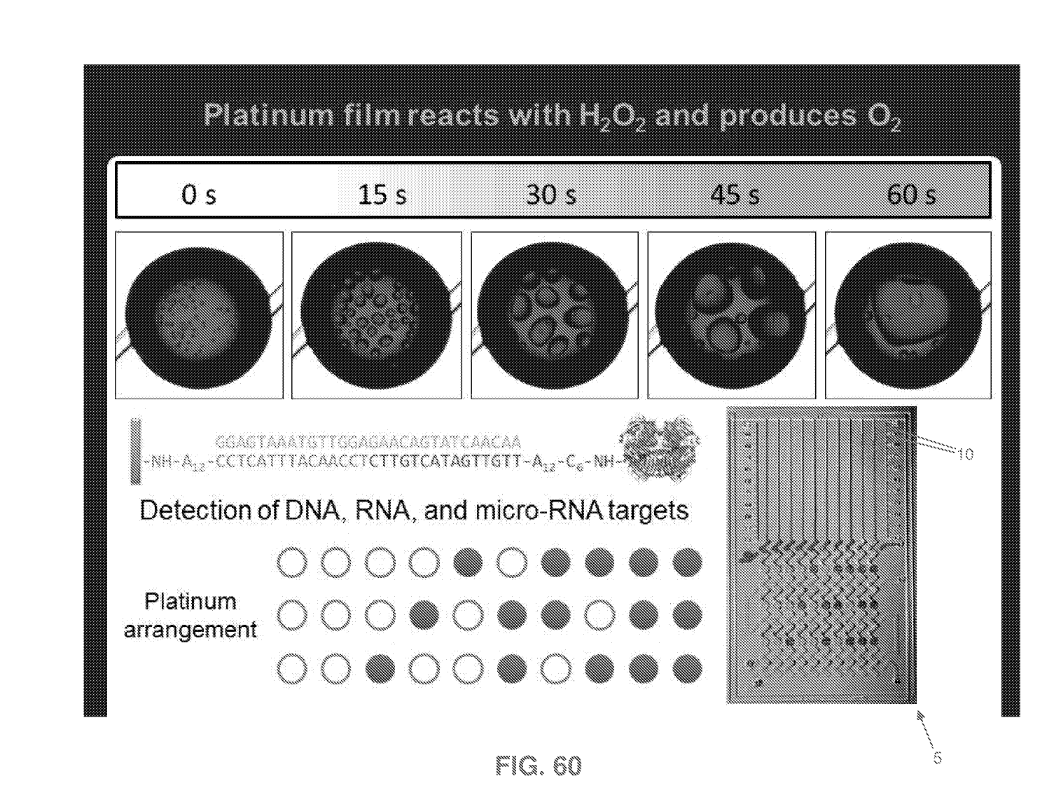

In another preferred form of the invention, multiplexed volumetric bar chart chip 5 may be utilized for a drug abuse assessment assay. More particularly, and looking now at FIG. 58, one or more biomarkers (e.g., biomarkers which are linked to drug abuse) may be assayed by preparing multiplexed volumetric bar chart chip 5 with biomarker-specific antibodies bound to recesses 30 of row 75B. By way of example but not limitation, the biomarkers may comprise one or more from the group consisting of AMP, mAMP, BAR, BZO, COC, MTD, OPI, PCP, THC, TCA, IgA, IgG, IgM, IL-6, TNF-α, Ceruloplasmin, THP and Creatinine.

### Exemplary Assay—30-Plexed V-Chip

In another preferred form of the invention, multiplexed volumetric bar chart chip 5 may be utilized for an assay of several important physiological biomarkers. More particularly, and looking now at FIG. 59, one or more biomarkers may be assayed by preparing multiplexed volumetric bar chart chip 5 with biomarker-specific antibodies bound to recesses 30 of row 75B. By way of example but not limitation, the biomarkers may comprise one or more from the group consisting of ATP, 2,3-DPG and NO.

### Exemplary Assay—Detection of DNA, RNA, and/or Micro-RNA

In another preferred form of the invention, multiplexed volumetric bar chart chip 5 may be utilized for an assay of DNA, RNA, and/or micro-RNA targets. More particularly, and looking now at FIG. 59, one or more DNA, RNA, and/or micro-RNA targets may be assayed by preparing multiplexed volumetric bar chart chip 5 with DNA, RNA, and/or micro-RNA-specific antibodies bound to recesses 30 of row 75B. If desired, a platinum film may be utilized to facilitate the assay.

### Additional Assays

In another preferred form of the invention, multiplexed volumetric bar chart chip 5 may be utilized for an assay of specific biomarkers/materials. By way of example but not limitation, the biomarkers may comprise one or more from the group consisting of IL-1RA, sTNFRII, IL-1SR2, ATP, 2,3-DPG, hemoglobin, NO and food allergens (e.g., peanut, pine nuts, etc.).

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for determining the quantity of a target analyte present in a sample, the apparatus comprising:
a first plate comprising a plurality of recesses arranged to form a plurality of rows extending parallel to one another, and a plurality of channels extending perpendicularly to the plurality of rows of the first plate; and
a second plate comprising a plurality of recesses arranged to form a plurality of rows extending parallel to one another, and a plurality of channels extending perpendicularly to the plurality of rows of the second plate;
wherein the first plate and the second plate are assembled together so that the first plate is positioned against the second plate and the recesses of the first plate communicate with the recesses of the second plate so as to form a plurality of sample rows, a plurality of control rows, and an ink row disposed between the plurality of sample rows and the plurality of control rows, with the plurality of channels of the first plate being disposed between the plurality of control rows and the ink row, and the plurality of channels of the second plate being disposed between the plurality of sample rows and the ink row; and
wherein at least one of the first plate and the second plate is configured to slide relative to the other of the first plate and the second plate in order to form a plurality of sample columns, a plurality of control columns and a plurality of ink columns, with each of the plurality of channels in the second plate being in communication with each of the plurality of sample columns and ink columns and with each of the plurality of channels in the first plate being in communication with each of the plurality of control columns and ink columns.

2. Apparatus according to claim 1 wherein the first plate is transparent.

3. Apparatus according to claim 1 wherein at least one of the plurality of rows formed in the first plate comprises an inlet and an outlet.

4. Apparatus according to claim 1 wherein the recesses in the first plate and the recesses in the second plate extend at a 45 degree angle relative to the axis of a row.

5. Apparatus according to claim 1 further comprising a analyte-specific antibody bound in at least one recess forming one of the plurality of rows of the second plate.

6. Apparatus according to claim 5 further comprising a sample positioned in the at least one recess containing the analyte-specific antibody.

7. Apparatus according to claim 6 further comprising a reactant positioned in the at least one recess containing the analyte-specific antibody and the sample.

8. Apparatus according to claim 7 wherein the reactant comprises catalase.

9. Apparatus according to claim 5 further comprising a plurality of analyte-specific antibodies each bound in a separate recess forming one of the plurality of rows of the second plate.

10. Apparatus according to claim 9 further comprising a sample positioned in each recess containing an analyte-specific antibody.

11. Apparatus according to claim 6 further comprising a reagent positioned in a recess in a row adjacent to the row containing the analyte-specific antibody.

12. Apparatus according to claim 11 wherein the reagent comprises hydrogen peroxide.

13. Apparatus according to claim 1 further comprising ink positioned in a recess in the ink row.

14. Apparatus according to claim 1 further comprising a bar code reader for detecting the disposition of ink contained in the plurality of channels in the first plate and channels in the second plate.

15. Apparatus according to claim 1 further comprising a known quantity of a reactant disposed in at least one recess forming one of the plurality of control rows.

16. Apparatus according to claim 15 further comprising a reagent disposed in at least one recess forming one of the plurality of control rows.

17. Apparatus according to claim 16 wherein the reactant of known quantity comprises catalase and the reagent comprises hydrogen peroxide.

18. A method for determining the quantity of a target analyte present in a sample, the method comprising:
providing apparatus comprising:
a first plate comprising a plurality of recesses arranged to form a plurality of rows extending parallel to one another, and a plurality of channels extending perpendicularly to the plurality of rows of the first plate; and
a second plate comprising a plurality of recesses arranged to form a plurality of rows extending parallel to one another, and a plurality of channels extending perpendicularly to the plurality of rows of the second plate;
wherein the first plate and the second plate are assembled together so that the first plate is positioned against the second plate and the recesses of the first plate communicate with the recesses of the second plate so as to form a plurality of sample rows, a plurality of control rows, and an ink row disposed between the plurality of sample rows and the plurality of control rows, with the plurality of channels of the first plate being disposed between the plurality of control rows and the ink row, and the plurality of channels of the second plate being disposed between the plurality of sample rows and the ink row; and
wherein at least one of the first plate and the second plate is configured to slide relative to the other of the first plate and the second plate in order to form a plurality of sample columns, a plurality of control columns and a plurality of ink columns, with each of the plurality of channels in the second plate being in communication with each of the plurality of sample columns and ink columns and with each of the plurality of channels in the first plate being in communication with each of the plurality of control columns and ink columns;
binding an analyte-specific antibody in at least one recess forming one of the plurality of sample rows of the second plate;
positioning a reagent in a recess adjacent to the sample row containing the analyte-specific antibody, positioning ink in a recess in the ink row, positioning a known concentration of a reactant in a control row, and positioning a reagent in a recess adjacent to the control row containing the known concentration of a reactant;
positioning a sample in the at least one recess containing the analyte-specific antibody;
positioning an analyte-specific antibody comprising a reactant in the at least one recess containing the analyte-specific antibody and the sample;
sliding one of the first plate and the second plate relative to the other of the first plate and the second plate so as to form the plurality of sample columns and control columns, with each sample column being in communication with one of the plurality of channels in the second plate and with each control column being in communication with one of the plurality of channels in the first plate; and
determining the quantity of the target analyte present in the sample by detecting the disposition of the ink contained in the plurality of channels in the second plate and the plurality of channels in the first plate.

19. A method according to claim 18 wherein the first plate is transparent.

20. A method according to claim 18 wherein at least one of the plurality of rows formed in the first plate comprises an inlet and an outlet.

21. A method according to claim 18 wherein the recesses in the first plate and the recesses in the second plate extend at a 45 degree angle relative to the axis of a row.

22. A method according to claim 18 wherein the reagent comprises hydrogen peroxide.

23. A method according to claim 18 wherein the reactant comprises catalase.

24. A method according to claim 18 further comprising a plurality of analyte-specific antibodies each bound in a separate recess forming one of the plurality of rows of the first plate.

25. A method according to claim 18 further comprising using a bar code reader to detect the longitudinal position of ink contained in the plurality of channels in the second plate and the plurality of channels in the first plate.

26. Apparatus for determining the quantity of a target analyte present in a sample, the apparatus comprising:
a control recess, a sample recess, an ink recess disposed between the control recess and the sample recess, and a channel fluidically connecting the control recess, the ink recess and the sample recess;
a known concentration of a reactant being disposed in the control recess, an analyte-specific antibody being bound in the sample recess, and ink being disposed in the ink recess;
means for introducing a sample into the sample recess;
means for introducing an analyte-specific antibody comprising a reactant into the sample recess; and
means for introducing a reagent into the sample recess for reacting with the reactant in the sample recess to produce a gas acting on the ink in the ink recess, and means for introducing a reagent into the control recess for reacting with reactant in the control recess to produce a gas also acting on the ink in the ink recess, whereby to enable determination of the quantity of the target analyte present in the sample by detecting the disposition of the ink in the channel.

27. A method for determining the quantity of a target analyte present in a sample, the method comprising:
providing apparatus comprising:
a control recess, a sample recess, an ink recess disposed between the control recess and the sample recess, and a channel fluidically connecting the control recess, the ink recess and the sample recess;
a known concentration of a reactant being disposed in the control recess, an analyte-specific antibody being bound in the sample recess, and ink being disposed in the ink recess;
introducing a sample into the sample recess;
introducing an analyte-specific antibody comprising a reactant into the sample recess; and
introducing a reagent into the sample recess for reacting with the reactant in the sample recess to produce a gas acting on the ink in the ink recess, and means for introducing a reagent into the control recess for reacting with reactant in the control recess to produce a gas also acting on the ink in the ink recess;
determining the quantity of the target analyte present in the sample by detecting the disposition of the ink in the channel.

28. Apparatus for determining the quantity of a target analyte present in a sample, the apparatus comprising:
a first plate comprising a first surface having a first recess containing an analyte-specific antibody and a second recess for containing ink, the first recess being spaced from the second recess;
a second plate comprising a second surface having a third recess for containing a reagent, a fourth elongated recess and a fifth recess, the fifth recess being disposed between, and spaced from, the third recess and the fourth elongated recess;
wherein the first plate and the second plate are assembled together so that the first surface of the first plate faces the second surface of the second plate;
wherein the first plate and the second plate are reconfigurable between (i) a first state in which the first recess is fluidically isolated from the third recess and the fifth recess and the second recess is fluidically isolated from the fourth elongated recess and the fifth recess, and (ii) a second state in which the first recess is fluidically connected to the third recess and the fifth recess and the second recess is fluidically connected to the fourth elongated recess and the fifth recess.

29. Apparatus according to claim 28 further comprising an inlet fluidically connected to the first recess and an outlet fluidically connected to the first recess.

30. Apparatus according to claim 29 further comprising a supply of a sample potentially containing the target analyte connected to the inlet.

31. Apparatus according to claim 30 further comprising a supply of an analyte-specific antibody connected to the inlet, wherein the supply of analyte-specific antibody comprises a reactant for reacting with a reagent disposed in the third recess when the first plate and the second plate are in the second state.

32. Apparatus according to claim 31 wherein the reagent comprises hydrogen peroxide and the reactant comprises catalase.

33. Apparatus according to claim 28 further comprising a second inlet fluidically connected to the second recess and a second outlet fluidically connected to the second recess.

34. Apparatus according to claim 33 further comprising a supply of ink connected to the second inlet for filling the second recess with ink.

35. Apparatus according to claim 28 further comprising a third inlet fluidically connected to the third recess and a third outlet fluidically connected to the third recess.

36. Apparatus according to claim 35 further comprising a supply of the reagent connected to the inlet.

37. Apparatus according to claim 28 further comprising a fourth outlet fluidically connected to the fourth elongated recess.

38. Apparatus according to claim 28 wherein the fourth elongated recess comprises a serpentine configuration.

39. Apparatus according to claim 28 wherein reconfiguring the first plate and second plate from their first state to their second state comprises moving one of the first plate and the second plate laterally relative to the other of the first plate and the second plate.

40. A method for determining the quantity of a target analyte present in a sample, the method comprising:
providing apparatus comprising:
a first plate comprising a first surface having a first recess containing an analyte-specific antibody and a second recess for containing ink, the first recess being spaced from the second recess;
a second plate comprising a second surface having a third recess for containing a reagent, a fourth elongated recess and a fifth recess, the fifth recess being disposed between, and spaced from, the third recess and the fourth elongated recess;
wherein the first plate and the second plate are assembled together so that the first surface of the first plate faces the second surface of the second plate;
wherein the first plate and the second plate are reconfigurable between (i) a first state in which the first recess is fluidically isolated from the third recess and the fifth recess and the second recess is fluidically isolated from the fourth elongated recess and the fifth recess, and (ii) a second state in which the first recess is fluidically connected to the third recess and the fifth recess and the second recess is fluidically connected to the fourth elongated recess and the fifth recess;
positioning the first plate and the second plate in their first state;
positioning a reagent in the third recess and positioning ink in the second recess;
positioning a sample in the first recess;
positioning an analyte-specific antibody comprising a reactant in the first recess so that the analyte-specific antibody comprising the reactant binds to any analyte present in the sample;
positioning the first plate and the second plate in their second state; and
determining the quantity of the analyte present in the sample by detecting the disposition of the ink in the fourth elongated recess.

41. A method according to claim 40 wherein the reagent comprises hydrogen peroxide and the reactant comprises catalase.

42. A method according to claim 40 wherein the fourth elongated recess comprises a serpentine configuration.

43. A method according to claim 40 wherein reconfiguring the first plate and second plate from their first state to their second state comprises moving one of the first plate and the second plate laterally relative to the other of the first plate and the second plate.
